(19) United States
(12) Patent Application Publication
(10) Pub. No.: US 2022/0192258 A1
(43) Pub. Date: Jun. 23, 2022

(54) SMOKING SUBSTITUTE APPARATUS

(71) Applicant: Nerudia Limited, Liverpool (GB)

(72) Inventors: Benjamin Illidge, Liverpool (GB); Benjamin Astbury, Liverpool (GB); Nikhil Aggarwal, Liverpool (GB); Peter Lomas, Liverpool (GB); Phillip Taylor, Liverpool (GB); Andrew Duckworth, Liverpool (GB); Matthew Pilkington, Liverpool (GB)

(21) Appl. No.: 17/693,992

(22) Filed: Mar. 14, 2022

Related U.S. Application Data
(63) Continuation of application No. PCT/EP20/76285, filed on Sep. 21, 2020.

(30) Foreign Application Priority Data
Sep. 20, 2019 (EP) 19198556.3
Sep. 20, 2019 (EP) 19198578.7
Sep. 20, 2019 (EP) 19198586.0
Sep. 20, 2019 (EP) 19198587.8
Sep. 20, 2019 (EP) 19198589.4
Sep. 20, 2019 (EP) 19198598.5
Sep. 20, 2019 (EP) 19198606.6
Sep. 20, 2019 (EP) 19198632.2
Sep. 20, 2019 (EP) 19198731.2

(51) Int. Cl. A24F 40/10; A24F 40/44; A24F 40/465; A24F 40/485

(52) U.S. Cl. CPC A24F 40/10; A24F 40/485; A24F 40/465; A24F 40/44

(57) ABSTRACT

A smoking substitute apparatus comprising an air inlet, a first passage leading from the air inlet to a first outlet, and an aerosol generator arranged in fluid communication with the first passage, the aerosol generator being operable to generate an aerosol from an aerosol precursor, to flow in use along the first passage downstream of the aerosol generator for inhalation by a user drawing on the outlet. The smoking substitute apparatus further comprises a second passage leading from the air inlet to a second outlet, separate from the first outlet, wherein the second passage bypasses the first passage downstream of the aerosol generator.

### CROSS-REFERENCE TO RELATED APPLICATIONS/INCORPORATION BY REFERENCE STATEMENT

This application is a non-provisional application claiming benefit to the international application no. PCT/EP2020/076285 filed on Sep. 21, 2020, which claims priority to EP 19198587.8 filed on Sep. 20, 2019, EP 19198586.0 filed on Sep. 20, 2019, EP 19198556.3 filed on Sep. 20, 2019, EP 19198731.2 filed on Sep. 20, 2019, EP 19198632.2 filed on Sep. 20, 2019, EP 19198589.4 filed on Sep. 20, 2019, EP 19198606.6 filed on Sep. 20, 2019, EP 19198598.5 filed on Sep. 20, 2019, and EP 19198578.7 filed on Sep. 20, 2019. The entire contents of each of the above-referenced applications are hereby incorporated herein by reference in their entirety.

### FIELD OF THE DISCLOSURE

The present disclosure relates to a smoking substitute apparatus and, in particular, a smoking substitute apparatus that is able to deliver nicotine to a user in an effective manner.

### BACKGROUND

The smoking of tobacco is generally considered to expose a smoker to potentially harmful substances. It is thought that a significant amount of the potentially harmful substances are generated through the burning and/or combustion of the tobacco and the constituents of the burnt tobacco in the tobacco smoke itself.

Low temperature combustion of organic material such as tobacco is known to produce tar and other potentially harmful by-products. There have been proposed various smoking substitute systems in which the conventional smoking of tobacco is avoided.

Such smoking substitute systems can form part of nicotine replacement therapies aimed at people who wish to stop smoking and overcome a dependence on nicotine.

Known smoking substitute systems include electronic systems that permit a user to simulate the act of smoking by producing an aerosol (also referred to as a “vapor”) that is drawn into the lungs through the mouth (inhaled) and then exhaled. The inhaled aerosol typically bears nicotine and/or a flavorant without, or with fewer of, the health risks associated with conventional smoking.

In general, smoking substitute systems are intended to provide a substitute for the rituals of smoking, whilst providing the user with a similar, or improved, experience and satisfaction to those experienced with conventional smoking and with combustible tobacco products.

The popularity and use of smoking substitute systems has grown rapidly in the past few years. Although originally marketed as an aid to assist habitual smokers wishing to quit tobacco smoking, consumers are increasingly viewing smoking substitute systems as desirable lifestyle accessories. There are a number of different categories of smoking substitute systems, each utilizing a different smoking substitute approach. Some smoking substitute systems are designed to resemble a conventional cigarette and are cylindrical in form with a mouthpiece at one end. Other smoking substitute devices do not generally resemble a cigarette (for example, the smoking substitute device may have a generally box-like form, in whole or in part).

One approach is the so-called “vaping” approach, in which a vaporizable liquid, or an aerosol former, sometimes typically referred to herein as “e-liquid”, is heated by a heating device (sometimes referred to herein as an electronic cigarette or “e-cigarette” device) to produce an aerosol vapor which is inhaled by a user. The e-liquid typically includes a base liquid, nicotine and may include a flavorant. The resulting vapor therefore also typically contains nicotine and/or a flavorant. The base liquid may include propylene glycol and/or vegetable glycerin.

A typical e-cigarette device includes a mouthpiece, a power source (typically a battery), a tank for containing e-liquid and a heating device. In use, electrical energy is supplied from the power source to the heating device, which heats the e-liquid to produce an aerosol (or “vapor”) which is inhaled by a user through the mouthpiece.

E-cigarettes can be configured in a variety of ways. For example, there are “closed system” vaping smoking substitute systems, which typically have a sealed tank and heating element. The tank is pre-filled with e-liquid and is not intended to be refilled by an end user. One subset of closed system vaping smoking substitute systems include a main body which includes the power source, wherein the main body is configured to be physically and electrically couplable to a consumable including the tank and the heating element. In this way, when the tank of a consumable has been emptied of e-liquid, that consumable is removed from the main body and disposed of. The main body can then be reused by connecting it to a new, replacement, consumable. Another subset of closed system vaping smoking substitute systems are completely disposable, and intended for one-use only.

There are also “open system” vaping smoking substitute systems which typically have a tank that is configured to be refilled by a user. In this way the entire device can be used multiple times.

An example vaping smoking substitute system is the myblu™ e-cigarette. The myblu™ e-cigarette is a closed system which includes a main body and a consumable. The main body and consumable are physically and electrically coupled together by pushing the consumable into the main body. The main body includes a rechargeable battery. The consumable includes a mouthpiece and a sealed tank which contains e-liquid. The consumable further includes a heater, which for this device is a heating filament coiled around a portion of a wick. The wick is partially immersed in the e-liquid, and conveys e-liquid from the tank to the heating filament. The system is controlled by a microprocessor on board the main body. The system includes a sensor for detecting when a user is inhaling through the mouthpiece, the microprocessor then activating the device in response. When the system is activated, electrical energy is supplied from the power source to the heating device, which heats e-liquid from the tank to produce a vapor which is inhaled by a user through the mouthpiece.

### SUMMARY OF THE DISCLOSURE

For a smoking substitute system, it is desirable to deliver nicotine into the user's lungs, where it can be absorbed into the bloodstream. However, the present disclosure is based in part on a realization that some prior art smoking substitute systems, such delivery of nicotine is not efficient. In some prior art systems, the aerosol droplets have a size distribution that is not suitable for delivering nicotine to the lungs. Aerosol droplets of a large particle size tend to be deposited in the mouth and/or upper respiratory tract. Aerosol particles of a small (e.g., sub-micron) particle size can be inhaled into the lungs but may be exhaled without delivering nicotine to the lungs. As a result, the user would require drawing a longer puff, more puffs, or vaporizing e-liquid with a higher nicotine concentration in order to achieve the desired experience.

Accordingly, there is a need for improvement in the delivery of nicotine to a user in the context of a smoking substitute system.

Development A

The present disclosure (Development A) has been devised in the light of the above considerations.

In a general aspect of Development A, the present disclosure relates to a smoking substitute device having an air passage which bypasses a vaporization chamber of the smoking substitute device.

According to a first preferred aspect of Development A there is provided a smoking substitute apparatus comprising an air inlet, a first passage leading from the air inlet to a first outlet, an aerosol generator arranged in fluid communication with the first passage, the aerosol generator being operable to generate an aerosol from an aerosol precursor, to flow in use along the first passage downstream of the aerosol generator for inhalation by a user drawing on the first outlet, wherein the apparatus further comprises a second passage leading from the air inlet to a second outlet separate from the first outlet, and wherein the second passage bypasses the first passage downstream of the aerosol generator.

Providing a smoking substitute apparatus with a second air passage separate from a first air passage allows the flow rate past or through the aerosol generator to be reduced whilst maintaining a similar overall flow rate through the apparatus. This is found to provide the benefit of increasing the average droplet size in the resultant aerosol for improved nicotine delivery to a user, while permitting the user to experience an expected inhalation rate and/or inhalation resistance.

Optionally, the first passage may comprise a vaporization chamber in which the aerosol generator is arranged, the vaporization chamber being bypassed by the second passage, and the vaporization chamber has a larger cross sectional diameter than a downstream part of the first passage.

Advantageously, the aerosol generator may comprise a heater operable to generate the aerosol from the aerosol precursor.

Conveniently, the aerosol generator may comprise a porous wick which, in use, wicks aerosol precursor from a reservoir to the first passage for entrainment in air flowing downstream of the aerosol generator.

Optionally, the heater may comprise a heating filament that is wound (e.g., helically wound) around a portion of the porous wick.

Conveniently, the part of the first passage bypassed by the second passage may comprise a flow conditioning apparatus arranged upstream of the aerosol generator which, when the smoking substitute apparatus is in use, reduces turbulence in flow at the aerosol generator. This is considered to promote the formation of larger aerosol particle sizes.

Optionally, the flow conditioning apparatus may comprise a mesh arranged in the first passage such that, in use, the flow generated by a user drawing on the first outlet passes through the mesh.

Advantageously, the first passage and the second passage may be configured such that, in use, the flow rate in the first passage is more than 1/20 of the flow rate in the second passage, preferably more than 1/10 of the flow rate in the second passage, preferably more than ⅛ of the flow rate in the second passage, preferably more than ¼ of the of the flow rate in the second passage, preferably more than ½ of the flow rate in the second passage or more than equal to the flow rate in the second passage.

Conveniently, the first passage and the second passage may be configured such that, in use, the flow rate in the first passage is less than twice of the flow rate in the second passage, preferably less than the flow rate in the second passage, preferably less than ½ of the flow rate in the second passage, preferably less than ¼ of the flow rate in the second passage, preferably less than ⅛ of the flow rate in the second passage or less than 1/10 of the flow rate in the second passage.

Optionally, in use, the d₅₀ particle size of the aerosol particles generated by the aerosol generator may be greater than 1 μm, preferably greater than 1.5 μm, or greater than 2 μm.

Advantageously, in use, the d₅₀ particle size of the aerosol particles generated by the aerosol generator may be less than 10 μm, preferably less than 8 μm, preferably less than 5 μm, preferably less than 4 μm or less than 3 μm.

Conveniently, in use, the span of particle size distribution, defined as (d₉₀−d₁₀)/d₅₀, may be less than 20, preferably less than 10, preferably less than 8, preferably less than 4, preferably less than 2, preferably less than 1, or less than 0.5.

Providing an aerosol with these particle size distribution characteristics is considered to promote the delivery of the aerosol particles to the user's lungs.

In a second aspect of Development A, a smoking substitute apparatus in accordance with any of the above statements may be comprised by or within a cartridge configured for engagement with a base unit, the cartridge and base unit together forming a smoking substitute system.

In a third aspect of Development A, a smoking substitute system may be provided, comprising a base unit and a smoking substitute apparatus according to the above statement, wherein the smoking substitute apparatus is removably engageable with the base unit.

A method is also provided for using a smoking substitute apparatus according to any one of the above aspects of Development A to generate an aerosol.

There now follows a disclosure of various optional features. These are intended to be applicable to Development A, disclosed above, and may also be applied in any combination (unless the context demands otherwise) to any aspect, embodiment or optional feature set out with respect to Development B, Development C, Development D, Development E, Development F, Development G, Development H and/or Development I.

The smoking substitute apparatus may be in the form of a consumable. The consumable may be configured for engagement with a main body. When the consumable is engaged with the main body, the combination of the consumable and the main body may form a smoking substitute system such as a closed smoking substitute system. For example, the consumable may comprise components of the system that are disposable, and the main body may comprise non-disposable or non-consumable components (e.g., power supply, controller, sensor, etc.) that facilitate the generation and/or delivery of aerosol by the consumable. In such an embodiment, the aerosol precursor (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus, with the aerosol precursor (rather than replacing a consumable component of the apparatus).

In light of this, it should be appreciated that some of the features described herein as being part of the smoking substitute apparatus may alternatively form part of a main body for engagement with the smoking substitute apparatus. This may be the case in particular when the smoking substitute apparatus is in the form of a consumable.

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting, or the like.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol precursor, such as an e-liquid. The e-liquid may, for example, comprise a base liquid. The e-liquid may further comprise nicotine. The base liquid may include propylene glycol and/or vegetable glycerin. The e-liquid may be substantially flavorless. That is, the e-liquid may not contain any deliberately added additional flavorant and may consist solely of a base liquid of propylene glycol and/or vegetable glycerin and nicotine.

The reservoir may be in the form of a tank. At least a portion of the tank may be light-transmissive. For example, the tank may comprise a window to allow a user to visually assess the quantity of e-liquid in the tank. A housing of the smoking substitute apparatus may comprise a corresponding aperture (or slot) or window that may be aligned with a light-transmissive portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage, for at least a part of the length of the passage. In this respect, the tank may surround the passage, e.g., in an annular arrangement around the passage.

The smoking substitute apparatus may comprise an aerosol generator. The aerosol generator may comprise a wick. The aerosol generator may further comprise a heater. The wick may comprise a porous material, capable of wicking the aerosol precursor. A portion of the wick may be exposed to air flow in the passage. The wick may also comprise one or more portions in contact with liquid stored in the reservoir. For example, opposing ends of the wick may protrude into the reservoir and an intermediate portion (between the ends) may extend across the passage so as to be exposed to air flow in the passage. Thus, liquid may be drawn (e.g., by capillary action) along the wick, from the reservoir to the portion of the wick exposed to air flow.

The heater may comprise a heating element, which may be in the form of a filament wound about the wick (e.g., the filament may extend helically about the wick in a coil configuration). The heating element may be wound about the intermediate portion of the wick that is exposed to air flow in the passage. The heating element may be electrically connected (or connectable) to a power source. Thus, in operation, the power source may apply a voltage across the heating element so as to heat the heating element by resistive heating. This may cause liquid stored in the wick (i.e., drawn from the tank) to be heated so as to form a vapor and become entrained in air flowing through the passage. This vapor may subsequently cool to form an aerosol in the passage, typically downstream from the heating element.

The smoking substitute apparatus may comprise a vaporization chamber. The vaporization chamber may form part of the passage in which the heater is located. The vaporization chamber may be arranged to be in fluid communication with the inlet and outlet of the passage. The vaporization chamber may be an enlarged portion of the passage. In this respect, the air as drawn in by the user may entrain the generated vapor in a flow away from heater. The entrained vapor may form an aerosol in the vaporization chamber, or it may form the aerosol further downstream along the passage. The vaporization chamber may be at least partially defined by the tank. The tank may substantially (or fully) define the vaporization chamber. In this respect, the tank may surround the vaporization chamber, e.g., in an annular arrangement around the vaporization chamber.

In use, the user may puff on a mouthpiece of the smoking substitute apparatus, i.e. draw on the smoking substitute apparatus by inhaling, to draw in an air stream therethrough. A portion, or all, of the air stream (also referred to as a “main air flow”) may pass through the vaporization chamber so as to entrain the vapor generated at the heater. That is, such a main air flow may be heated by the heater (although typically only to a limited extent) as it passes through the vaporization chamber. Alternatively, or in addition, a portion of the air stream (also referred to as a “dilution air flow” or “bypass air flow)) may bypass the vaporization chamber and be directed to mix with the generated aerosol downstream from the vaporization chamber. That is, the dilution air flow may be an air stream at an ambient temperature and may not be directly heated at all by the heater. The dilution air flow may combine with the main air flow for diluting the aerosol contained therein. The dilution air flow may merge with the main air flow along the passage downstream from the vaporization chamber. Alternatively, the dilution air flow may be directly inhaled by the user without passing though the passage of the smoking substitute apparatus.

As a user puffs on the mouthpiece, vaporized e-liquid entrained in the passing air flow may be drawn towards the outlet of the passage. The vapor may cool, and thereby nucleate and/or condense along the passage to form a plurality of aerosol droplets, e.g., nicotine-containing aerosol droplets. A portion of these aerosol droplets may be delivered to and be absorbed at a target delivery site, e.g., a user's lung, whilst a portion of the aerosol droplets may instead adhere onto other parts of the user's respiratory tract, e.g., the user's oral cavity and/or throat. Typically, in some known smoking substitute apparatuses, the aerosol droplets as measured at the outlet of the passage, e.g., at the mouthpiece, may have a droplet size, d₅₀, of less than 1 μm.

In some embodiments of the disclosure, the d₅₀ particle size of the aerosol particles is preferably at least 1 μm. Typically, the d₅₀ particle size is not more than 10 μm, preferably not more than 9 μm, not more than 8 μm, not more than 7 μm, not more than 6 μm, not more than 5 μm, not more than 4 μm or not more than 3 μm. It is considered that providing aerosol particle sizes in such ranges permits improved interaction between the aerosol particles and the user's lungs.

The particle droplet sizes, d₅₀, of an aerosol may be measured by a laser diffraction technique. For example, the stream of aerosol output from the outlet of the passage may be drawn through a Malvern Spraytec laser diffraction system, where the intensity and pattern of scattered laser light are analysed to calculate the size and size distribution of aerosol droplets. As will be readily understood, the particle size distribution may be expressed in terms of d₁₀, d₅₀ and d₉₀, for example. Considering a cumulative plot of the volume of the particles measured by the laser diffraction technique, the d₁₀ particle size is the particle size below which 10% by volume of the sample lies. The d₅₀ particle size is the particle size below which 50% by volume of the sample lies. The d₉₀ particle size is the particle size below which 90% by volume of the sample lies. Unless otherwise indicated herein, the particle size measurements are volume-based particle size measurements, rather than number-based or mass-based particle size measurements.

The spread of particle size may be expressed in terms of the span, which is defined as (d₉₀−d₁₀)/d₅₀. Typically, the span is not more than 20, preferably not more than 10, preferably not more than 8, preferably not more than 4, preferably not more than 2, preferably not more than 1, or not more than 0.5.

The smoking substitute apparatus (or main body engaged with the smoking substitute apparatus) may comprise a power source. The power source may be electrically connected (or connectable) to a heater of the smoking substitute apparatus (e.g., when the smoking substitute apparatus is engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface of the main body may be configured to transfer electrical power from the power source to a heater of the consumable via the electrical interface of the consumable.

The electrical interface of the smoking substitute apparatus may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an identification means, which may, for example, be in the form of an RFID reader, a barcode or QR code reader. This identification means may be able to identify a characteristic (e.g., a type) of a consumable engaged with the main body. In this respect, the consumable may include any one or more of an RFID chip, a barcode or QR code, or memory within which is an identifier and which can be interrogated via the identification means.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., WIFI®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may be provided in the consumable or alternatively may be provided in the main body.

The term “flavorant” is used to describe a compound or combination of compounds that provide flavor and/or aroma. For example, the flavorant may be configured to interact with a sensory receptor of a user (such as an olfactory or taste receptor). The flavorant may include one or more volatile substances.

The flavorant may be provided in solid or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations.

The present inventors consider that a flow rate of 1.3 L min⁻¹ is towards the lower end of a typical user expectation of flow rate through a conventional cigarette and therefore through a user-acceptable smoking substitute apparatus. The present inventors further consider that a flow rate of 2.0 L min⁻¹ is towards the higher end of a typical user expectation of flow rate through a conventional cigarette and therefore through a user-acceptable smoking substitute apparatus. Embodiments of the present disclosure therefore provide an aerosol with advantageous particle size characteristics across a range of flow rates of air through the apparatus.

The aerosol may have a Dv50 of at least 1.1 μm, at least 1.2 μm, at least 1.3 μm, at least 1.4 μm, at least 1.5 μm, at least 1.6 μm, at least 1.7 μm, at least 1.8 μm, at least 1.9 μm or at least 2.0 μm.

The aerosol may have a Dv50 of not more than 4.9 μm, not more than 4.8 μm, not more than 4.7 μm, not more than 4.6 μm, not more than 4.5 μm, not more than 4.4 μm, not more than 4.3 μm, not more than 4.2 μm, not more than 4.1 μm, not more than 4.0 μm, not more than 3.9 μm, not more than 3.8 μm, not more than 3.7 μm, not more than 3.6 μm, not more than 3.5 μm, not more than 3.4 μm, not more than 3.3 μm, not more than 3.2 μm, not more than 3.1 μm or not more than 3.0 μm.

A particularly preferred range for Dv50 of the aerosol is in the range 2-3 μm.

The air inlet, flow passage, outlet and the vaporization chamber may be configured so that, when the air flow rate inhaled by the user through the apparatus is 1.3 L min′, the average magnitude of velocity of air in the vaporization chamber is in the range 0-1.3 ms⁻¹. The average magnitude velocity of air may be calculated based on knowledge of the geometry of the vaporization chamber and the flow rate.

When the air flow rate inhaled by the user through the apparatus is 1.3 L min⁻¹, the average magnitude of velocity of air in the vaporization chamber may be at least 0.001 ms⁻¹, or at least 0.005 ms⁻¹, or at least 0.01 ms⁻¹, or at least 0.05 ms⁻¹.

When the air flow rate inhaled by the user through the apparatus is 1.3 L min⁻¹, the average magnitude of velocity of air in the vaporization chamber may be at most 1.2 ms⁻¹, at most 1.1 ms⁻¹, at most 1.0 ms⁻¹, at most 0.9 ms⁻¹, at most 0.8 ms⁻¹, at most 0.7 ms⁻¹ or at most 0.6 ms⁻¹.

The air inlet, flow passage, outlet and the vaporization chamber may be configured so that, when the air flow rate inhaled by the user through the apparatus is 2.0 L min⁻¹, the average magnitude of velocity of air in the vaporization chamber is in the range 0-1.3 ms⁻¹. The average magnitude velocity of air may be calculated based on knowledge of the geometry of the vaporization chamber and the flow rate.

When the air flow rate inhaled by the user through the apparatus is 2.0 L min⁻¹, the average magnitude of velocity of air in the vaporization chamber may be at least 0.001 ms⁻¹, or at least 0.005 ms⁻¹, or at least 0.01 ms⁻¹, or at least 0.05 ms⁻¹.

When the air flow rate inhaled by the user through the apparatus is 2.0 L min⁻¹, the average magnitude of velocity of air in the vaporization chamber may be at most 1.2 ms⁻¹, at most 1.1 ms⁻¹, at most 1.0 ms⁻¹, at most 0.9 ms⁻¹, at most 0.8 ms⁻¹, at most 0.7 ms⁻¹ or at most 0.6 ms⁻¹.

When the calculated average magnitude of velocity of air in the vaporization chamber is in the ranges specified, it is considered that the resultant aerosol particle size is advantageously controlled to be in a desirable range. It is further considered that the configuration of the apparatus can be selected so that the average magnitude of velocity of air in the vaporization chamber can be brought within the ranges specified, at the exemplary flow rate of 1.3 L min⁻¹ and/or the exemplary flow rate of 2.0 L min⁻¹.

The aerosol generator may comprise a vaporizer element loaded with aerosol precursor, the vaporizer element being heatable by a heater and presenting a vaporizer element surface to air in the vaporization chamber. A vaporizer element region may be defined as a volume extending outwardly from the vaporizer element surface to a distance of 1 mm from the vaporizer element surface.

The air inlet, flow passage, outlet and the vaporization chamber may be configured so that, when the air flow rate inhaled by the user through the apparatus is 1.3 L min⁻¹, the average magnitude of velocity of air in the vaporizer element region is in the range 0-1.2 ms⁻¹. The average magnitude of velocity of air in the vaporizer element region may be calculated using computational fluid dynamics.

When the air flow rate inhaled by the user through the apparatus is 1.3 L min⁻¹, the average magnitude of velocity of air in the vaporizer element region may be at least 0.001 ms⁻¹, or at least 0.005 ms⁻¹, or at least 0.01 ms⁻¹, or at least 0.05 ms⁻¹.

When the air flow rate inhaled by the user through the apparatus is 1.3 L min′, the average magnitude of velocity of air in the vaporizer element region may be at most 1.1 ms⁻¹, at most 1.0 ms⁻¹, at most 0.9 ms⁻¹, at most 0.8 ms⁻¹, at most 0.7 ms⁻¹ or at most 0.6 ms⁻¹.

The air inlet, flow passage, outlet and the vaporization chamber may be configured so that, when the air flow rate inhaled by the user through the apparatus is 2.0 L min⁻¹, the average magnitude of velocity of air in the vaporizer element region is in the range 0-1.2 ms⁻¹. The average magnitude of velocity of air in the vaporizer element region may be calculated using computational fluid dynamics.

When the air flow rate inhaled by the user through the apparatus is 2.0 L min⁻¹, the average magnitude of velocity of air in the vaporizer element region may be at least 0.001 ms⁻¹, or at least 0.005 ms⁻¹, or at least 0.01 ms⁻¹, or at least 0.05 ms⁻¹.

When the air flow rate inhaled by the user through the apparatus is 2.0 L min⁻¹, the average magnitude of velocity of air in the vaporizer element region may be at most 1.1 ms⁻¹, at most 1.0 ms⁻¹, at most 0.9 ms⁻¹, at most 0.8 ms⁻¹, at most 0.7 ms⁻¹ or at most 0.6 ms⁻¹.

When the average magnitude of velocity of air in the vaporizer element region is in the ranges specified, it is considered that the resultant aerosol particle size is advantageously controlled to be in a desirable range. It is further considered that the velocity of air in the vaporizer element region is more relevant to the resultant particle size characteristics than consideration of the velocity in the vaporization chamber as a whole. This is in view of the significant effect of the velocity of air in the vaporizer element region on the cooling of the vapor emitted from the vaporizer element surface.

Additionally, or alternatively is it relevant to consider the maximum magnitude of velocity of air in the vaporizer element region.

Therefore, the air inlet, flow passage, outlet and the vaporization chamber may be configured so that, when the air flow rate inhaled by the user through the apparatus is 1.3 L min⁻¹, the maximum magnitude of velocity of air in the vaporizer element region is in the range 0-2.0 ms⁻¹.

When the air flow rate inhaled by the user through the apparatus is 1.3 L min⁻¹, the maximum magnitude of velocity of air in the vaporizer element region may be at least 0.001 ms⁻¹, or at least 0.005 ms⁻¹, or at least 0.01 ms⁻¹, or at least 0.05 ms⁻¹.

When the air flow rate inhaled by the user through the apparatus is 1.3 L min⁻¹, the maximum magnitude of velocity of air in the vaporizer element region may be at most 1.9 ms⁻¹, at most 1.8 ms⁻¹, at most 1.7 ms⁻¹, at most 1.6 ms⁻¹, at most 1.5 ms⁻¹, at most 1.4 ms⁻¹, at most 1.3 ms⁻¹ or at most 1.2 ms⁻¹.

The air inlet, flow passage, outlet and the vaporization chamber may be configured so that, when the air flow rate inhaled by the user through the apparatus is 2.0 L min⁻¹, the maximum magnitude of velocity of air in the vaporizer element region is in the range 0-2.0 ms⁻¹.

When the air flow rate inhaled by the user through the apparatus is 2.0 L min⁻¹, the maximum magnitude of velocity of air in the vaporizer element region may be at least 0.001 ms⁻¹, or at least 0.005 ms⁻¹, or at least 0.01 ms⁻¹, or at least 0.05 ms⁻¹.

When the air flow rate inhaled by the user through the apparatus is 2.0 L min⁻¹, the maximum magnitude of velocity of air in the vaporizer element region may be at most 1.9 ms⁻¹, at most 1.8 ms⁻¹, at most 1.7 ms⁻¹, at most 1.6 ms⁻¹, at most 1.5 ms⁻¹, at most 1.4 ms⁻¹, at most 1.3 ms⁻¹ or at most 1.2 ms⁻¹.

It is considered that configuring the apparatus in a manner to permit such control of velocity of the airflow at the vaporizer permits the generation of aerosols with particularly advantageous particle size characteristics, including Dv50 values.

Additionally, or alternatively is it relevant to consider the turbulence intensity in the vaporizer chamber in view of the effect of turbulence on the particle size of the generated aerosol. For example, the air inlet, flow passage, outlet and the vaporization chamber may be configured so that, when the air flow rate inhaled by the user through the apparatus is 1.3 L min⁻¹, the turbulence intensity in the vaporizer element region is not more than 1%.

When the air flow rate inhaled by the user through the apparatus is 1.3 L min⁻¹, the turbulence intensity in the vaporizer element region may be not more than 0.95%, not more than 0.9%, not more than 0.85%, not more than 0.8%, not more than 0.75%, not more than 0.7%, not more than 0.65% or not more than 0.6%.

It is considered that configuring the apparatus in a manner to permit such control of the turbulence intensity in the vaporizer element region permits the generation of aerosols with particularly advantageous particle size characteristics, including Dv50 values.

Following detailed investigations, the inventors consider, without wishing to be bound by theory, that the particle size characteristics of the generated aerosol may be determined by the cooling rate experienced by the vapor after emission from the vaporizer element (e.g., wick). In particular, it appears that imposing a relatively slow cooling rate on the vapor has the effect of generating aerosols with a relatively large particle size. The parameters discussed above (velocity and turbulence intensity) are considered to be mechanisms for implementing a particular cooling dynamic to the vapor.

More generally, it is considered that the air inlet, flow passage, outlet and the vaporization chamber may be configured so that a desired cooling rate is imposed on the vapor. The particular cooling rate to be used depends of course on the nature of the aerosol precursor and other conditions. However, for a particular aerosol precursor it is possible to define a set of testing conditions in order to define the cooling rate, and by extension this imposes limitations on the configuration of the apparatus to permit such cooling rates as are shown to result in advantageous aerosols. Accordingly, the air inlet, flow passage, outlet and the vaporization chamber may be configured so that the cooling rate of the vapor is such that the time taken to cool to 50° C. is not less than 16 ms, when tested according to the following protocol. The aerosol precursor is an e-liquid consisting of 1.6% freebase nicotine and the remainder a 65:35 propylene glycol and vegetable glycerin mixture, the e-liquid having a boiling point of 209° C. Air is drawn into the air inlet at a temperature of 25° C. The vaporizer is operated to release a vapor of total particulate mass 5 mg over a 3 second duration from the vaporizer element surface in an air flow rate between the air inlet and outlet of 1.3 L min⁻¹.

Additionally, or alternatively, the air inlet, flow passage, outlet and the vaporization chamber may be configured so that the cooling rate of the vapor is such that the time taken to cool to 50° C. is not less than 16 ms, when tested according to the following protocol. The aerosol precursor is an e-liquid consisting of 1.6% freebase nicotine and the remainder a 65:35 propylene glycol and vegetable glycerin mixture, the e-liquid having a boiling point of 209° C. Air is drawn into the air inlet at a temperature of 25° C. The vaporizer is operated to release a vapor of total particulate mass 5 mg over a 3 second duration from the vaporizer element surface in an air flow rate between the air inlet and outlet of 2.0 L min⁻¹.

Cooling of the vapor such that the time taken to cool to 50° C. is not less than 16 ms corresponds to an equivalent linear cooling rate of not more than 10° C./ms.

The equivalent linear cooling rate of the vapor to 50° C. may be not more than 9° C./ms, not more than 8° C./ms, not more than 7° C./ms, not more than 6° C./ms or not more than 5° C./ms.

Cooling of the vapor such that the time taken to cool to 50° C. is not less than 32 ms corresponds to an equivalent linear cooling rate of not more than 5° C./ms.

The testing protocol set out above considers the cooling of the vapor (and subsequent aerosol) to a temperature of 50° C. This is a temperature which can be considered to be suitable for an aerosol to exit the apparatus for inhalation by a user without causing significant discomfort. It is also possible to consider cooling of the vapor (and subsequent aerosol) to a temperature of 75° C. Although this temperature is possibly too high for comfortable inhalation, it is considered that the particle size characteristics of the aerosol are substantially settled by the time the aerosol cools to this temperature (and they may be settled at still higher temperature).

Accordingly, the air inlet, flow passage, outlet and the vaporization chamber may be configured so that the cooling rate of the vapor is such that the time taken to cool to 75° C. is not less than 4.5 ms, when tested according to the following protocol. The aerosol precursor is an e-liquid consisting of 1.6% freebase nicotine and the remainder a 65:35 propylene glycol and vegetable glycerin mixture, the e-liquid having a boiling point of 209° C. Air is drawn into the air inlet at a temperature of 25° C. The vaporizer is operated to release a vapor of total particulate mass 5 mg over a 3 second duration from the vaporizer element surface in an air flow rate between the air inlet and outlet of 1.3 L min⁻¹.

Additionally, or alternatively, the air inlet, flow passage, outlet and the vaporization chamber may be configured so that the cooling rate of the vapor is such that the time taken to cool to 75° C. is not less than 4.5 ms, when tested according to the following protocol. The aerosol precursor is an e-liquid consisting of 1.6% freebase nicotine and the remainder a 65:35 propylene glycol and vegetable glycerin mixture, the e-liquid having a boiling point of 209° C. Air is drawn into the air inlet at a temperature of 25° C. The vaporizer is operated to release a vapor of total particulate mass 5 mg over a 3 second duration from the vaporizer element surface in an air flow rate between the air inlet and outlet of 2.0 L min⁻¹.

Cooling of the vapor such that the time taken to cool to 75° C. is not less than 4.5 ms corresponds to an equivalent linear cooling rate of not more than 30° C./ms.

The equivalent linear cooling rate of the vapor to 75° C. may be not more than 29° C./ms, not more than 28° C./ms, not more than 27° C./ms, not more than 26° C./ms, not more than 25° C./ms, not more than 24° C./ms, not more than 23° C./ms, not more than 22° C./ms, not more than 21° C./ms, not more than 20° C./ms, not more than 19° C./ms, not more than 18° C./ms, not more than 17° C./ms, not more than 16° C./ms, not more than 15° C./ms, not more than 14° C./ms, not more than 13° C./ms, not more than 12° C./ms, not more than 11° C./ms or not more than 10° C./ms.

Cooling of the vapor such that the time taken to cool to 75° C. is not less than 13 ms corresponds to an equivalent linear cooling rate of not more than 10° C./ms.

It is considered that configuring the apparatus in a manner to permit such control of the cooling rate of the vapor permits the generation of aerosols with particularly advantageous particle size characteristics, including Dv50 values.

Development B

For a smoking substitute system, it is desirable to deliver nicotine into the user's lungs, where it can be absorbed into the bloodstream. However, the present disclosure is based in part on a realization that some prior art smoking substitute systems, such delivery of nicotine is not efficient. In some prior art systems, the aerosol droplets have a size distribution that is not suitable for delivering nicotine to the lungs. Aerosol droplets of a large particle size tend to be deposited in the mouth and/or upper respiratory tract. Aerosol particles of a small (e.g., sub-micron) particle size can be inhaled into the lungs but may be exhaled without delivering nicotine to the lungs. As a result, the user would require drawing a longer puff, more puffs, or vaporizing e-liquid with a higher nicotine concentration in order to achieve the desired experience.

The present disclosure is also devised to ameliorate a problem associated with the generation of heat in some smoking substitute systems. Namely, when a heating device heats an aerosol precursor, the enclosure which surrounds the heater and aerosol precursor is also subjected to heating. When unshielded from the heating device, the enclosure can undergo thermal degradation, such as melting, softening, corrosion, spalling or combustion, which can result in deformation of the enclosure, and/or unwanted materials being entrained in the air flow for inhalation by a user. This phenomenon is considered to be especially likely to occur when the enclosure is made of a plastics material. It is possible to mitigate this risk by designing relatively large and bulky smoking substitute systems in which the enclosure is disposed far away from the heater. Such systems are less convenient to store and hold by a user due to their size, and more costly to manufacture due to the large amount of material used to make them.

Accordingly, there is a need for improvement in the performance of a smoking substitute system while still ensuring suitable delivery of nicotine to a user.

The present disclosure (Development B) has been devised in the light of the above considerations.

In a general aspect of Development B, the present disclosure relates to a smoking substitute system providing an enclosure which defines a vaporization chamber, an air flow which at least partially bypasses the vaporization chamber in use, and a heat shield provided between a part of that enclosure made from plastics material and a heater of an aerosol generator that is at least partially enclosed by the enclosure.

According to a first preferred aspect of Development B there is provided a smoking substitute apparatus comprising: an air inlet and an outlet; an aerosol generator; an enclosure defining a vaporization chamber, wherein the enclosure at least partially encloses the aerosol generator, wherein the aerosol generator comprises a heater, the aerosol generator being operable to generate an aerosol by vaporizing an aerosol precursor, the aerosol being for entrainment in an air flow flowing in use from the air inlet to the outlet when a user draws air through the apparatus, wherein, in use, at least a part of the air flow from the air inlet to the outlet bypasses the vaporization chamber, and wherein at least part of the enclosure adjacent the heater is formed from plastics material, there being provided a heat shield between said part of the enclosure and the heater in the vaporization chamber.

In use of the apparatus of the first aspect of Development B, the heater generates heat energy. Some of this energy heats and vaporizes the aerosol precursor, with the remainder of the heat energy being excess heat energy. Some of the excess heat energy, in the absence of the present disclosure, would disadvantageously heat the part of the enclosure formed of plastics material. Advantageously, the heat shield, which is provided between the heater and said part of the enclosure, intercepts and absorbs a significant proportion of the excess heat directed towards said part of the enclosure and reduces heating thereof. This reduces the risk of thermal degradation of said part of the enclosure. Note that, because a significant part of the air flow may bypass the vaporization chamber, the typical cooling effect of the bypass airflow through the vaporization chamber is not provided. Therefore, the material of the enclosure is particularly at risk of thermal degradation in the absence of the features of the present disclosure. Additionally, the aerosol generator and the enclosure of the apparatus can be positioned closer together than in a corresponding case in which the heat shield is absent.

Optionally, the apparatus may further comprise a housing disposed externally of the enclosure, the housing being formed at least in part from a plastics material. Having a housing formed of a plastics material allows the apparatus to be light-weight and cheap to manufacture. In such an apparatus, the heat shield reduces the risk of thermal degradation of the housing caused by the heater.

Conveniently, the closest distance between said part of the enclosure and the heater, may be at most 2 mm. This allows the overall size of the apparatus to be kept low, for user convenience, but the incorporation of the features of the disclosure reduces the risk of thermal degradation of the enclosure. More preferably, the closest distance between said part of the enclosure and the heater may be at most 1.5 mm, or at most 1 mm. Alternatively or additionally, the closest distance between said part of the enclosure and the heater may be at least 0.25 mm, or at least 0.5 mm. This reduces a risk of thermal degradation of the enclosure due to a close proximity to the heater.

Advantageously, the heat shield may be formed of a material having a thermal degradation temperature at least 100° C. higher than that of the plastics material forming the enclosure. This reduces a risk of the heat shield thermally degrading and allowing more heat energy to be absorbed by the enclosure. More preferably, the thermal degradation temperature may be at least 150° C. higher than that of the plastics material. More preferably the thermal degradation temperature may be at least 200° C. higher than that of the plastics material.

The heat shield may be formed of a material selected from metals, thermosetting polymers and ceramics and composites thereof. Such materials are resistant to thermal degradation, and thus the risk of failure of such heat shields is low.

The heat shield may present to the heater a heat-absorbing surface having an area of at least twice as large as a plan view projection of the heater onto the heatshield. This ensures that heat radiating from the heater does not easily bypass the heat shield and excessively heat the enclosure. The area of the heat-absorbing surface of the heat shield may be at least 20 mm², or more preferably at least 30 mm², or more preferably at least 40 mm².

The vaporization chamber may have an elongate shape, wider in a first direction orthogonal to a longitudinal axis of the apparatus compared with a second direction orthogonal to the first direction and to the longitudinal axis of the apparatus, the heater extending along the first direction and the heat shield extending between the heater and the enclosure substantially parallel to the first direction. The wider first direction allows a sufficiently large heater to be provided for the vaporization of the aerosol precursor, while the narrower second direction ensures the apparatus is not too bulky for a user to hold.

There may be provided first and second heat shield plates disposed on opposing sides of the heater. Such plates provide protection to the enclosure on opposing sides of the heater, without significantly increasing the size of the apparatus.

In use, substantially all of the air flow from the air inlet to the outlet may bypass the vaporization chamber, the vaporization chamber having a vaporization chamber outlet in communication with a passage along which air flows from the air inlet to the outlet. This allows particles of aerosol generated by vaporization of the aerosol precursor to grow to a suitable size prior to becoming entrained in the air flow and inhaled by a user. This provides more efficient nicotine delivery to the user. The vaporization chamber may be substantially sealed against air flow except for the vaporization chamber outlet. This further ensures that the particles of the aerosol enter the air flow at substantially the same point in time, ensuring a more homogenous distribution of particle sizes in the air flow.

A first passage may lead from the air inlet to the outlet, the aerosol generator being arranged in fluid communication with the first passage, the apparatus may further comprise a second passage leading from the air inlet (or from a second air inlet) to the outlet, wherein the second passage bypasses the first passage downstream of the aerosol generator. This allows the rate of air flow over the aerosol generator to be maintained at a level low enough to entrain sufficiently sized aerosol particles, even for a user with a high inhalation flow rate.

The smoking substitute apparatus may be comprised by or within a cartridge configured for engagement with a base unit, the cartridge and base unit together forming a smoking substitute system.

According to a second preferred aspect of Development B, there is provided a smoking substitute system comprising: a base unit, and a smoking substitute apparatus according to the first preferred aspect wherein the smoking substitute apparatus is comprised by or within a cartridge configured for engagement with a base unit, the cartridge and base unit together forming a smoking substitute system, wherein the smoking substitute apparatus is removably engageable with the base unit.

According to a third preferred aspect of Development B, there is provided a method of using a smoking substitute apparatus according to the first preferred aspect of Development B to generate an aerosol.

Development C

For a smoking substitute system, it is desirable to deliver nicotine into the user's lungs, where it can be absorbed into the bloodstream. However, the present disclosure is based in part on a realization that some prior art smoking substitute systems, such delivery of nicotine is not efficient. In some prior art systems, the aerosol droplets have a size distribution that is not suitable for delivering nicotine to the lungs. Aerosol droplets of a large particle size tend to be deposited in the mouth and/or upper respiratory tract. Aerosol particles of a small (e.g., sub-micron) particle size can be inhaled into the lungs but may be exhaled without delivering nicotine to the lungs. As a result, the user would require drawing a longer puff, more puffs, or vaporizing e-liquid with a higher nicotine concentration in order to achieve the desired experience.

Based on the insight of the present inventors, it is considered that a factor associated with generating suitable aerosol droplet size distributions in typical smoking substitute systems is the velocity of air flowing over a vaporizer (or more generally an aerosol generator) that generates aerosol droplets. In some prior art systems, a single air flow passage extends between an air inlet and a mouthpiece with a vaporizer disposed in the passage. When a user inhales at the mouthpiece, an air flow is generated in the passage that passes over the vaporizer. As there is only one passage through which the air can flow, the air velocity over the vaporizer is determined by the inhalation flow rate of the user. As such, if the user inhales too vigorously, the air velocity over the vaporizer becomes too large to generate aerosol droplets of a suitable size distribution, thus lowering the efficiency of nicotine delivery for the system.

Accordingly, there is a need for improvement in the delivery of nicotine to a user in the context of a smoking substitute system.

The present disclosure (Development C) has been devised in the light of the above considerations.

In a general aspect of Development C, the present disclosure provides a bypass flow channel, permitting division of an air flow derived from an air inlet between the bypass flow channel and a main flow channel, air flow in the bypass flow channel flowing in an opposing direction to air flow in at least a part of the main flow channel.

According to a first preferred aspect of Development C there is provided a smoking substitute apparatus comprising:

a housing having a first end and a second end, and at least one sidewall:
an air inlet provided at said sidewall of the housing;
an outlet provided at the second end of the housing;
a main flow channel and a bypass flow channel extending in different directions from the air inlet, the main flow channel and the bypass flow channel being in communication with the outlet;
an aerosol generator positioned in the main flow channel for generating aerosol from an aerosol precursor;
wherein in use, air flowing through the air inlet is split into a main air flow and a bypass air flow, the main and bypass air flows flowing along the main flow channel and the bypass flow channel respectively, wherein immediately downstream of the air inlet, the main air flow travels towards one of the first and second ends of the housing, and the bypass air flow travels towards the other of the first and second ends of the housing.

Advantageously, the arrangement of the main and bypass flow channels allows a flow demanded at the outlet (typically caused by a user drawing air through the apparatus, e.g., by inhalation) to be shared between the main and bypass flow channels. Hence, when a user inhales from the apparatus, the rate of flow in the main channel, and hence the air velocity over the aerosol generator, is lower than a corresponding case where the bypass channel is absent. This is useful in cases where the user inhales with a high flow rate, as it prevents the air velocity over the aerosol generator from becoming too high to generate suitably sized aerosol droplets.

This difference in air flow direction along the main and bypass flow channels from the air inlet is conducive to the main and bypass flow channels having different lengths. Such a difference causes a difference in flow resistance between the channels, which for a given apparatus can be tuned to tailor the ratio of flow between the main flow channel and the bypass flow channel. Hence, the apparatus can be tailored to the inhalation flow rates of an individual user or group of users.

Additionally, providing the air inlet at a sidewall of the housing reduces the probability of aerosol precursor and/or condensed aerosol from leaking out of the housing.

In use, immediately downstream of the air inlet, the main air flow may travel towards the first end of the housing, and the bypass air flow may travel towards the second end of the housing.

The aerosol generator may include a heater. The aerosol precursor may be a liquid. Thus, the aerosol generator may operate to vaporize the aerosol precursor using heat generated by the heater, the vapor subsequently forming an aerosol entrained in air flowing along the main channel.

Advantageously, the air inlet may be disposed substantially at a midpoint of the sidewall.

Optionally, the air inlet is defined by a T-junction between an opening in the sidewall of the housing and the main and bypass flow channels.

Conveniently, downstream of the aerosol generator, the main air flow may be in the opposite direction to the main air flow upstream of the aerosol generator and proximate the air inlet.

Advantageously, the smoking substitute apparatus of the first aspect may be configured to generate an aerosol having a median droplet size, d₅₀, of at least 1 μm.

According to a second aspect of Development C there is provided a smoking substitute device configured to engage with the smoking substitute apparatus of the first aspect of Development C, wherein the device comprises a controller and a power source configured to energize the aerosol generator.

According to a third aspect of Development C there is provided a smoking substitute system for generating an aerosol, comprising: the smoking substitute apparatus of the first aspect of Development C; and the smoking substitute device of the second aspect of Development C.

According to a fourth aspect of Development C there is provided a method of generating an aerosol using the smoking substitute apparatus of the first aspect of Development C, wherein the droplets of the aerosol have a median diameter, d₅₀, of at least 1 μm.

Development D

For a smoking substitute system, it is desirable to deliver nicotine into the user's lungs, where it can be absorbed into the bloodstream. However, the present disclosure is based in part on a realization that in some prior art smoking substitute systems, such delivery of nicotine is not efficient. In some prior art systems, the aerosol droplets have a size distribution that is not suitable for delivering nicotine to the lungs. Aerosol droplets of a large particle size tend to be deposited in the mouth and/or upper respiratory tract. Aerosol particles of a small (e.g., sub-micron) particle size can be inhaled into the lungs but may be exhaled without delivering nicotine to the lungs. As a result, the user would require drawing a longer puff, more puffs, or vaporizing e-liquid with a higher nicotine concentration in order to achieve the desired experience.

Accordingly, there is a need for improvement in the delivery of nicotine to a user in the context of a smoking substitute system.

The present disclosure (Development D) has been devised in the light of the above considerations.

In a general aspect of Development D, the present disclosure relates to a smoking substitute apparatus having a main air channel and a bypass air channel wherein the apparatus comprises an auxiliary heater for heating air in the bypass air channel.

According to a first preferred aspect of Development D there is provided a smoking substitute apparatus comprising one or more air inlets, one or more outlets, a first passage leading from at least one of the air inlets to a first outlet among the outlets, an aerosol generator arranged in a vaporization chamber in the first passage, the aerosol generator comprising a heater and being operable to generate an aerosol from an aerosol precursor to flow in use along the first passage downstream of the aerosol generator for inhalation by a user drawing on the first outlet, and a second passage leading from at least one of the air inlets to at least one of the outlets wherein the second passage bypasses the vaporization chamber of the first passage. The second passage comprises an auxiliary heater for heating air in the second passage.

Providing an auxiliary heater for heating air in the second passage may reduce adverse effects on the characteristics of the aerosol (e.g., average droplet size, d₅₀) arising from any cooling effect from the air flow in the second passage.

Optionally, the vaporization chamber may have a larger cross sectional diameter than a downstream part of the first passage.

Advantageously, the auxiliary heater for heating the air in the second passage may comprise an electrically heatable mesh.

Conveniently, the auxiliary heater for heating the air in the second passage may comprise an electrically heatable heating coil.

Optionally, the auxiliary heater for heating the air in the second passage may comprise an electrically heatable heating plate.

Advantageously, the auxiliary heater for heating the air in the second passage may be heatable by resistive heating using an electrical current.

Conveniently, the auxiliary heater for heating the air in the second passage may comprise a thermally conductive element in thermal communication with the heater of the aerosol generator.

Optionally, the auxiliary heater for heating the air in the second passage may be heatable independently from the heater in the first passage.

Advantageously, the auxiliary heater for heating the air in the second passage may be heatable in combination with the heater in the first passage.

Conveniently, the auxiliary heater for heating the air in the second passage may be heatable in synchronism with the heater in the first passage.

Optionally, the smoking substitute apparatus may comprise an air inlet to the second passage which is not spatially coterminous with an air inlet to the first passage.

Advantageously, the smoking substitute apparatus may comprise an air outlet from the second passage which is not spatially coterminous with an air outlet from the first passage.

Conveniently, the part of the first passage bypassed by the second passage may comprise a flow conditioning apparatus arranged upstream of the aerosol generator which, when the smoking substitute apparatus is in use, reduces turbulence in flow at the aerosol generator.

Optionally, the flow conditioning apparatus may comprise a mesh arranged in the first passage such that, in use, the flow generated by a user drawing on the first outlet passes through the mesh.

Advantageously, the first passage and the second passage may be configured such that, in use, the flow rate in the first passage is more than 1/20 of the flow rate in the second passage.

Conveniently, the first passage and the second passage may be configured such that, in use, the flow rate in the first passage is less than twice of the flow rate in the second passage.

Optionally, in use, the d₅₀ particle size of the aerosol particles generated by the aerosol generator may be greater than 1 μm.

Advantageously, in use, the d₅₀ particle size of the aerosol particles generated by the aerosol generator may be less than 10 μm.

Conveniently, in use, the span of particle size distribution, defined as (d₉₀−d₁₀)/d₅₀, may be less than 20.

In another aspect of Development D, the smoking substitute apparatus is comprised by or within a cartridge configured for engagement with a base unit, the cartridge and base unit together forming a smoking substitute system.

In a third aspect of Development D, a smoking substitute apparatus is provided, comprising a base unit, and a smoking substitute apparatus, wherein the smoking substitute apparatus is removably engageable with the base unit.

In a fourth aspect of Development D, a method may be provided for using a smoking substitute apparatus according to the first, second or third aspects of Development D to generate an aerosol.

Development E

For a smoking substitute system, it is desirable to deliver nicotine into the user's lungs, where it can be absorbed into the bloodstream. However, the present disclosure is based in part on a realization that some prior art smoking substitute systems, such delivery of nicotine is not efficient. In some prior art systems, the aerosol droplets have a size distribution that is not suitable for delivering nicotine to the lungs. Aerosol droplets of a large particle size tend to be deposited in the mouth and/or upper respiratory tract. Aerosol particles of a small (e.g., sub-micron) particle size can be inhaled into the lungs but may be exhaled without delivering nicotine to the lungs. As a result, the user would require drawing a longer puff, more puffs, or vaporizing e-liquid with a higher nicotine concentration in order to achieve the desired experience.

Accordingly, there is a need for improvement in the delivery of nicotine to a user in the context of a smoking substitute system.

The present disclosure (Development E) has been devised in the light of the above considerations.

In a general aspect of Development E, the present disclosure relates to a smoking substitute apparatus having a main passage and a bypass passage wherein the bypass passage joins with the main passage at a junction proximate to the outlet of the main passage and in a substantially perpendicular direction to the main passage.

According to a first preferred aspect of Development E there is provided a smoking substitute apparatus comprising an air inlet, a vaporizer passage leading from the air inlet to an outlet, an aerosol generator arranged in a vaporization chamber in the vaporizer passage, the aerosol generator being operable to generate an aerosol from an aerosol precursor to flow in use along the vaporizer passage downstream of the aerosol generator for inhalation by a user drawing on the apparatus and a bypass passage leading from the air inlet to a bypass passage outlet at a junction arranged adjacent to the outlet, wherein the bypass passage bypasses the vaporization chamber of the vaporizer passage, The bypass passage meets the vaporizer passage at the junction in a direction substantially perpendicular to a longitudinal axis of the vaporizer passage at the junction.

Providing a vaporizer passage and a bypass passage that join at a junction in a substantially perpendicular direction may improve mixing between the air in the two passages to provide a more consistent aerosol vapor to a user.

Optionally, the smoking substitute apparatus may comprise a plurality of bypass passages, each extending from the air inlet to the junction, wherein a longitudinal axis of at least one of the bypass passage outlets at the junction is offset from the longitudinal axis of the vaporizer passage at the junction.

Conveniently, the smoking substitute apparatus may comprise a plurality of bypass passages, each extending from the air inlet to a respective bypass passage outlet at the junction, wherein a first bypass passage outlet at the junction is offset from a second bypass passage outlet of the bypass passage outlets at the junction along a direction substantially parallel to the longitudinal axis of the vaporizer passage at the junction.

Advantageously, the bypass passage outlets at the junction may be arranged such that, in use, air drawn through the bypass passages generates a vortex at or near the junction in air drawn through the vaporizer passage.

Optionally, the bypass passage outlets at the junction may be arranged such that, in use, air drawn through the bypass passages generates a plurality of counter-rotating vortices proximate to the junction in air drawn through the vaporizer passage.

Conveniently, the smoking substitute apparatus may comprise a plurality of bypass passages, each extending from the air inlet to the junction, wherein at least one pair of the bypass passage outlets are arranged at symmetrically opposed positions across the vaporizer passage.

Advantageously, the aerosol generator may comprise a heater operable to generate the aerosol from the aerosol precursor.

Optionally, the aerosol generator may comprise a porous wick which, in use, wicks aerosol precursor from a reservoir to the vaporizer passage for entrainment in air flowing downstream of the aerosol generator.

Conveniently, the heater may comprise a heating filament that is wound around a portion of the porous wick.

Advantageously, the part of the vaporizer passage bypassed by the bypass passage may comprise a flow conditioning apparatus arranged upstream of the aerosol generator which, when the smoking substitute apparatus is in use, reduces turbulence in flow at the aerosol generator.

Optionally, the flow conditioning apparatus may comprise a mesh arranged in the vaporizer passage such that, in use, the flow generated by a user drawing on the outlet passes through the mesh.

Conveniently, the vaporizer passage and the bypass passage may be configured such that, in use, the flow rate in the vaporizer passage is more than 1/20 of the flow rate in the bypass passage.

Advantageously, the vaporizer passage and the bypass passage may be configured such that, in use, the flow rate in the vaporizer passage is less than twice of the flow rate in the bypass passage.

Optionally, in use, the d₅₀ particle size of the aerosol particles generated by the aerosol generator may be greater than 1 μm.

Conveniently, in use, the d₅₀ particle size of the aerosol particles generated by the aerosol generator may be less than 10 μm.

Advantageously, in use, the span of particle size distribution, defined as (d₉₀−d₁₀)/d₅₀, may be less than 20.

In another aspect of Development E, the smoking substitute apparatus is comprised by or within a cartridge configured for engagement with a base unit, the cartridge and base unit together forming a smoking substitute system.

In a third aspect of Development E, a smoking substitute system is provided, the smoking substitute system comprising a base unit, and a smoking substitute apparatus, wherein the smoking substitute apparatus is removably engageable with the base unit.

In a fourth aspect of Development E, a method of using the smoking substitute apparatus of the first, second or third aspects of Development E to generate an aerosol is provided.

Development F

For a smoking substitute system, it is desirable to deliver nicotine into the user's lungs, where it can be absorbed into the bloodstream. However, the present disclosure is based in part on a realization that some prior art smoking substitute systems, such delivery of nicotine is not efficient. In some prior art systems, the aerosol droplets have a size distribution that is not suitable for delivering nicotine to the lungs. Aerosol droplets of a large particle size tend to be deposited in the mouth and/or upper respiratory tract. Aerosol particles of a small (e.g., sub-micron) particle size can be inhaled into the lungs but may be exhaled without delivering nicotine to the lungs. As a result, the user would require drawing a longer puff, more puffs, or vaporizing e-liquid with a higher nicotine concentration in order to achieve the desired experience.

Based on the insight of the present inventors, a factor associated with generating suitable aerosol droplet size distributions in typical smoking substitute systems is considered to be the velocity of air flowing over the heater which generates aerosol droplets. In some prior art systems, a single air flow passage extends between an air inlet and a mouthpiece with an aerosol generator disposed in the passage. When a user inhales at the mouthpiece, an air flow is generated in the passage that passes over the aerosol generator. As there is only one passage through which the air can flow, the air velocity over the aerosol generator is determined by the inhalation flow rate imposed by the user. As such, if the user inhales too vigorously, the air velocity over the aerosol generator becomes too large to produce aerosol droplets of a suitable size distribution, thus lowering the efficiency of nicotine delivery for the system.

Accordingly, there is a need for improvement in the delivery of nicotine to a user in the context of a smoking substitute system.

The present disclosure (Development F) has been devised in the light of the above considerations.

In a general aspect of Development F, the present disclosure provides a bypass flow passage, permitting division of an air flow between the bypass flow passage and a main flow passage, wherein a flow regulator presents a variable flow resistance to air flow in the bypass passage to vary a proportion of air flow through the bypass flow passage compared with the air flow through the main flow passage.

According to a first preferred aspect of Development F there is provided a smoking substitute apparatus comprising:
a main flow passage formed between a main air inlet and a main outlet;
at least one bypass flow passage formed between a bypass air inlet and a bypass outlet, wherein air flows in use along the main flow passage and along the bypass flow passage for inhalation by a user drawing on the apparatus;
an aerosol generator operable to generate an aerosol from an aerosol precursor, the aerosol generator being in communication with the main flow passage;
wherein there is provided at least one flow regulator for varying a proportion of air flow through the bypass flow passage compared with air flow through the main flow passage, the flow regulator presenting a variable flow resistance to air flow in the bypass flow passage, said flow resistance depending on an air pressure difference across the flow regulator in view of a rate of flow demanded through the apparatus by the user.

Advantageously, the arrangement of the flow passages allows a flow demanded by a user drawing air through the apparatus, e.g., by inhalation, to be shared between the main and bypass flow passages. Hence, when a user inhales from the apparatus, the rate of flow in the main passage, and hence the air velocity over the aerosol generator, is lower than a corresponding case where the bypass passage is absent. This is useful in cases where the user inhales with a high flow rate, as it prevents the air velocity over the aerosol generator from becoming too high to generate suitably sized aerosol droplets.

The flow regulator provides a further advantage by allowing the proportions of air flow through the bypass flow passage and the main flow passage to be varied according to an air pressure difference across the flow regulator. For example, when a user demands a higher rate of flow, the pressure difference across the flow regulator is higher, and the flow regulator reduces the resistance to air flow through the bypass passage. This decreases the proportion of air flow through the main flow passage, which decreases the velocity of air over the aerosol generator to a suitable level, allowing aerosol droplets of a suitable size distribution to be generated by the aerosol generator. Conversely, when a user demands a lower rate of flow, the pressure difference across the flow regulator is lower, and the flow regulator increases the resistance to air flow through the bypass passage. This increases the proportion of air flow through the main flow passage, which increases the velocity of air over the aerosol generator to a suitable level, again allowing aerosol droplets of a suitable size distribution to be generated by the aerosol generator.

Optionally, the main air inlet and the bypass air inlet may be separate. In other embodiments, the main outlet and the bypass outlet may be constituted by a common outlet.

Conveniently, the flow regulator may include a resiliently deformable member configured to vary the flow resistance to air flow in the bypass passage by resiliently deforming to a variable extent depending on the air pressure difference across the flow regulator. The use of a resiliently deformable member provides a simple means for varying flow resistance depending on an air pressure difference. For example, the resiliently deformable member can be configured to deform, so as to vary the cross sectional area of flow through the flow regulator, according to the air pressure thereacross. The resiliently deformable member may be an annular member attached circumferentially to an inner wall of the bypass flow passage.

In other embodiments, the flow regulator may include a member hinged with respect to a wall of the bypass flow passage, the member being configured to vary the flow resistance to air flow in the bypass passage by hinging about the wall of the bypass flow passage so as to vary the cross sectional area of flow through the regulator.

Advantageously, the smoking substitute apparatus may further include a mouthpiece having a bypass channel for engaging with the bypass passage so as to be in fluid communication therewith, and a main channel for engaging with the main passage so as to be in fluid communication therewith, wherein at least one flow regulator is situated in the bypass channel of the mouthpiece.

The aerosol generator may include a heater. The aerosol precursor may be a liquid. Thus, the aerosol generator may operate to vaporize the aerosol precursor using heat generated by the heater, the vapor subsequently forming an aerosol entrained in air flowing along the main passage.

The smoking substitute apparatus may be configured to generate an aerosol having a median droplet size, d₅₀, of at least 1 μm.

According to a second aspect of Development F of the disclosure there is provided a smoking substitute device configured to engage with the smoking substitute apparatus of the first aspect, wherein the device comprises a controller and a power source configured to energize the aerosol generator.

According to a third aspect of Development F of the disclosure there is provided a smoking substitute system for generating an aerosol, comprising: the smoking substitute apparatus of the first aspect; and the smoking substitute device of the second aspect.

According to a fourth aspect of Development F of the disclosure there is provided a method of generating an aerosol using the smoking substitute apparatus of the first aspect, wherein droplets of the aerosol have a median diameter, d₅₀, of at least 1 μm.

Development G

For a smoking substitute system, it is desirable to deliver nicotine into the user's lungs, where it can be absorbed into the bloodstream. However, the present disclosure is based in part on a realization that some prior art smoking substitute systems, such delivery of nicotine is not efficient. In some prior art systems, the aerosol droplets have a size distribution that is not suitable for delivering nicotine to the lungs. Aerosol droplets of a large particle size tend to be deposited in the mouth and/or upper respiratory tract. Aerosol particles of a small (e.g., sub-micron) particle size can be inhaled into the lungs but may be exhaled without delivering nicotine to the lungs. As a result, the user would require drawing a longer puff, more puffs, or vaporizing e-liquid with a higher nicotine concentration in order to achieve the desired experience.

Based on the insight of the present inventors, it is considered that a factor associated with generating suitable aerosol droplet size distributions in typical smoking substitute systems is the velocity of air flowing over a vaporizer (or more generally an aerosol generator) that generates aerosol droplets. In some prior art systems, a single air flow passage extends between an air inlet and a mouthpiece with a vaporizer disposed in the passage. When a user inhales at the mouthpiece, an air flow is generated in the passage that passes over the vaporizer. As there is only one passage through which the air can flow, the air velocity over the vaporizer is determined by the inhalation flow rate of the user. As such, if the user inhales too vigorously, the air velocity over the vaporizer becomes too large to generate aerosol droplets of a suitable size distribution, thus lowering the efficiency of nicotine delivery for the system.

Accordingly, there is a need for improvement in the delivery of nicotine to a user in the context of a smoking substitute system.

The present disclosure (Development G) has been devised in the light of the above considerations.

In a general aspect of Development G, the present disclosure provides a bypass flow channel, permitting division of an air flow between the bypass flow channel and a main flow channel, the bypass channel having a bypass constriction region determining the resistance to flow along the bypass flow channel. The main channel optionally has a main constriction region determining the resistance to flow along the main flow channel.

According to a first preferred aspect of Development G there is provided a smoking substitute apparatus comprising: a housing; one or more outlets formed at the housing; a main air inlet and a bypass air inlet respectively formed at the housing; wherein the one or more outlets are configured to be in fluid communication with the main air inlet and the bypass air inlet provided at the housing through a respective main flow channel and a bypass flow channel; an aerosol generator positioned along the main flow channel for generating an aerosol from an aerosol precursor; a bypass constriction region provided along the bypass flow channel, the bypass constriction presenting a smaller cross sectional area to flow than that of the bypass air inlet and that of the one or more outlets, the cross sectional area of the bypass constriction region determining the resistance to flow along the bypass flow channel and thereby the ratio of flow between the bypass flow in the bypass flow channel and the main flow in the main flow channel; and optionally, the smoking substitute apparatus further comprising a main constriction region provided along the main flow channel, the main constriction presenting a smaller cross sectional area to flow than that of the main air inlet, wherein the cross sectional area of the main constriction region determines the resistance to flow along the main flow channel, and thereby the main and bypass constriction regions determine the ratio of flow between the bypass flow in the bypass flow channel and the main flow in the main flow channel, to provide the total rate of flow at the one or more outlets.

Advantageously, the arrangement of the flow channels allows a flow demanded at the one or more outlets (typically caused by a user drawing air through the apparatus, e.g., by inhalation) to be shared between the main and bypass flow channels. Hence, when a user inhales from the apparatus, the rate of flow in the main channel, and hence the air velocity over the aerosol generator, is lower than a corresponding case where the bypass channel is absent. This is useful in cases where the user inhales with a high flow rate, as it prevents the air velocity over the aerosol generator from becoming too high to generate suitably sized aerosol droplets.

The main constriction, if present, and the bypass constriction provide a further advantage, by allowing the ratio of flow between the main and bypass flow channels to be precisely set. The constrictions achieve this by being the determining factor in the air flow resistance in both the main flow channel and the bypass flow channel, thus controlling the share of the air flow that passes through the bypass channel and the main flow channel. Additionally, as the constrictions may be present in both the main and bypass flow channels, the flow resistance of the whole apparatus, and thus the rate of flow at the one or more outlets, may be controlled. Therefore, by implementing particular main and bypass constrictions, the air velocity over the aerosol generator in the main flow channel can be tailored to an appropriate level for generation of suitably sized aerosol droplets, for users with varying inhalation flow rates, and the apparatus can restrict users to particular inhalation flow rates.

Optionally, at least one of the main constriction region, if present, and the bypass constriction region may include a tapered region of the flow channel in which it is disposed, the cross sectional area of the channel tapering to a smaller cross sectional area than that of the respective air inlet of the flow channel.

Conveniently, at least one of the main constriction region, if present, and the bypass constriction region may include a section of uniform cross sectional area, the section having a cross sectional area smaller than that of the respective air inlet of the flow channel.

Advantageously, at least one of the main constriction region, if present, and the bypass constriction region may include a mesh such as a foraminous mesh which partially blocks the flow channel in which it is disposed.

Optionally, at least one of the main constriction region, if present, and the bypass constriction region may be configured to generate turbulent air flow along the flow channel in which it is disposed. For example, at least one of the main constriction region, if present, and the bypass constriction region may include one or more walls having a surface that is rougher than that of the walls of the remainder of the flow channel in which it is disposed.

In some embodiments, it is advantageous for at least one of the main constriction region, if present, and the bypass constriction region to be adjustable to change the resistance to flow along the flow channel in which it is disposed. This advantageously allows the ratio of flow between the bypass flow and the main flow to be varied according to the inhalation rate of a user. For instance, at least one of the main constriction region, if present, and the bypass constriction region may include a valve. Alternatively, at least one of the main constriction region, if present, and the bypass constriction region may include two relatively rotatable members, rotatable between a first relative position and a second relative position, these positions differing in terms of the cumulative obstruction presented to air flow. For example, the rotatable members may be foraminous meshes. In this case, the foramina of the meshes may be aligned relative to the flow channel, in which the at least one of the main constriction region, if present, and the bypass constriction region is disposed, in the first relative position, and in the second relative position, the foramina may be misaligned relative to the flow channel in which the at least one of the main constriction region, if present, and the bypass constriction region is disposed.

Optionally, the smoking substitute apparatus may be configured to generate an aerosol having a median droplet size, d₅₀, of at least 1 μm.

The aerosol generator may include a heater. The aerosol precursor may be a liquid. Thus, the aerosol generator may operate to vaporize the aerosol precursor using heat generated by the heater, the vapor subsequently forming an aerosol entrained in air flowing along the main channel.

According to a second aspect of Development G of the disclosure there is provided a smoking substitute device configured to engage with the smoking substitute apparatus of the first aspect, wherein the device comprises a controller and a power source configured to energize the aerosol generator.

According to a third aspect of Development G of the disclosure, there is provided a smoking substitute system for generating an aerosol, comprising:
the smoking substitute apparatus of the first aspect; and
the smoking substitute device of the second aspect.

According to a fourth aspect of Development G of the disclosure, there is provided a method of generating an aerosol using the smoking substitute apparatus of the first aspect, wherein droplets of the aerosol have a median diameter, d₅₀, of at least 1 μm.

Development H

For a smoking substitute system, it is desirable to deliver nicotine into the user's lungs, where it can be absorbed into the bloodstream. However, the present disclosure is based in part on a realization that some prior art smoking substitute systems, such delivery of nicotine is not efficient. In some prior art systems, the aerosol droplets have a size distribution that is not suitable for delivering nicotine to the lungs. Aerosol droplets of a large particle size tend to be deposited in the mouth and/or upper respiratory tract. Aerosol particles of a small (e.g., sub-micron) particle size can be inhaled into the lungs but may be exhaled without delivering nicotine to the lungs. As a result, the user would require drawing a longer puff, more puffs, or vaporizing e-liquid with a higher nicotine concentration in order to achieve the desired experience.

Accordingly, there is a need for improvement in the delivery of nicotine to a user in the context of a smoking substitute system.

The present disclosure (Development H) has been devised in the light of the above considerations.

In a general aspect of Development H, the present disclosure relates to a smoking substitute apparatus with a main passage and a bypass passage, comprising a means for constricting the air flow through the bypass passage.

According to a first preferred aspect of Development H there is provided a smoking substitute apparatus comprising one or more air inlets, one or more outlets arranged at a mouthpiece, a first passage leading from at least one of the air inlets to at least one of the outlets at the mouthpiece, an aerosol generator arranged in a vaporization chamber in the first passage, the aerosol generator being operable to generate an aerosol from an aerosol precursor to flow in use along the first passage downstream of the aerosol generator for inhalation by a user drawing on the mouthpiece, and a second passage leading from at least one of the air inlets to at least one of the outlets at the mouthpiece, wherein the second passage bypasses the vaporization chamber of the first passage, and further wherein the mouthpiece comprises a flow constrictor for constricting the flow of air in the second passage.

Providing a flow constrictor in the mouthpiece may allow the relative rates of airflow in the first and second passage to be controlled so as to optimize flow rates for larger droplet formation.

Optionally, the mouthpiece may be a releasably engageable part of the smoking substitute apparatus.

Providing a releasably engageable mouthpiece may allow a user to change the mouthpiece for one that provides a different (i.e., higher or lower) level of constriction, as required by their typical draw rate.

Conveniently, the flow constrictor may be a releasably engageable component of the mouthpiece.

Providing a releasably engageable flow constrictor may allow a user to change the flow constrictor for one that provides a different (i.e., higher or lower) level of constriction, as required by their typical draw rate, while minimizing the number of parts requiring replacement.

Advantageously, the aerosol generator may comprise a heater operable to generate the aerosol from the aerosol precursor.

Optionally, the aerosol generator may comprise a porous wick which, in use, wicks aerosol precursor from a reservoir to the first passage for entrainment in air flowing downstream of the aerosol generator.

Conveniently, the heater may comprise a heating filament that is wound around a portion of the porous wick.

Advantageously, the vaporization chamber may have a larger cross sectional diameter than a downstream part of the first passage.

Optionally, the part of the first passage bypassed by the second passage may comprise a flow conditioning apparatus arranged upstream of the aerosol generator which, when the smoking substitute apparatus is in use, reduces turbulence in flow at the aerosol generator.

Conveniently, the flow conditioning apparatus may comprise a mesh arranged in the first passage such that, in use, the flow generated by a user drawing on the mouthpiece passes through the mesh.

Advantageously, the first passage, the second passage and flow constrictor can be configured such that, in use, the flow rate in the first passage is more than 1/20 of the flow rate in the second passage.

Optionally, the first passage, the second passage and flow constrictor can be configured such that, in use, the flow rate in the first passage is less than twice of the flow rate in the second passage.

Conveniently, in use, the d₅₀ particle size of the aerosol particles generated by the aerosol generator may be greater than 1 μm.

Advantageously, in use, the d₅₀ particle size of the aerosol particles generated by the aerosol generator may be less than 10 μm.

Optionally, in use, the span of particle size distribution, defined as (d₉₀−d₁₀)/d₅₀, may be less than 20.

Conveniently, the smoking substitute apparatus may be comprised by or within a cartridge configured for engagement with a base unit, the cartridge and base unit together forming a smoking substitute system.

In another aspect of Development H, a smoking substitute system is provided, the smoking substitute system comprising a base unit, and a smoking substitute apparatus comprised by or within a cartridge, wherein the smoking substitute apparatus is removably engageable with the base unit.

In a third aspect of Development H, a mouthpiece is provided that is configured for engagement with the smoking substitute apparatus.

In a fourth aspect of Development H, a flow constrictor is provided that is configured for engagement with the mouthpiece of the smoking substitute apparatus.

In a fifth aspect of Development H, a kit of parts for a smoking substitute apparatus is provided, the kit comprising a smoking substitute apparatus and a plurality of mouthpieces, wherein each of the plurality of mouthpieces comprises a flow constrictor which is configured to constrict the flow of air in the second passage by a respectively different level of constriction.

In a sixth aspect of Development H, a kit of parts for a smoking substitute apparatus is provided, the kit comprising a smoking substitute apparatus and a plurality of flow constrictors, wherein each of the plurality of flow constrictors is configured to constrict the flow of air in the second passage by a respectively different level of constriction.

In a seventh aspect of Development H, a method of using a smoking substitute apparatus of the first aspect of Development H, or of using a kit according to the fifth or sixths aspects of Development H, is provided.

Optionally, the method of using the smoking substitute apparatus may comprise the step of selecting a flow constrictor providing a desired level of constriction from a plurality of flow constrictors providing different levels of constriction.

Development I

For a smoking substitute system, it is desirable to deliver nicotine into the user's lungs, where it can be absorbed into the bloodstream. However, the present disclosure is based in part on a realization that some prior art smoking substitute systems, such delivery of nicotine is not efficient. In some prior art systems, the aerosol droplets have a size distribution that is not suitable for delivering nicotine to the lungs. Aerosol droplets of a large particle size tend to be deposited in the mouth and/or upper respiratory tract. Aerosol particles of a small (e.g., sub-micron) particle size can be inhaled into the lungs but may be exhaled without delivering nicotine to the lungs. As a result, the user would require drawing a longer puff, more puffs, or vaporizing e-liquid with a higher nicotine concentration in order to achieve the desired experience.

Accordingly, there is a need for improvement in the delivery of nicotine to a user in the context of a smoking substitute system.

The present disclosure (Development I) has been devised in the light of the above considerations.

In a general aspect of Development I, embodiments of the present disclosure relate to a smoking substitute apparatus including one or more adjustable airflow restrictors.

According to a first preferred aspect of Development I there is provided a smoking substitute apparatus comprising:
a vaporizer chamber, including a vaporizer configured to vaporize a vaporizable liquid;
a primary airflow path, which passes from a first air inlet of the smoking substitute apparatus through the vaporizer chamber, to an outlet of the smoking substitute apparatus;
a secondary airflow path, which passes from a second air inlet of the smoking substitute apparatus to the outlet of the smoking substitute apparatus, said secondary airflow path bypassing the vaporizer chamber and passing through one or more bypass air ducts;
wherein one or more adjustable airflow restrictors are disposed along either or both of the primary airflow path and the secondary airflow path, said adjustable airflow restrictors being adjustable by a user of the device to vary a draw resistance of the smoking substitute apparatus.

By providing such adjustable airflow restrictors, the inhalation profile (e.g., draw resistance, and other characteristics) can be adjusted to suit the user whilst not distorting the airflow through the vaporizer chamber. Accordingly, advantageous airflow profiles over the vaporizer chamber can be preserved. Moreover, a split or division or airflow between the primary and secondary airflow paths can be modified to cause a preferential airflow through one or other of the primary and secondary airflow paths. For example, if the adjustable airflow restrictor is located along the primary airflow path, air may preferentially flow along the secondary airflow path.

Optionally, the or each airflow restrictor may be a constriction along the respective airflow path, the constriction having an adjustable cross-section. In some examples, the constriction may have an adjustable width.

Advantageously, the or each airflow restrictor may be adjustable through the application of a force, by the user, to a portion of an outer housing of the smoking substitute apparatus.

Conveniently, the or each airflow restrictor may be adjustable through adjustment of a dial attached to a portion of an outer housing of the smoking substitute apparatus.

Optionally, the or each airflow restrictor, which may be disposed along the secondary airflow path, may be located within each of the bypass air ducts.

Conveniently, the or each airflow restrictor, which may be disposed along the primary airflow path, may be located between the first air inlet and a vaporizer chamber inlet of the vaporizer chamber.

Additionally, the smoking substitute apparatus may include two first air inlets, located on respectively opposite sides of the smoking substitute apparatus.

In some embodiments, the smoking substitute apparatus may include two second air inlets, located on respectively opposite sides of the smoking substitute apparatus.

Optionally, the first inlet may provide air to both the primary and secondary airflow path.

Conveniently, the primary airflow path and the secondary airflow path may converge in the outlet of the smoking substitute apparatus. The outlet may be a mouthpiece of the smoking substitute apparatus.

In some embodiments, the primary airflow path and the secondary airflow path may partially overlap, and the or each airflow restrictor may be disposed along the overlapping portion of the airflow paths.

Additionally, the vaporizer chamber may include a flow straightener located between a vaporizer chamber inlet and the vaporizer, which may be configured to induce a laminar airflow over the vaporizer.

Optionally, the vaporizer chamber may include a plenum, located between a vaporizer chamber inlet and the vaporizer, which may be configured to reduce an airflow velocity over the vaporizer.

In some embodiments, the smoking substitute apparatus may include a vaporizer chamber outlet, located between the vaporizer and the outlet of the smoking substitute apparatus, and the vaporizer chamber outlet may be a tapered chimney.

In a second aspect of Development I, embodiments of the disclosure provide a smoking substitute system, including the smoking substitute apparatus of the first aspect (and including any, or any combination insofar as they are compatible, of the optional features disclosed therein) and a main body, the main body including a power source for the vaporizer.

The disclosure includes the combination of the developments, aspects and preferred features described except where such a combination is clearly impermissible or expressly avoided.

### BRIEF DESCRIPTION OF THE DRAWINGS

So that the disclosure may be understood, and so that further developments, aspects and features thereof may be appreciated, embodiments illustrating the principles of the disclosure will now be discussed in further detail with reference to the accompanying figures, in which:

FIG. 1 illustrates a set of rectangular tubes for use in experiments to assess the effect of flow and cooling conditions at the wick on aerosol properties. Each tube has the same depth and length but different width.

FIG. 2 shows a schematic perspective longitudinal cross sectional view of an example rectangular tube with a wick and heater coil installed.

FIG. 3 shows a schematic transverse cross sectional view an example rectangular tube with a wick and heater coil installed. In this example, the internal width of the tube is 12 mm.

FIGS. 4A-4D show air flow streamlines in the four devices used in a turbulence study.

FIG. 5 shows the experimental set up to investigate the influence of inflow air temperature on aerosol particle size, in order to investigate the effect of vapor cooling rate on aerosol generation.

FIG. 6 shows a schematic longitudinal cross sectional view of a first smoking substitute apparatus (pod 1) used to assess influence of inflow air temperature on aerosol particle size.

FIG. 7 shows a schematic longitudinal cross sectional view of a second smoking substitute apparatus (pod 2) used to assess influence of inflow air temperature on aerosol particle size.

FIG. 8A shows a schematic longitudinal cross sectional view of a third smoking substitute apparatus (pod 3) used to assess influence of inflow air temperature on aerosol particle size.

FIG. 8B shows a schematic longitudinal cross sectional view of the same third smoking substitute apparatus (pod 3) in a direction orthogonal to the view taken in FIG. 8A.

FIG. 9 shows a plot of aerosol particle size (Dv50) experimental results against calculated air velocity.

FIG. 10 shows a plot of aerosol particle size (Dv50) experimental results against the flow rate through the apparatus for a calculated air velocity of 1 m/s.

FIG. 11 shows a plot of aerosol particle size (Dv50) experimental results against the average magnitude of the velocity in the vaporizer surface region, as obtained from CFD modelling.

FIG. 12 shows a plot of aerosol particle size (Dv50) experimental results against the maximum magnitude of the velocity in the vaporizer surface region, as obtained from CFD modelling.

FIG. 13 shows a plot of aerosol particle size (Dv50) experimental results against the turbulence intensity.

FIG. 14 shows a plot of aerosol particle size (Dv50) experimental results dependent on the temperature of the air and the heating state of the apparatus.

FIG. 15 shows a plot of aerosol particle size (Dv50) experimental results against vapor cooling rate to 50° C.

FIG. 16 shows a plot of aerosol particle size (Dv50) experimental results against vapor cooling rate to 75° C.

FIG. 17 is a schematic front view of a smoking substitute system, according to a first embodiment, in an engaged position;

FIG. 18 is a schematic front view of the smoking substitute system of the first embodiment in a disengaged position;

FIG. 19 is a schematic longitudinal cross sectional view of a smoking substitute apparatus of a first reference arrangement;

FIG. 20 is an enlarged schematic cross sectional view of part of the air passage and vaporization chamber of a first reference arrangement;

FIG. 21 is a schematic longitudinal cross sectional view of a smoking substitute apparatus of the first embodiment of Development A;

FIG. 22 is an enlarged schematic cross sectional view of part of the air passage of the first embodiment of Development A;

FIG. 23 is an enlarged schematic top-down view of a flow conditioning apparatus of the first embodiment of Development A;

FIG. 24 is a schematic longitudinal cross sectional view of a smoking substitute apparatus of a second reference arrangement of Development A;

FIG. 25 is a schematic longitudinal cross sectional view of a smoking substitute apparatus of a third reference arrangement of Development A; and

FIG. 26 is a schematic longitudinal cross sectional view of a smoking substitute apparatus of a fourth reference arrangement of Development A.

FIG. 27 is a schematic view of a heat shield arrangement of an embodiment of Development B;

FIG. 28 is a schematic plan view projection of a heater and a wick onto a heat-absorbing surface of a heat shield plate.

FIG. 29 is a schematic cross sectional view of a further heat shield arrangement of an embodiment of Development B;

FIG. 30 is a second view of the further heat shield arrangement of FIG. 29;

FIG. 31 shows a schematic cross sectional view of a smoking substitute apparatus of a second reference arrangement of Development B; and

FIG. 32 shows a schematic cross sectional view of a smoking substitute apparatus of a third reference arrangement of Development B.

FIG. 33 is a schematic longitudinal cross sectional view of a smoking substitute apparatus of the first embodiment of Development C.

FIG. 34 is a schematic longitudinal cross sectional view of a smoking substitute apparatus of a first embodiment of Development D.

FIG. 35 is a schematic longitudinal cross sectional view of a smoking substitute apparatus of a second embodiment of Development D;

FIG. 36 is a schematic longitudinal cross sectional view of a smoking substitute apparatus of a third embodiment of Development D;

FIG. 37 is a schematic longitudinal cross sectional view of a smoking substitute apparatus of a fourth embodiment of Development D;

FIG. 38 is an enlarged schematic cross sectional view of part of the air passage of the smoking substitute apparatus according to Development D; and

FIG. 39 is an enlarged schematic top-down view of a flow conditioning apparatus of the smoking substitute apparatus according to of Development D.

FIG. 40 is a schematic longitudinal cross sectional view of a smoking substitute apparatus of a first embodiment of Development E;

FIG. 41 is a top-down cross-sectional view of a smoking substitute apparatus of a second embodiment of Development E;

FIG. 42 is an enlarged schematic cross sectional view of part of the air passage of a smoking substitute apparatus of third embodiment of Development E;

FIG. 43 is an enlarged schematic cross sectional view of part of the air passage of the smoking substitute apparatus according to Development E; and

FIG. 44 is an enlarged schematic top-down view of a flow conditioning apparatus of the smoking substitute apparatus according to Development E.

FIG. 45 is a schematic longitudinal cross sectional view of a smoking substitute apparatus of the first embodiment of Development F;

FIGS. 46A, 46B, and 46C provide a set of schematic views of an annular resiliently deformable member, disposed in a bypass flow passage, acting as a flow regulator;

FIG. 47 is a schematic view of a hinged member disposed in a bypass flow passage acting as a flow regulator;

FIG. 48 is a schematic longitudinal cross sectional view of a smoking substitute apparatus of a second embodiment of Development F; and

FIG. 49 is an enlarged schematic longitudinal cross sectional view of a mouthpiece of the second embodiment of Development F.

FIG. 50 is a schematic longitudinal cross sectional view of a smoking substitute apparatus of a first embodiment of Development G.

FIG. 51 is a schematic longitudinal cross sectional view of a smoking substitute apparatus of a second embodiment of Development G.

FIG. 52 is a schematic longitudinal cross sectional view of a smoking substitute apparatus of the first embodiment of Development H;

FIG. 53 is a schematic longitudinal cross sectional view of a smoking substitute apparatus of the second embodiment of Development H with a mouthpiece in an engaged position;

FIG. 54 is a schematic longitudinal cross sectional view of a mouthpiece of a smoking substitute apparatus of the second embodiment of Development H;

FIG. 55 is a schematic longitudinal cross sectional view of a smoking substitute apparatus of the second embodiment of Development H with the mouthpiece removed; and

FIG. 56 is a schematic longitudinal cross sectional view of a mouthpiece of a smoking substitute apparatus of the third embodiment of Development H.

FIG. 57 is an enlarged schematic cross sectional view of part of the air passage and vaporization chamber of an embodiment of Development H incorporating a flow conditioning apparatus.

FIG. 58 shows a schematic plan view of a flow conditioning apparatus for use in an embodiment of Development H.

FIG. 59 is a schematic longitudinal cross sectional view of a smoking substitute apparatus of the first embodiment of Development I;

FIG. 60 is a schematic longitudinal cross sectional view of a smoking substitute apparatus of a second embodiment of Development I; and

FIG. 61 is a schematic longitudinal cross sectional view of a smoking substitute apparatus of a third embodiment of Development I.

### DETAILED DESCRIPTION

Further background to the present disclosure and further aspects and embodiments of the present disclosure will now be discussed with reference to the accompanying figures. Further aspects and embodiments will be apparent to those skilled in the art. The contents of all documents mentioned in this text are incorporated herein by reference in their entirety.

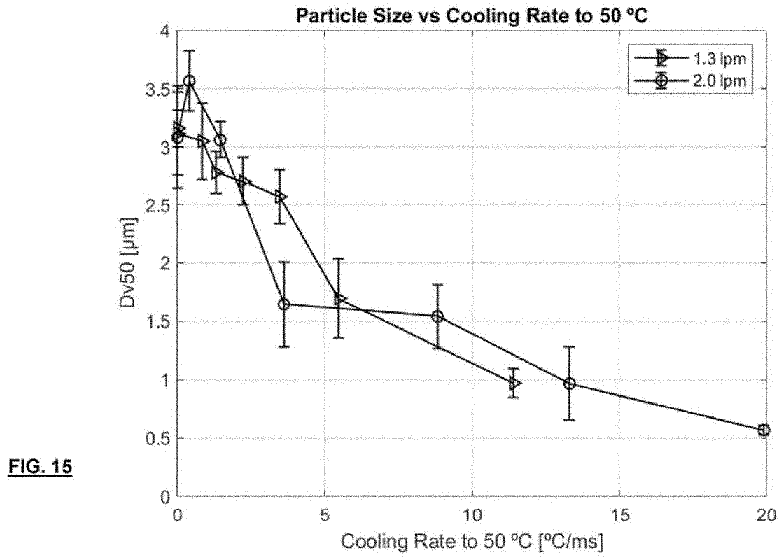

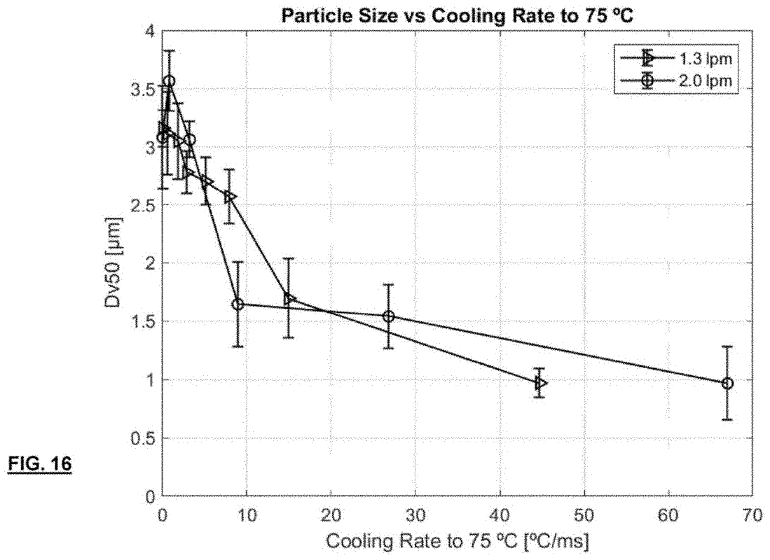

FIGS. 17 and 18 illustrate a smoking substitute system in the form of an e-cigarette system 110. The system 110 comprises a main body 120 of the system 110, and a smoking substitute apparatus in the form of an e-cigarette consumable (or “pod”) 150. In the illustrated embodiment the consumable 150 (sometimes referred to herein as a smoking substitute apparatus) is removable from the main body 120, so as to be a replaceable component of the system 110. The e-cigarette system 110 is a closed system in the sense that it is not intended that the consumable should be refillable with e-liquid by a user.

As is apparent from FIGS. 17 and 18, the consumable 150 is configured to engage the main body 120. FIG. 17 shows the main body 120 and the consumable 150 in an engaged state, whilst FIG. 18 shows the main body 120 and the consumable 150 in a disengaged state. When engaged, a portion of the consumable 150 is received in a cavity of corresponding shape in the main body 120 and is retained in the engaged position by way of a snap-engagement mechanism. In other embodiments, the main body 120 and consumable 150 may be engaged by screwing one into (or onto) the other, or through a bayonet fitting, or by way of an interference fit.

The system 110 is configured to vaporize an aerosol precursor, which in the illustrated embodiment is in the form of a nicotine-based e-liquid 160. The e-liquid 160 comprises nicotine and a base liquid including propylene glycol and/or vegetable glycerin. In the present embodiment, the e-liquid 160 is flavored by a flavorant. In other embodiments, the e-liquid 160 may be flavorless and thus may not include any added flavorant.

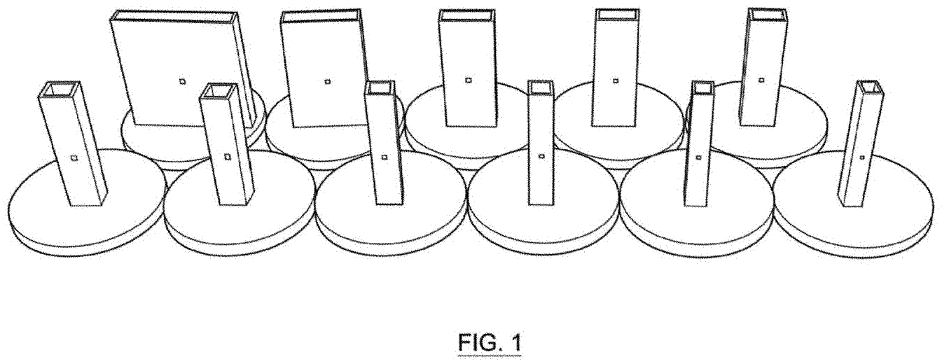

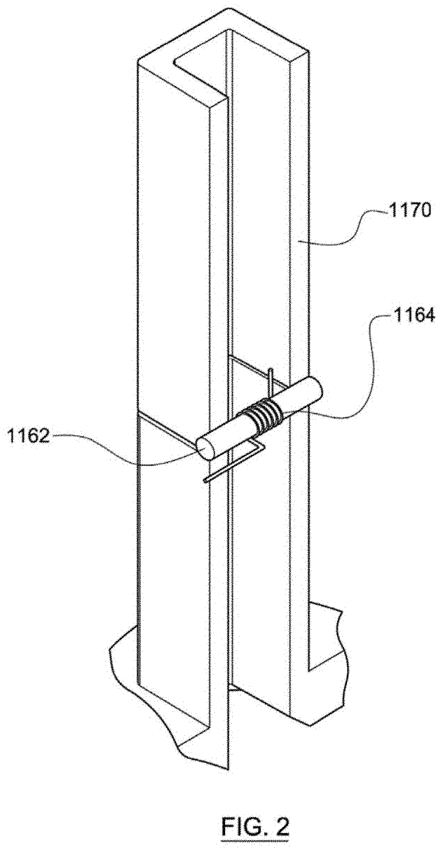

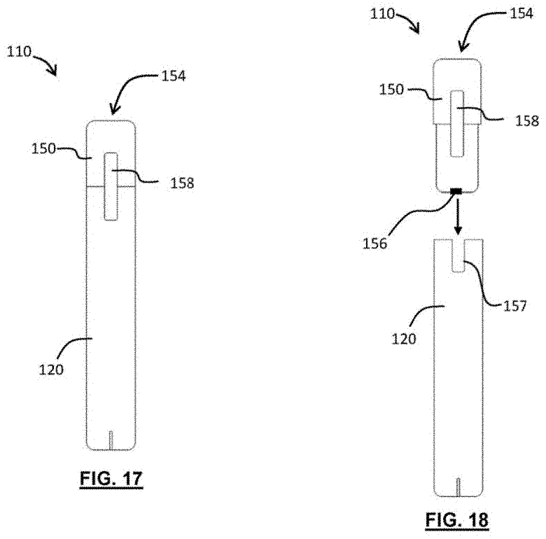

FIG. 19 shows a schematic longitudinal cross sectional view of a first reference arrangement of the smoking substitute apparatus forming part of the smoking substitute system shown in FIGS. 17 and 18. In FIG. 19, the e-liquid 160 is stored within a reservoir in the form of a tank 152 that forms part of the consumable 150. In the illustrated first reference arrangement, the consumable 150 is a “single-use” consumable 150. That is, upon exhausting the e-liquid 160 in the tank 152, the intention is that the user disposes of the entire consumable 150. The term “single-use” does not necessarily mean the consumable is designed to be disposed of after a single smoking session. Rather, it defines the consumable 150 is not arranged to be refilled after the e-liquid contained in the tank 152 is depleted. The tank may include a vent (not shown) to allow ingress of air to replace e-liquid that has been used from the tank. The consumable 150 advantageously includes a window 158 (see FIGS. 1 and 2), so that the amount of e-liquid in the tank 152 can be visually assessed. The main body 120 includes a slot 157 so that the window 158 of the consumable 150 can be seen whilst the rest of the tank 152 is obscured from view when the consumable 150 is received in the cavity of the main body 120. The consumable 150 may be referred to as a “clearomizer” when it includes a window 158, or a “cartomizer” when it does not.

In some embodiments, the e-liquid (i.e., aerosol precursor) may be the only part of the system that is truly “single-use”. That is, the tank may be refillable with e-liquid or the e-liquid may be stored in a non-consumable component of the system. For example, in such other embodiments, the e-liquid may be stored in a tank located in the main body or stored in another component that is itself not single-use (e.g., a refillable cartomizer).

The external wall of tank 152 is provided by a casing of the consumable 150. The tank 152 annularly surrounds, and thus defines a portion of, a passage 170 that extends between a vaporizer inlet 172 and an outlet 174 at opposing ends of the consumable 150. In this respect, the passage 170 comprises an upstream end at the end of the consumable 150 that engages with the main body 120, and a downstream end at an opposing end of the consumable 150 that comprises a mouthpiece 154 of the system 110. The passage 170 may also be referred to as a vaporizer passage. The vaporizer passage 170 corresponds to the first air passage of the claims.

When the consumable 150 is received in the cavity of the main body 120 as shown in FIG. 17, a plurality of device air inlets 176 are formed at the boundary between the casing of the consumable and the casing of the main body. The device air inlets 176 are in fluid communication with the vaporizer inlet 172 through an inlet flow channel 178 formed in the cavity of the main body which is of corresponding shape to receive a part of the consumable 150. Air from outside of the system 110 can therefore be drawn into the passage 170 through the device air inlets 176 and the inlet flow channels 178.

When the consumable 150 is engaged with the main body 120, a user can inhale (i.e., take a puff) via the mouthpiece 154 so as to draw air through the passage 170, and so as to form an airflow (indicated by the dashed arrows in FIG. 19) in a direction from the vaporizer inlet 172 to the outlet 174. Although not illustrated, the passage 170 may be partially defined by a tube (e.g., a metal tube) extending through the consumable 150. In FIG. 19, for simplicity, the passage 170 is shown with a substantially circular cross-sectional profile with a constant diameter along its length. In some embodiments, the passage 170 may have other cross-sectional profiles, such as oval shaped or polygonal shaped profiles. Further, in some embodiments, the cross sectional profile and the diameter (or hydraulic diameter) of the passage 170 may vary along its longitudinal axis.

The smoking substitute system 110 is configured to vaporize the e-liquid 160 for inhalation by a user. To provide this operability, the consumable 150 comprises a heater having a porous wick 162 and a resistive heating element in the form of a heating filament 164 that is helically wound (in the form of a coil) around a portion of the porous wick 162. The porous wick 162 extends across the passage 170 (i.e., transverse to a longitudinal axis of the passage 170 and thus also transverse to the air flow along the passage 170 during use) and opposing ends of the wick 162 extend into the tank 152 (so as to be immersed in the e-liquid 160). In this way, e-liquid 160 contained in the tank 152 is conveyed from the opposing ends of the porous wick 162 to a central portion of the porous wick 162 so as to be exposed to the airflow in the passage 170.

The helical filament 164 is wound about the exposed central portion of the porous wick 162 and is electrically connected to an electrical interface in the form of electrical contacts 156 mounted at the end of the consumable that is proximate the main body 120 (when the consumable and the main body are engaged). When the consumable 150 is engaged with the main body 120, electrical contacts 156 make contact with corresponding electrical contacts (not shown) of the main body 120. The main body electrical contacts are electrically connectable to a power source (not shown) of the main body 120, such that (in the engaged position) the filament 164 is electrically connectable to the power source. In this way, power can be supplied by the main body 120 to the filament 164 in order to heat the filament 164. This heats the porous wick 162 which causes e-liquid 160 conveyed by the porous wick 162 to vaporize and thus to be released from the porous wick 162. The vaporized e-liquid becomes entrained in the airflow and, as it cools in the airflow (between the heated wick and the outlet 174 of the passage 170), condenses to form an aerosol. This aerosol is then inhaled, via the mouthpiece 154, by a user of the system 110. As e-liquid is lost from the heated portion of the wick, further e-liquid is drawn along the wick from the tank to replace the e-liquid lost from the heated portion of the wick.

The filament 164 and the exposed central portion of the porous wick 162 are positioned across the passage 170. More specifically, the part of passage that contains the filament 164 and the exposed portion of the porous wick 162 forms a vaporization chamber. In the illustrated reference arrangement, the vaporization chamber has the same cross-sectional diameter as the passage 170. However, in some embodiments the vaporization chamber may have a different cross sectional profile compared with the passage 170. For example, the vaporization chamber may have a larger cross sectional diameter than at least some of the downstream part of the passage 170 so as to enable a longer residence time for the air inside the vaporization chamber.

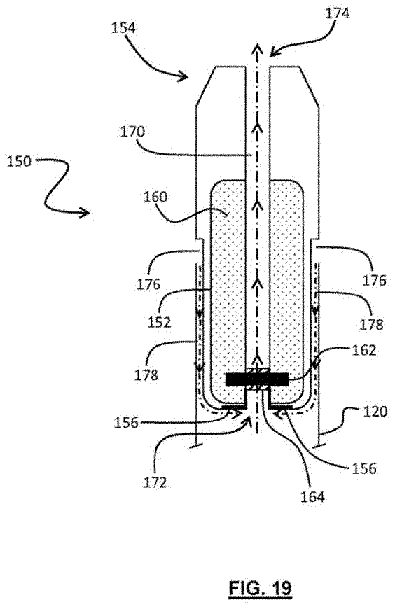

FIG. 20 illustrates in more detail the vaporization chamber and therefore the region of the consumable 150 around the wick 162 and filament 164. The helical filament 164 is wound around a central portion of the porous wick 162. The porous wick extends across passage 170. E-liquid 160 contained within the tank 152 is conveyed as illustrated schematically by arrows 401, i.e. from the tank and towards the central portion of the porous wick 162.

When the user inhales, air is drawn from through the inlets 176 shown in FIG. 19, along inlet flow channel 178 to vaporization chamber inlet 172 and into the vaporization chamber containing porous wick 162. The porous wick 162 extends substantially transverse to the airflow direction. The airflow passes around the porous wick, at least a portion of the airflow substantially following the surface of the porous wick 162. In examples where the porous wick has a cylindrical cross-sectional profile, the airflow may follow a curved path around an outer periphery of the porous wick 162.

At substantially the same time as the airflow passes around the porous wick 162, the filament 164 is heated so as to vaporize the e-liquid which has been wicked into the porous wick. The airflow passing around the porous wick 162 picks up this vaporized e-liquid, and the vapor-containing airflow is drawn in direction 403 further down passage 170.

The power source of the main body 120 may be in the form of a battery (e.g., a rechargeable battery such as a lithium-ion battery). The main body 120 may comprise a connector in the form of, e.g., a USB port for recharging this battery. The main body 120 may also comprise a controller that controls the supply of power from the power source to the main body electrical contacts (and thus to the filament 164). That is, the controller may be configured to control a voltage applied across the main body electrical contacts, and thus the voltage applied across the filament 164. In this way, the filament 164 may only be heated under certain conditions (e.g., during a puff and/or only when the system is in an active state). In this respect, the main body 120 may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 120 and consumable 150 may comprise a further interface which may, for example, be in the form of an RFID reader, a barcode or QR code reader. This interface may be able to identify a characteristic (e.g., a type) of a consumable 150 engaged with the main body 120. In this respect, the consumable 150 may include any one or more of an RFID chip, a barcode or QR code, or memory within which is an identifier and which can be interrogated via the interface.

Development A

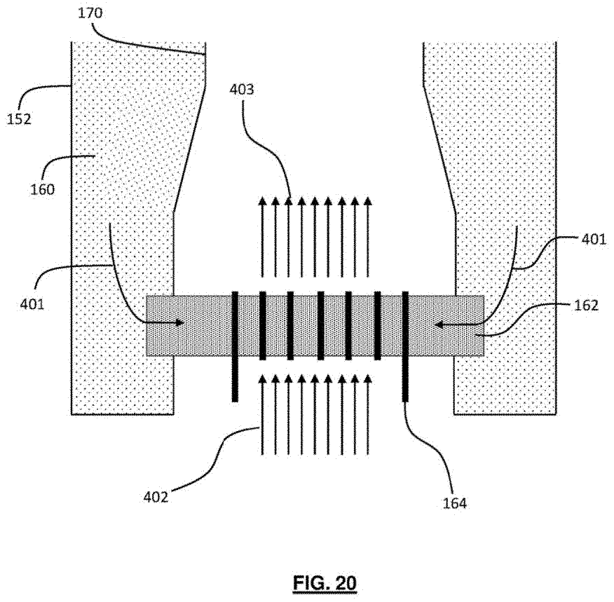

FIG. 21 illustrates a schematic longitudinal cross sectional view of a first embodiment of the smoking substitute apparatus forming part of the smoking substitute system shown in FIGS. 17 and 18. The embodiment illustrated in FIG. 21 differs from the reference arrangement illustrated in FIG. 19 in that the substitute smoking apparatus includes two bypass passages a180 in addition to the vaporizer passage a170. The bypass passages correspond to the second air passage of the claims. The bypass air passages extend between the plurality of device air inlets a176 and two outlets a184. In other embodiments, the number of bypass passages a180 and corresponding outlets a184 may be greater or smaller than in the illustrated example.

In FIG. 21, for simplicity, the bypass passage a180 is shown with a substantially circular cross-sectional profile with a constant diameter along its length. In some embodiments, the bypass passage a180 may have other cross-sectional profiles, such as oval shaped or polygonal shaped profiles. Further, in some embodiments, the cross sectional profile and the diameter (or hydraulic diameter) of the bypass passage a180 may vary along its longitudinal axis.

The provision of a bypass passage a180 means that a part of the air drawn through the smoking substitute apparatus a150a when a user inhales via the mouthpiece a154 is not drawn through the vaporization chamber. This has the effect of reducing the flow rate through the vaporization chamber in correspondence with the respective flow resistances presented by the vaporizer passage a170 and the bypass passage a180. This can reduce the correlation between the flow rate through the smoking substitute apparatus a150 (i.e., the user's draw rate) and the particle size generated when the e-liquid a160 is vaporized and subsequently forms an aerosol. Therefore, the smoking substitute apparatus a150 of the present embodiment can deliver a more consistent aerosol to a user.

Furthermore, the smoking substitute apparatus a150 of the present embodiment is capable of producing an increased particle droplet sizes, d₅₀, based on typical inhalation rates undertaken by a user, compared to the reference arrangement of FIG. 19. Such larger droplet sizes may be beneficial for the delivery of vapor to a user's lungs. The preferred ratio between the dimensions of the bypass passage a180 and the dimensions of the vaporizer passage a170, and hence flow rate in the respective passages may be determined from representative user inhalation rates and from the required air flow rate through the vaporization chamber to deliver a desired droplet size. For example, an average total flow rate of 1.3 liters per minute may be split such that 0.8 liters per minute passes through the bypass air channel a180, and 0.5 liters per minute passes through the vaporizer channel a170, a bypass:vaporizer flow rate ratio of 1.6:1. Such a flow rate may provide a median droplet size, d₅₀, of 1-3 μm (more preferably 2-3 μm) with a span of not more than 20 (preferably not more than 10). Alternative flow rate ratios may be provided based on calculations and measurements of user flow rate, vaporizer flow rate, and average droplet size d₅₀. A bypass:vaporizer flow rate ratio of between 0.5:1 and 20:1, typically at an average total flow rate of 1.3 liters per minute may be advantageous depending on the configuration of the smoking substitute apparatus.

The bypass passage and vaporizer passage extend from a common device inlet a176. This has the benefit of ensuring more consistent airflow through the bypass passage a180 and vaporizer passage a170 across the lifetime of the smoking substitute apparatus a150, since any obstruction that impinges on an air inlet a176 will affect the airflow through both passages equally. The impact of inlet manufacturing variations can also be reduced for the same reason. This can therefore improve the user experience for the smoking substitute apparatus a150. Furthermore, the provision of a common device inlet a176 simplifies the construction and external appearance of the device.

The bypass passage a180 and vaporizer passage a170 separate upstream of the vaporization chamber. Therefore, no vapor is drawn through the bypass passage a180. Furthermore, because the bypass passage leads to outlet a184 that is separate from outlet a174 of the vaporizer passage, substantially no mixing of the bypass air and vaporizer air occurs within the smoking substitute apparatus a150. Such mixing could otherwise lead to excessive cooling of the vapor and hence a build-up of condensation within the smoking substitute apparatus a150. Such condensation could have adverse implications for delivering vapor to the user, for example by causing the user to draw liquid droplets rather than vapor when “puffing” on the mouthpiece a154.

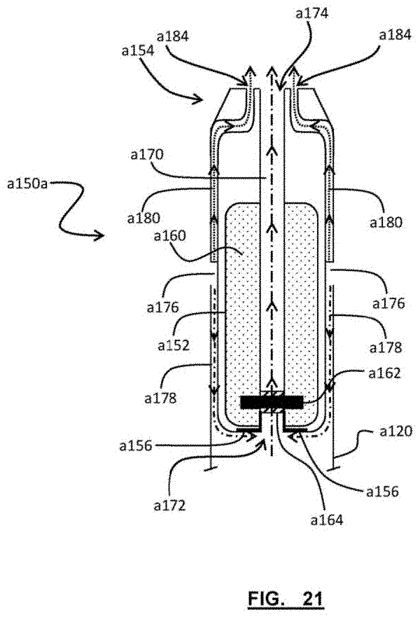

The smoking substitute apparatus may further comprise a flow conditioning apparatus a200 arranged upstream of the wick a162 and filament a164 in the vaporizer passage a170, as illustrated in FIG. 22. The flow conditioning apparatus a200 is configured to reduce turbulence in the air flow through vaporizer passage a170 and past the wick a162 and filament a164. This may be advantageous for improving aerosol generation, and for reducing the spread in the aerosol particle size distribution. In FIG. 22, the turbulent air flow is indicated by the arrows a400. Once the airflow has passed through the flow conditioning apparatus a200, the turbulence is reduced, as indicated by the aligned arrows a402.

Where the flow conditioning apparatus a200 is provided in the smoking substitute apparatus a150a, the provision of a bypass passage a180 may reduce the effect of the flow conditioning apparatus a200 on the overall resistance to draw of the smoking substitute apparatus a150a, which may in turn increase the range of possible design parameters for the flow conditioning apparatus a200 allowing air flow past the wick a162 and filament a164 to be more readily optimized for improved aerosol generation without compromising the overall operation of the smoking substitute apparatus a150a.

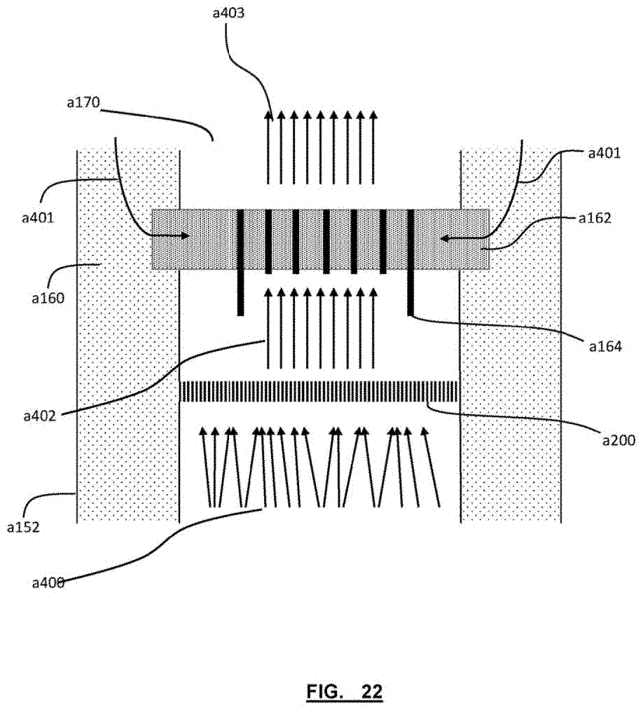

The flow conditioning apparatus a200 may, for example, comprise an air permeable mesh arranged across the vaporizer passage a170 such that air drawn through the vaporizer passage a170 is drawn through the mesh. The mesh may be constructed from an air-impermeable material comprising one or more bores a202 extending through, as illustrated exemplarily in FIG. 23.

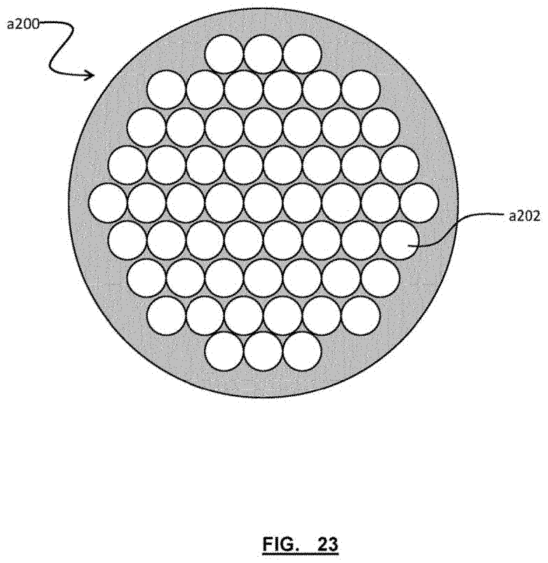

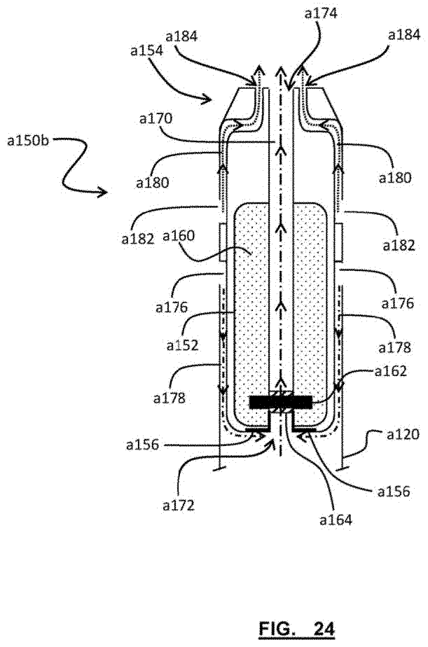

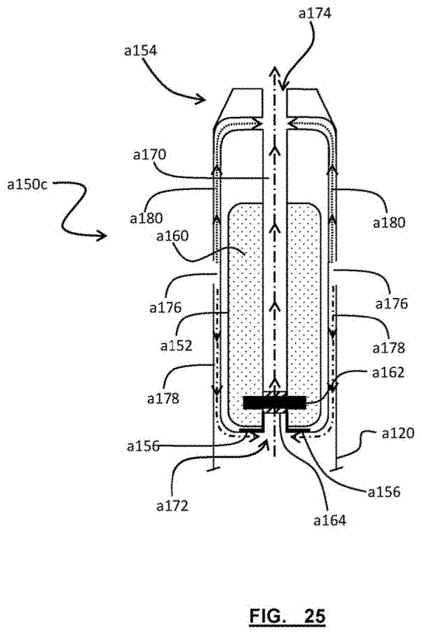

To further aid understanding of the advantages associated with the illustrated embodiment, the following second, third and fourth reference arrangements are described for purposes of comparison. Features which are common to the first embodiment illustrated in FIG. 21 will not be further explained. The second reference arrangement is as illustrated in FIG. 24. The third reference arrangement is as illustrated in FIG. 25. The fourth reference arrangement is as illustrated in FIG. 26.

In the second reference arrangement, as illustrated in FIG. 24, the smoking substitute apparatus a150b comprises a bypass passage a180 which is fully separate from the vaporizer air passage a170. The smoking substitute apparatus therefore comprises bypass inlets a182 which are separate from the device inlets a176. The bypass passages a180 extend between the bypass inlets a177 and the bypass outlets a184. Since no mixing of the bypass airflow and vaporizer airflow occurs within the smoking substitute apparatus a150b, the same advantage is conveyed as the first embodiment with regards to reduced vapor cooling and potential condensation formation within the smoking substitute apparatus a150b. Providing separate bypass inlets a182 for the smoking substitute apparatus a150b may allow greater control over the pressure drop and resistance to draw of the bypass air passage a180. However, the advantages of a common inlet a176 outlined above for the first embodiment do not apply to this reference arrangement.

In the third reference arrangement, as illustrated in FIG. 25, the smoking substitute apparatus a150c comprises a bypass passage a180 which is fully enclosed within the smoking substitute apparatus a150c. Therefore, the bypass passage a180 shares a common inlet a176 and common outlet a174 with the vaporizer air passage a170. This reference arrangement may provide simpler manufacturing and more consistent airflow when compared to the first embodiment, since the number of external openings of the housing of the smoking substitute apparatus a150c is reduced. However, while the joining of the bypass airflow and vaporizer airflow within the smoking substitute apparatus a150c may allow for improved mixing of the airflow, cooling of the vapor within the device may also be increased, which may cause condensation to form within the bypass passage a180 and/or the vaporizer passage a170.

In the fourth reference arrangement, as illustrated in FIG. 26, the smoking substitute apparatus a150d comprises bypass air inlets a182 separate from the device air inlets a176. The bypass passage a180 joins with the vaporizer passage a170 upstream of the outlet a174 to form a single outlet airflow. As with the second reference arrangement, the advantages provided by a common inlet a176 are not present in this reference arrangement. Furthermore, the same considerations apply as for the third reference arrangement with regards to vapor mixing and cooling within the smoking substitute apparatus a150d.

The experimental work presented below is relevant to the embodiments disclosed above in particular in view of the consideration of air flow rate at the wick (and therefore air velocity at the wick in these embodiments), affected by the provision of a bypass airflow. Furthermore, the experimental work is relevant in the context of a consideration of turbulence at the wick, affected by the provision of flow conditioning upstream of the wick.

Development B

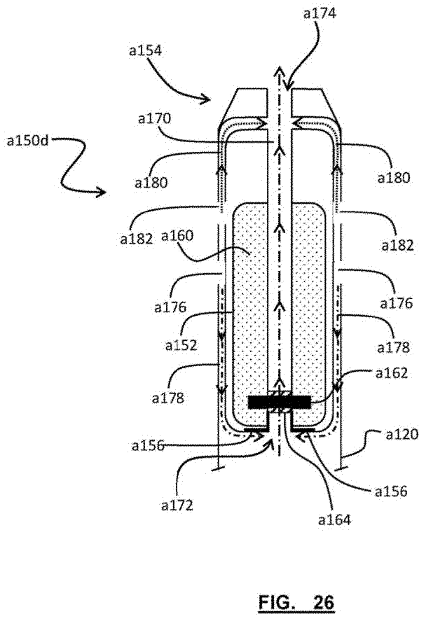

FIG. 27 shows a schematic heat shield arrangement b200 of a smoking substitute apparatus according to an embodiment of the disclosure. Components and parts of the apparatus of this embodiment that are common to the first reference arrangement of FIG. 19 are referenced with the same number (except for a “b” prefix), and are not necessarily discussed further in view of this embodiment.

The heat shield arrangement b200 includes an enclosure b201 formed primarily of a plastic material, and two heat shield plates b202a, b202b. The plates b202a, b202b are made of a material which has a thermal degradation temperature significantly higher than that of the plastic material which forms the enclosure b201. In this disclosure, the term “thermal degradation temperature” is used to describe the lowest temperature at which a material undergoes melting, softening, corrosion, spalling or combustion. The plates b202a, b202b are thus preferably made of a metal, ceramic, thermosetting polymer, or a composite thereof, and preferably have a thermal degradation temperature which is at least 100° C. higher than that of the plastic enclosure material. The plates b202a, b202b are configured to fit into and be held by grooves defined by the enclosure b201. The heater b164 of this arrangement is disposed so as to be between the plates b202a, b202b when the plates are held by the grooves, but is not illustrated in FIG. 27.

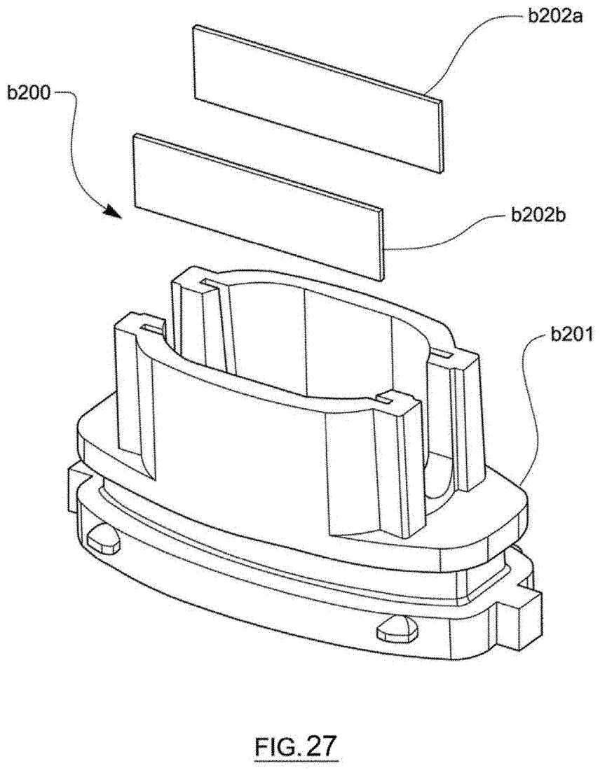

The superior thermal degradation properties of the plates b202a, b202b allow them to protect the plastic enclosure b201 from excessive heating by the heater b164. Specifically, when heat energy radiates from the heater b164 towards the enclosure b201, a significant portion of the heat energy is intercepted and absorbed by the plates b202a, b202b, reducing the amount of heat energy available to heat the plastic material of the enclosure b201. This reduces the risk of the enclosure b201 reaching a temperature which would cause thermal degradation of the plastic material. Therefore, the enclosure b201 is less likely to deform due to heating and alter the intended air flow through the apparatus, and there is a reduced risk of harmful plastic matter becoming entrained in the air flow and into the lungs of the user, for example due to melting of the enclosure b201. The heat shield plates b202a, b202b thus allow the enclosure b201 and the heater b164 to be placed closer together than a corresponding case in which the plates are absent, making the apparatus compact and easy to handle. To ensure that the heat radiating from the heater b164 is intercepted and absorbed by the heat shield plates, each plate b202a, b202b in the present embodiment presents to the heater b164 a heat-absorbing surface having an area of at least twice as large as a plan view projection of the heater onto the respective plate. This concept is illustrated schematically in FIG. 28, which shows a plan view projection of a heater b164 and a wick b162 onto a heat-absorbing surface of plate b202a. It is clear from FIG. 28 that the area of the heat-absorbing surface of plate b202a is at least twice as large as the plan view projection of the heater b164 onto the surface. In the present embodiment, the heat-absorbing surface of each heat shield plate b202a, b202b is also at least 20 mm², but may be at least 30 mm², or at least 40 mm².

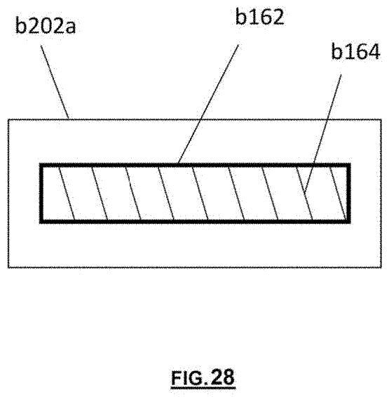

A more detailed schematic of a cross section of a further heat shield arrangement b300 of a smoking substitute apparatus according to an embodiment of the disclosure is illustrated in FIG. 29. Components and parts of the apparatus of this embodiment that are common to the first reference arrangement of FIG. 19 are referenced with the same number (except with a “b” prefix), and may not necessarily be discussed further in view of this embodiment.

FIG. 29 shows a cross sectional view of an enclosure b301 and heat shield plates b302a, b302b which are similar to those described in relation to FIG. 27. Also shown is a heater b164 of the apparatus wound around a wick b162, preferably in a helical manner. The plates b302a, b302b are disposed between the heater b164 and the enclosure b301 on opposing sides of the heater b164. This ensures the enclosure b301 is protected from both sides of the heater b164. Moreover, the enclosure b301 has a closest distance to the heater b164 of 2 mm, such that the size of the apparatus may be kept low while the heat shield plates b302a, b302b protects the enclosure b301 from thermal degradation.

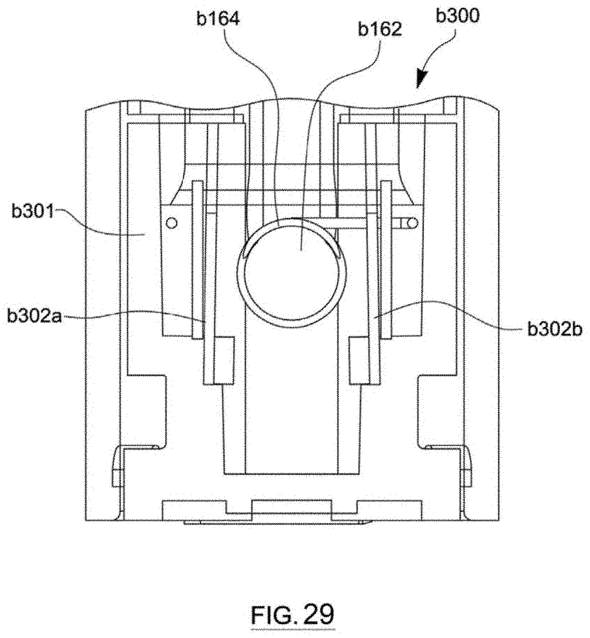

FIG. 30 illustrates a second perspective of the further heat shield arrangement b300 of FIG. 29. In FIG. 30 the shape of the enclosure b301 can be more clearly seen. The enclosure b301 defines a vaporization chamber, which has an elongate shape, wider in a first direction orthogonal to a longitudinal axis of the apparatus compared with a second direction orthogonal to the first direction and to the longitudinal axis of the apparatus. The heater b164 extends along the first direction and the plates b302a, b302b extend between the heater b164 and the enclosure b301 substantially parallel to the first direction. The wider first direction allows the heater b164 to be appropriately sized so as to sufficiently vaporize the aerosol precursor, while the narrower second direction provides a user-acceptable apparatus size and shape format and is possible due to the protecting characteristics of the plates b302a, b302b described previously. Also visible in FIG. 30 is a plastic housing b303. The plastic housing b303 allows the apparatus to be light-weight and cheap to manufacture, and provides the apparatus with a user-acceptable external format.

In other embodiments, there may be provided a single heat shield extending fully or partially around the heater. Such a heat shield may have any one or a combination of the features described in relation to any of plates b202a, b202b, b302a, b302b.

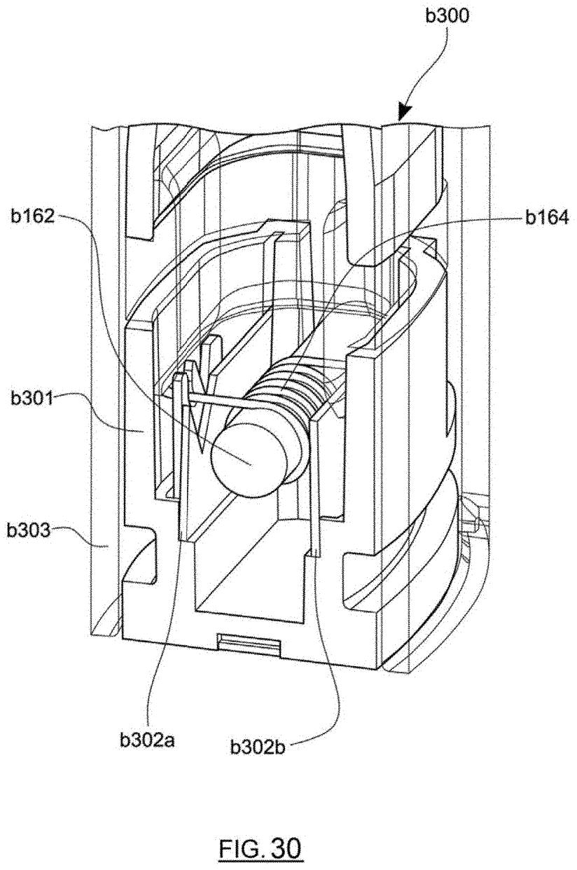

An apparatus according to the disclosure is configured such that in use, at least part of the air flow drawn by a user through the apparatus from the air inlet to the outlet bypasses the vaporization chamber defined by the enclosure. A second reference arrangement of an apparatus, shown in FIG. 31, provides an example of how such a bypassing air flow may be created. Accordingly, some embodiments of the disclosure may include one or a combination of the features of the second reference arrangement (and variations thereof) where such features are combinable with the present disclosure. This second reference arrangement is described below.

FIG. 31 illustrates a schematic longitudinal cross sectional view of a second reference arrangement of the smoking substitute apparatus forming part of the smoking substitute system shown in FIGS. 17 and 18. The arrangement illustrated in FIG. 31 differs from the first reference arrangement illustrated in FIG. 19 in that the substitute smoking apparatus includes two bypass passages b180 in addition to the vaporizer passage b170. The bypass air passages extend between the plurality of device air inlets b176 and two outlets b184. In other variations of the second reference arrangement, the number of bypass passages b180 and corresponding outlets b184 may be greater or smaller than in the illustrated example. Furthermore, there may be more or fewer air inlets and there may be more or fewer outlets.

In FIG. 31 for simplicity, the bypass passage b180 is shown with a substantially circular cross-sectional profile with a constant diameter along its length. In some variations of the second reference arrangement, the bypass passage b180 may have other cross-sectional profiles, such as oval shaped or polygonal shaped profiles. Further, in some variations of the second reference arrangement, the cross sectional profile and the diameter (or hydraulic diameter) of the bypass passage b180 may vary along its longitudinal axis.

The provision of a bypass passage b180 means that a part of the air drawn through the smoking substitute apparatus b150a when a user inhales via the mouthpiece b154 is not drawn through the vaporization chamber. This has the effect of reducing the flow rate through the vaporization chamber in correspondence with the respective flow resistances presented by the vaporizer passage b170 and the bypass passage b180. This can reduce the correlation between the flow rate through the smoking substitute apparatus b150a (i.e., the user's draw rate) and the particle size generated when the e-liquid b160 is vaporized and subsequently forms an aerosol. Therefore, the smoking substitute apparatus b150a of the second reference arrangement can deliver a more consistent aerosol to a user.

Furthermore, the smoking substitute apparatus b150a of the second reference arrangement is capable of producing an increased particle droplet size, d50, based on typical inhalation rates undertaken by a user, compared to the first reference arrangement of FIG. 19. Such larger droplet sizes may be beneficial for the delivery of vapor to a user's lungs. The preferred ratio between the dimensions of the bypass passage b180 and the dimensions of the vaporizer passage b170, and hence flow rate in the respective passages may be determined from representative user inhalation rates and from the required air flow rate through the vaporization chamber to deliver a desired droplet size. For example, an average total flow rate of 1.3 liters per minute may be split such that 0.8 liters per minute passes through the bypass air channel b180, and 0.5 liters per minute passes through the vaporizer channel b170, a bypass:vaporizer flow rate ratio of 1.6:1. Such a flow rate may provide an average droplet size, d50, of 1-3 μm (more preferably 2-3 μm) with a span of not more than 20 (preferably not more than 10). Alternative flow rate ratios may be provided based on calculations and measurements of user flow rate, vaporizer flow rate, and average droplet size d50. A bypass:vaporizer flow rate ratio of between 0.5:1 and 20:1, typically at an average total flow rate of 1.3 liters per minute may be advantageous depending on the configuration of the smoking substitute apparatus.

The bypass passage and vaporizer passage extend from a common device inlet b176. This has the benefit of ensuring more consistent airflow through the bypass passage b180 and vaporizer passage b170 across the lifetime of the smoking substitute apparatus b150a, since any obstruction that impinges on an air inlet b176 will affect the airflow through both passages equally. The impact of inlet manufacturing variations can also be reduced for the same reason. This can therefore improve the user experience for the smoking substitute apparatus b150a. Furthermore, the provision of a common device inlet b176 simplifies the construction and external appearance of the device.

The bypass passage b180 and vaporizer passage b170 separate upstream of the vaporization chamber. Therefore, no vapor is drawn through the bypass passage b180. Furthermore, because the bypass passage leads to outlet b184 that is separate from outlet b174 of the vaporizer passage, substantially no mixing of the bypass air and vaporizer air occurs within the smoking substitute apparatus b150a. Such mixing could otherwise lead to excessive cooling of the vapor and hence a build-up of condensation within the smoking substitute apparatus b150a. Such condensation could have adverse implications for delivering vapor to the user, for example by causing the user to draw liquid droplets rather than vapor when “puffing” on the mouthpiece b154.

Having a part of the air flow bypass the vaporization chamber results in the capacity for the air flow to cool the enclosure/vaporization chamber being lower than in a corresponding case in which no bypass occurs. Thus, the heat shield of the disclosure accommodates the reduced cooling present in the apparatus, lowering the risk of thermal degradation of the enclosure which may otherwise occur.

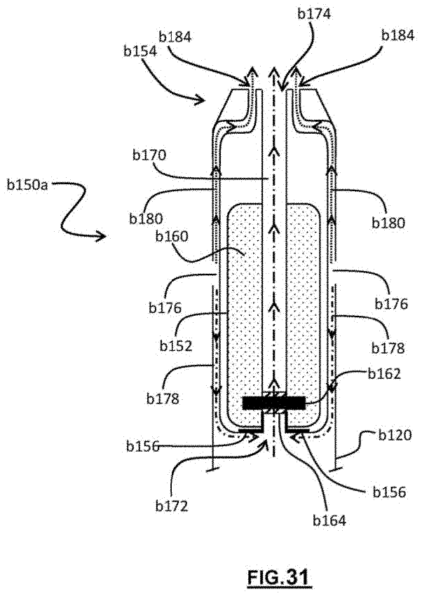

A further example of a bypass air flow is presented by a third reference arrangement. Accordingly, in some embodiments, the apparatus may include one or a combination of features of a third reference arrangement (and variations thereof), shown schematically in FIG. 32, where such features are combinable with the present disclosure. This third reference arrangement is described below.

FIG. 32 illustrates a longitudinal cross sectional view of a consumable b250 according to a further arrangement. In FIG. 32, the consumable b250 is shown attached, at a first end of the consumable b250, to the main body b120 (similar to main body 120 of FIG. 17 and FIG. 18). More specifically, the consumable b250 is configured to engage and disengage with the main body b120 and is interchangeable with the first reference arrangement 150 as shown in FIGS. 19 and 20. Furthermore, the consumable b250 is configured to interact with the main body b120 in the same manner as the first reference arrangement 150 and the user may operate the consumable b250 in the same manner as the first reference arrangement 150.

The consumable b250 comprises a housing. The consumable b250 comprises an aerosol generation chamber b280 in the housing. As shown in FIG. 32, the aerosol generation chamber b280 takes the form of an open ended container, or a cup, with a single chamber outlet b282 opened towards the outlet b274 of the consumable b250.

In the illustrated third reference arrangement, the housing has a plurality of air inlets b272 defined or opened at the sidewall of the housing. An outlet b274 is defined or opened at a second end of the consumable b250 that comprises a mouthpiece b254. A pair of passages b270 each extend between the respective air inlets b272 and the outlet b274 to provide flow passage for an air flow b412 as a user puffs on the mouthpiece b254. The chamber outlet b282 is configured to be in fluid communication with the passages b270. The passages b270 extend from the air inlets b272 towards the first end of the consumable b250 before routing back to towards the outlet b274 at the second end of the consumable b250. That is, a portion of each of the passages b270 axially extends alongside the aerosol generation chamber b280. The path of the air flow path b412 is illustrated in FIG. 32. In variations of the third reference arrangement, the passages b270 may extend from the air inlet b272 directly to the outlet b274 without routing towards the first end of consumable b250, e.g., the passages b270 may not axially extend alongside the aerosol generation chamber b280.

In some other variations of the third reference arrangement, the housing may not be provided with any air inlet for an air flow to enter the housing. For example, the chamber outlet may be directly connected to the outlet of the housing by an aerosol passage and therefore said aerosol passage may only convey aerosol as generated in the aerosol generation chamber. In these variations, the discharge of aerosol may be driven at least in part by the pressure increase during vaporization of aerosol form.

Referring back to the third reference arrangement of FIG. 32, the chamber outlet b282 is positioned downstream from the heater in the direction of the vapor and/or aerosol flow b414 and serves as the only gas flow passage to the internal volume of the aerosol generation chamber b280. In other words, the aerosol generation chamber b280 is sealed against air flow except for having the chamber outlet b282 in communication with the passages b270, the chamber outlet b282 permitting, in use, aerosol generated by the heater to be entrained into an air flow along the passage b270. In some other variations of the third reference arrangement, the sealed aerosol generation chamber b280 may comprise a plurality of chamber outlets b282 each arranged in fluid commutation with the passages b270. In the illustrated third reference arrangement, the aerosol generation chamber b280 does not comprise any aperture upstream of the heater that may serve as an air flow inlet (although in some arrangements a vent may be provided). In contrast with the consumable 150 as shown in FIGS. 19 and 20, the passages b270 of the consumable b250 allow the airflow, e.g., an entire amount of air flow, entering the housing to bypass the aerosol generation chamber b280. Such arrangement allows aerosol precursor to be vaporized in absence of the air flow. Therefore, the aerosol generation chamber may be considered to be a “stagnant” chamber. For example, the volumetric flowrate of vapor and/or aerosol in the aerosol generation chamber is configured to be less than 0.1 litre per minute. The vaporized aerosol precursor may cool and therefore condense to form an aerosol in the aerosol generation chamber b280, which is subsequently expulsed into or entrained with the air flow in passages b270. In addition, a portion of the vaporized aerosol precursor may remain as a vapor before leaving the aerosol generation chamber b280, and subsequently forms an aerosol as it is cooled by the air flow in the passages b270. The flow path of the vapor and/or aerosol b414 is illustrated in FIG. 32.

In the illustrated third reference arrangement, the chamber outlet b282 is configured to be in fluid communication with a junction b290 at each of the passages b270 through a respective vapor channel b292. The junctions b290 merge the vapor channels b292 with their respective passages b270 such that vapor and/or aerosol formed in the aerosol generation chamber b280 may expand or entrain into the passages b270 through junction inlets of said junctions b290. The vapor channels form a buffering volume to minimize the amount of air flow that may back flow into the aerosol generation chamber b280. In some other variations of the third reference arrangement (not illustrated), the chamber outlet b282 may directly open towards the junction b290 at the passage, and therefore in such variations the vapor channel b292 may be omitted.

In some variations of the third reference arrangement (not illustrated), the chamber outlet may be closed by a one-way valve. Said one way valve may be configured to allow a one-way flow passage for the vapor and/or aerosol to be discharged from the aerosol generation chamber, and to reduce or prevent the air flow in the passages from entering the aerosol generation chamber.

In the illustrated third reference arrangement, the aerosol generation chamber b280 is configured to have a length of 20 mm and a volume of 680 mm³. The aerosol generation chamber is configured to allow vapor to be expulsed through the chamber outlet at a rate greater than 0.1 mg/second. In other variations of the third reference arrangement the aerosol generation chamber may be configured to have an internal volume ranging between 68 mm³ to 680 mm³, wherein the length of the aerosol generation chamber may range between 2 mm to 20 mm.

As shown in FIG. 32, a part of each of the passages b270 axially extends alongside the aerosol generation chamber b280. For example, the passages b270 are formed between the aerosol generation chamber b280 and the housing. Such an arrangement reduces heat transfer from the aerosol generation chamber b280 to the external surfaces of the housing.

The aerosol generation chamber b280 comprises a heater extending across its width. The heater comprises a porous wick b262 and a heating filament b264 helically wound around a portion of the porous wick b262. A tank b252 is provided in the space between the aerosol generation chamber b280 and the outlet b274, the tank being for storing a reservoir of aerosol precursor. Therefore, in contrast with the reference arrangement as shown in FIGS. 19 and 20, the tank b252 in the third reference arrangement does not substantially surround the aerosol generation chamber nor the passage b270. Instead, as shown in FIG. 32, the tank is substantially positioned above the aerosol generation chamber b280 and the porous wick b262 when the consumable b250 is placed in an upright orientation during use. The end portions of the porous wick b262 each extend through the sidewalls of the aerosol generation chamber b280 and into a respective liquid conduit b266 which is in fluid communication with the tank b252. The wick b262, saturated with aerosol precursor, may prevent gas flow passage into the liquid conduits b266 and the tank b252. Such an arrangement may allow the aerosol precursor stored in the tank b252 to convey towards the porous wick b262 through the liquid conduits b266 by gravity. The liquid conduits b266 are configured to have a hydraulic diameter that allow a controlled amount of aerosol precursor to flow from the tank b252 towards the porous wick b262. More specifically, the size of liquid conduits b266 are selected based on the rate of aerosol precursor consumption during vaporization. For example, the liquid conduits b266 are sized to allow a sufficient amount of aerosol precursor to flow towards and replenish the wick, yet not so large as to cause excessive aerosol precursor to leak into the aerosol generation chamber. The liquid conduits b266 are configured to have a hydraulic diameter ranging from 0.01 mm to 10 mm or 0.01 mm to 5 mm. Preferably, the liquid conduits b266 are configured to have a hydraulic diameter in the range of 0.1 mm to 1 mm.

The heating filament is electrically connected to electrical contacts b256 at the base of the aerosol generation chamber b280, sealed to prevent air ingress or fluid leakage. As shown in FIG. 32, when the first end of the consumable b250 is received into the main body b120, the electrical contacts b256 establish electrical communication with corresponding electrical contacts of the main body b120, and thereby allow the heater to be energized.

The vaporized aerosol precursor, or aerosol in the condensed form, may discharge from the aerosol generation chamber b280 based on pressure difference between the aerosol generation chamber b280 and the passages b270. Such pressure difference may arise form i) an increased pressure in the aerosol generation chamber b280 during vaporization of aerosol form, and/or ii) a reduced pressure in the passage during a puff.

For example, when the heater is energized and forms a vapor, it expands in to the stagnant cavity of the aerosol generation chamber b280 and thereby causes an increase in internal pressure therein. The vaporized aerosol precursor may immediately begin to cool and may form aerosol droplets. Such increase in internal pressure causes convection inside the aerosol generation chamber which aids expulsing aerosol through the chamber outlet b282 and into the passages b270.

In the illustrated third reference arrangement, the heater is positioned within the stagnant cavity of the aerosol generation chamber b280, e.g., the heater is spaced from the chamber outlet b282. Such arrangement may reduce or prevent the amount of air flow entering the aerosol generation chamber, and therefore it may minimize the amount of turbulence in the vicinity of the heater. Furthermore, such arrangement may increase the residence time of vapor in the stagnant aerosol generation chamber b280, and thereby may result in the formation of larger aerosol droplets. In some other variations of the third reference arrangement, the heater may be positioned adjacent to the chamber outlet and therefore that the path of vapor b414 from the heater to the chamber outlet b282 is shortened. This may allow vapor to be drawn into or entrained with the air flow in a more efficient manner.

The junction inlet at each of the junctions b290 opens in a direction orthogonal or non-parallel to the air flow. That is, the junction inlet each opens at a sidewall of the respective passages b270. This allows the vapor and/or aerosol from the aerosol generation chamber b280 to entrain into the air flow at an angle, and thus improving localized mixing of the different streams, as well as encouraging aerosol formation. The aerosol may be fully formed in the air flow and be drawn out through the outlet at the mouthpiece.

With the absence of, or much reduced, air flow in the aerosol generation chamber, the aerosol as generated by the illustrated third reference arrangement has a median droplet size d₅₀ of at least 1 μm. More preferably, the aerosol as generated by the illustrated third reference arrangement has a median droplet size d₅₀ of ranged between 2 μm to 3 μm.

In embodiments in which one or a combination of the features of the third reference arrangement of FIG. 32 are incorporated, the heat shield plates b202a, b202b, or a single heat shield, are particularly advantageous. For example, the provision of a “stagnant” chamber almost completely eliminates air flow over the heater. Thus, the stagnant chamber significantly decreases the cooling effect that would otherwise be provided to the enclosure b201 by an air flow through the vaporization chamber. Thus, in such cases, the heat shield plates b202a, b202b are particularly beneficial in protecting the enclosure b201 from high levels of excess heat energy which are not transported away by the air flow.

The experimental results reported below are relevant to the embodiments disclosed above in view of their demonstration of the control over particle size based on control of the conditions at the wick. In particular, the embodiments disclosed above have an effect on the temperature in the vaporization chamber, in view of the effect of the heat shield and/or the provision of bypass airflow.

Development C

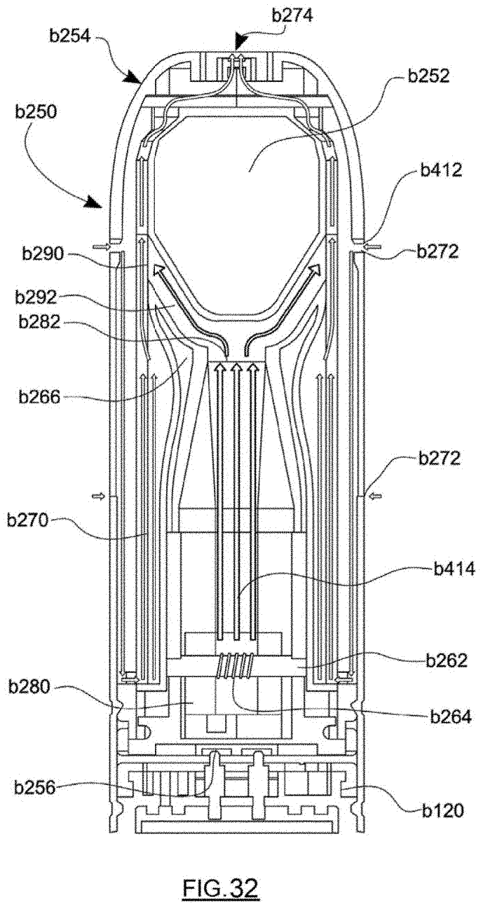

FIG. 33 illustrates a smoking substitute apparatus according to an embodiment of the disclosure. Components and parts of the apparatus that are common to the reference arrangement of FIG. 19 are referenced with the same number (but with a “c” prefix where appropriate), and are not discussed further in view of this embodiment.

FIG. 33 is a schematic longitudinal cross section of a smoking substitute apparatus c200 according to the present embodiment of the disclosure. The apparatus includes a housing, having a first end c201 and a second end c202 connected by sidewalls of the housing. Air inlets c210 are disposed at the sidewalls of the housing, and are configured to allow ingress of air into the apparatus c200.

A user can induce ingress of air into the apparatus, via the air inlets, by inhaling at an outlet c174 of the apparatus, disposed at the second end c202 of the housing. The outlet c174 and the air inlets c210 are in fluid communication with one another, and hence by inhaling at the outlet c174, the user draws air from the apparatus c200, causing a pressure drop which induces air to be drawn into the apparatus through the air inlets c210.

As air ingresses through each air inlet c210, a T-junction c220 disposed near, or formed by, the air inlet splits the air into a main airflow and a bypass airflow. The main airflow is directed along a main channel c230 in which the porous wick c162 is disposed, and passes over the wick c162, entraining aerosol droplets and exiting the apparatus via the outlet c174. The bypass air flow is directed along a bypass channel c240. The air flowing through the bypass channel c240 bypasses the wick c162 and does not entrain any aerosol droplets before exiting through the outlet c174. The dashed arrows in FIG. 33 show the directions in which the air in the main and bypass channels c230, 240 flows when a user inhales from the outlet c174. In the present embodiment, the air flowing along the main flow channel c230 is initially directed towards the first end c201 of the housing, away from the outlet c174, and the air flowing along the bypass flow channel c240 is initially directed towards the outlet c174 at the second end of the housing c202. The air flowing along the main channel c230 turns through 180° once before reaching the outlet such that the main air flow downstream of the aerosol generator is in the opposite direction to the main air flow upstream of the aerosol generator and proximate the air inlet. In other embodiments, the arrangement may be reversed, such that the main channel initially directs air towards the second end of the housing c202, while the bypass channel initially directs air towards the first end c201.

The advantages of this arrangement are two-fold. Firstly, the provision of bypass channels c240 allows users to inhale from the outlet c174 with higher rates of air flow than would otherwise be the case. This is because the higher rate of air flow drawn from the outlet c174 is split between the main flow channel c230 and the bypass flow channels c240, and thus the rate of air flow through the main channel c230 may remain low enough so as to generate aerosol droplets having a size distribution that optimizes nicotine delivery to the user, such as a median droplet size, d₅₀, of at least 1 μm.

The second advantage of this arrangement is due to the directions in which the air in the main and bypass channels c230, c240 flow. The ratio of air flow drawn through the main flow channel c230 and the bypass flow channel c240, and hence the size distribution of aerosol droplets entrained in the air flow of the main flow channel c230, is in part determined by the respective resistances to flow in each channel c230, c240. A factor in the resistance to flow of a channel is the total length of the channel itself—i.e., longer channels have larger resistances to flow than shorter channels. Hence, the ratio of air flow drawn through the main and bypass channels can be determined by the respective lengths of the main and bypass flow channels.

Directing the initial air flow of the main flow channel c230 away from the outlet c174, and that of the bypass flow channels c240 towards the outlet c174, as illustrated in FIG. 33, provides a difference between the lengths of the main and bypass flow channels c230, c240, as both channels are configured to end at the outlet c174. This achieves a difference in flow resistance between the channels c230, c240, and this difference can be set to allow the apparatus c200 to be tailored such that users with different inhalation flow rates receive adequately-sized aerosol droplets providing efficient nicotine delivery. For example, the relative resistances may be varied between apparatuses by having the inlets c210 disposed at different points along the sidewalls, or moving a turning point of the main flow channel c230 to a different position.

Additionally, the difference in lengths, and hence the difference in flow resistances, is achieved in a spatially and cost efficient arrangement by having the inlet c210 along a sidewall and having one 180° turn in the main flow channel c230. As a counterexample, if the respective air flows of the main and bypass channels c230, c240 were not directed away from each other in the manner described above, one or both of the channels may be required to have a convoluted path for air to flow along, if significant length differences were to be achieved. This would increase manufacture costs and require a larger space when compared to the arrangement of the present disclosure.

A further advantage of the present arrangement is provided by having the air inlets c210 located at the sidewalls. Aerosol may become attached to the walls of the main flow channel c230 while travelling along the main flow channel c230 between the wick c162 and the outlet c174. When this occurs, this “condensed” aerosol may move under the influence of gravity. When the apparatus is held upright, with the outlet at the top, these drops may flow towards the first end c201 of the housing. In a comparative apparatus which has an air inlet formed at the first end c201 of the apparatus, these drops may leak from the air inlet. Similarly, aerosol precursor may leak from the wick c162 and also leak from the air inlet. However, in the preferred embodiments of the disclosure, because the air inlet is formed at the side wall, condensed aerosol and leaking precursor are less likely to reach the air inlet in order to leak from the apparatus.

The experimental work reported below is relevant to the embodiments disclosed above in view of the effect shown by which the air flow conditions at the wick have an influence on the particle size of the generated aerosol. The provision of a bypass airflow affects the air flow conditions at the wick.

Development D

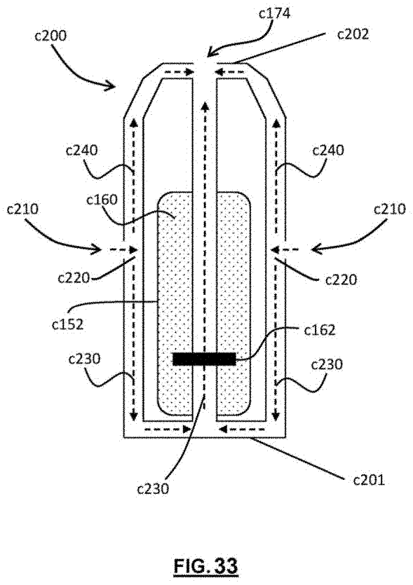

FIG. 34 illustrates a schematic longitudinal cross sectional view of a first embodiment of the smoking substitute apparatus forming part of the smoking substitute system shown in FIGS. 17 and 18. The embodiment illustrated in FIG. 34 differs from the reference arrangement illustrated in FIG. 19 in that the substitute smoking apparatus includes two bypass passages d180 in addition to the vaporizer passage d170. The bypass passages d180 further include an auxiliary heater d190 for heating the air passing through the bypass passage d180. The bypass air passages d180 extend between the plurality of device air inlets d176 and two outlets d184. In other embodiments, the number of bypass passages d180 may be greater or smaller than in the illustrated example.

In FIG. 34, for simplicity, the bypass passage d180 is shown with a substantially circular cross-sectional profile with a constant diameter along its length. In some embodiments, the bypass passage d180 may have other cross-sectional profiles, such as oval shaped or polygonal shaped profiles. Further, in some embodiments, the cross sectional profile and the diameter (or hydraulic diameter) of the bypass passage d180 may vary along its longitudinal axis.

The provision of a bypass passage d180 means that a part of the air drawn through the smoking substitute apparatus d150a when a user inhales via the mouthpiece d154 is not drawn through the vaporization chamber. This has the effect of reducing the flow rate through the vaporization chamber in correspondence with the respective flow resistances presented by the vaporizer passage d170 and the bypass passage d180. This can reduce the correlation between the flow rate through the smoking substitute apparatus d150a (i.e., the user's draw rate) and the particle size generated when the e-liquid d160 is vaporized and subsequently forms an aerosol. Therefore, the smoking substitute apparatus d150a of the present embodiment can deliver a more consistent aerosol to a user.

Furthermore, the smoking substitute apparatus d150a of the present embodiment is capable of producing an increased particle droplet sizes, d₅₀, based on typical inhalation rates undertaken by a user, compared to the reference arrangement of FIG. 19. Such larger droplet sizes may be beneficial for the delivery of vapor to a user's lungs. The preferred ratio between the dimensions of the bypass passage d180 and the dimensions of the vaporizer passage d170, and hence flow rate in the respective passages may be determined from representative user inhalation rates and from the required air flow rate through the vaporization chamber to deliver a desired droplet size. For example, an average total flow rate of 1.3 liters per minute may be split such that 0.8 liters per minute passes through the bypass air channel 180, and 0.5 liters per minute passes through the vaporizer channel d170, a bypass:vaporizer flow rate ratio of 1.6:1. Such a flow rate may provide a droplet size, d₅₀, of 1-3 μm (more preferably 2-3 μm) with a span of not more than 20 (preferably not more than 10). Alternative flow rate ratios may be provided based on calculations and measurements of user flow rate, vaporizer flow rate, and average droplet size d₅₀. A bypass:vaporizer flow rate ratio of between 0.5:1 and 20:1, typically at an average total flow rate of 1.3 liters per minute may be advantageous depending on the configuration of the smoking substitute apparatus.

The auxiliary heater d190 for heating air passing through the bypass passage d180 is provided so as to reduce any cooling effect from the bypass air flow on the combined air flow (i.e., the combination of the bypass air flow and the main air flow) from the smoking substitute apparatus d150a. Such cooling might otherwise result in a lower than expected vapor temperature for the user, which may be undesirable, and may adversely effect, for example, the average droplet size, d₅₀, or other characteristics of the aerosol.

The auxiliary heater d190 for heating air may comprise one or more heating elements forming a bypass air heater d190 arranged within the bypass passage d180. In this embodiment, the bypass air heater d190 comprises an electrically heatable mesh located in the airflow stream. The mesh may be formed of a material that is heatable by resistive heating using an electrical current. In other embodiments, the heater d190 may comprise a plurality of meshes. In still further embodiments, the bypass air heater d190 may comprise other forms of heating element, such as a heating coil or heating plate. The bypass air heater d190 may be arranged within the bypass passage d180. Alternatively, the bypass air heater d190 may be arranged adjacent to the bypass passage. The bypass air heater d190 may define part of the bypass passage d180 (i.e., the heater may form a part of the wall of the bypass passage d180). The bypass air heater d190 may comprise a heating element arranged externally of the bypass passage d180, with the heating element placed into thermal communication with the passage via a thermally conductive element.

The bypass air heater d190 may be operable in synchronism with the filament d164 of the vaporizer. The bypass air heater d190 may be operable in combination with the filament d164 of the vaporizer (i.e., there may be a time offset between the switch-on and switch-off operations of the bypass air heater d190 and the filament d164), and the bypass air heater d190 and the filament d164 may be operated for different periods of time. The bypass air heater d190 may be operable independently of the filament d164. The type of operation (e.g., the length of time delay) may be selectable by a user. The level of heating from the bypass air heater (i.e., the power supplied or heating time) may be selectable by a user. The smoking substitute apparatus d150a comprises a set of electrical contacts (not shown) separate from the electrical contacts for the filament d164 such that the bypass air heater d190 is independently electrically connectable to the power source. In alternative embodiments, a common set of electrical contacts may be used for both the filament d164 and the bypass air heater d190.

In alternative embodiments, the auxiliary heater d190 for heating air may comprise a thermally conductive element in thermal communication with the filament d164 of the vaporizer. Such a thermally conductive element conducts heat from the filament d164 to the bypass passage d180 to thereby heat the air passing through the bypass passage d180. In the first embodiment, the bypass passage d180 and vaporizer passage d170 extend from a common device inlet d176. This has the benefit of ensuring more consistent airflow through the bypass passage d180 and vaporizer passage d170 across the lifetime of the smoking substitute apparatus d150, since any obstruction that impinges on an air inlet d176 will affect the airflow through both passages equally. The impact of inlet manufacturing variations can also be reduced for the same reason. This can therefore improve the user experience for the smoking substitute apparatus d150. Furthermore, the provision of a common device inlet d176 simplifies the construction and external appearance of the device.

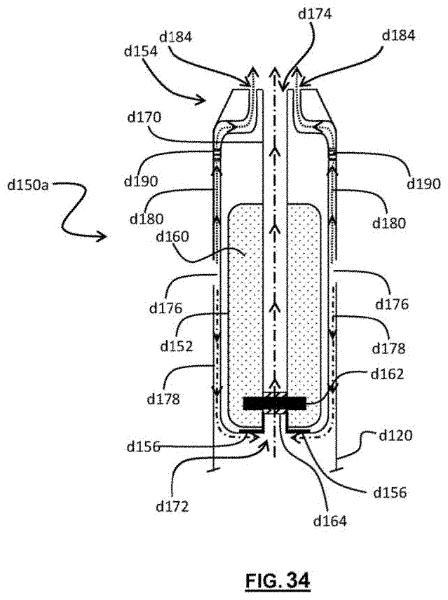

The second embodiment, as illustrated in FIG. 35, differs from the first embodiment in that the smoking substitute apparatus d150b comprises a bypass passage d180 which is fully separate from the vaporizer air passage d170. The smoking substitute apparatus therefore comprises bypass inlets d177 which are separate from the device inlets d176. The bypass passages d180 extend between the bypass inlets d177 and the bypass outlets d184. Providing separate bypass inlets d182 for the smoking substitute apparatus d150b may allow greater control over the pressure drop and resistance to draw of the bypass air passage d180.

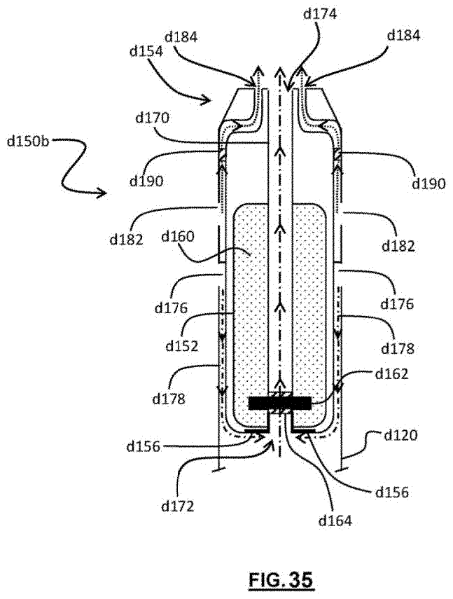

The third embodiment, as illustrated in FIG. 36, differs from the first embodiment in that the smoking substitute apparatus d150c comprises a bypass passage d180 which is fully enclosed within the smoking substitute apparatus d150c. The bypass passage d180 shares a common inlet d176 and common outlet d174 with the vaporizer air passage d170. In this embodiment, the bypass air heater d190 is arranged upstream of the joining point of the bypass passage d180 and the vaporizer passage d170. A smoking substitute apparatus d150c according to the third embodiment may provide simpler manufacturing and more consistent airflow when compared to the first embodiment, since the number of external openings of the housing of the smoking substitute apparatus d150c is reduced. Furthermore, combining the main air flow and the bypass air flow within the smoking substitute apparatus d150c may allow for improved mixing of the airflow compared to the arrangement of the first or second embodiments. For example, it prevents a user from affecting the bypass airflow by inadvertently blocking one or more of the bypass outlets.

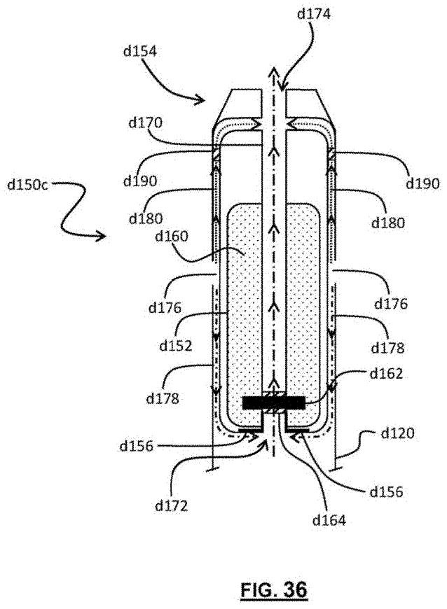

The fourth embodiment, as illustrated in FIG. 37, differs from the third embodiment in that the smoking substitute apparatus d150d comprises bypass air inlets d182 separate from the device air inlets d176. The bypass passage d180 joins with the vaporizer passage d170 upstream of the outlet d174 to form a single outlet airflow. As with the third embodiment, the bypass air heater d190 is arranged upstream of the joining point of the bypass passage d180 and the vaporizer passage d170.

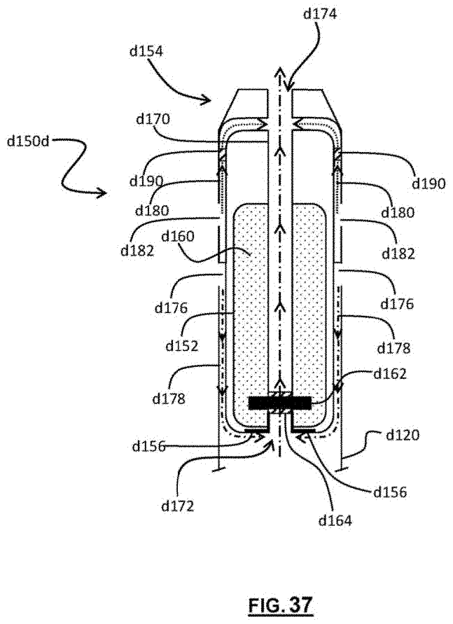

The smoking substitute apparatus d150a, d150b, d150c, d150d may further comprise a flow conditioning apparatus d200 arranged upstream of the wick d162 and filament d164 in the vaporizer passage d170, as illustrated in FIG. 38. The flow conditioning apparatus d200 is configured to reduce turbulence in the air flow through vaporizer passage d170 and past the wick d162 and filament d164. This may be advantageous for improving aerosol generation, and for reducing the spread in the aerosol particle size distribution. In FIG. 38, the turbulent air flow is indicated by the arrows d400. Once the airflow has passed through the flow conditioning apparatus d200, the turbulence is reduced, as indicated by the aligned arrows d402.

Where the flow conditioning apparatus d200 is provided in the smoking substitute apparatus d150a, d150b, d150c, d150d, the provision of a bypass passage d180 may reduce the effect of the flow conditioning apparatus d200 on the overall resistance to draw of the smoking substitute apparatus d150a, d150b, d150c, d150d, which may in turn increase the range of possible design parameters for the flow conditioning apparatus d200 allowing air flow past the wick d162 and filament d164 to be more readily optimized for improved aerosol generation without compromising the overall operation of the smoking substitute apparatus d150a, d150b, d150c, d150d.

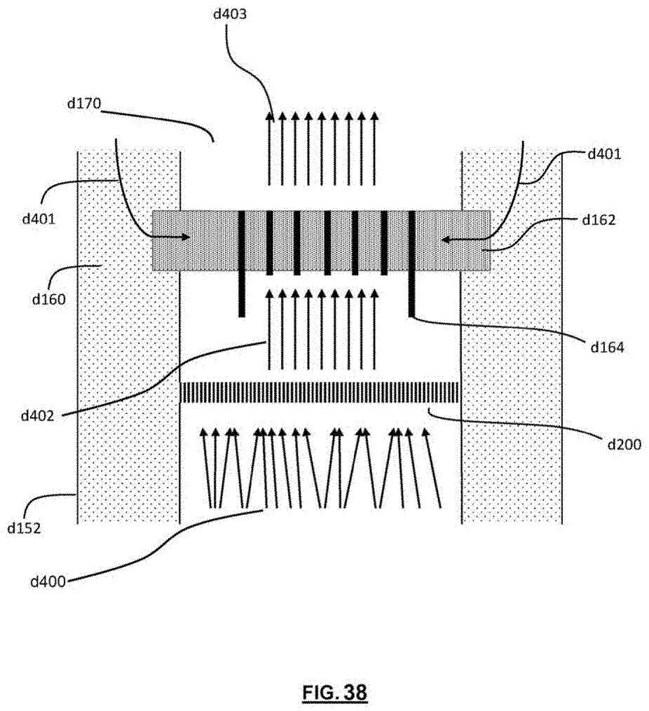

The flow conditioning apparatus d200 may, for example, comprise an air permeable mesh arranged across the vaporizer passage d170 such that air drawn through the vaporizer passage d170 is drawn through the mesh. The mesh may be constructed from an air-impermeable material comprising one or more bores d202 extending through, as illustrated exemplarily in FIG. 39.

The experimental work reported below is relevant to the embodiments disclosed above in view of the effect shown by which the air flow conditions at the wick have an influence on the particle size of the generated aerosol. The provision of a bypass airflow affects the air flow conditions at the wick.

Development E

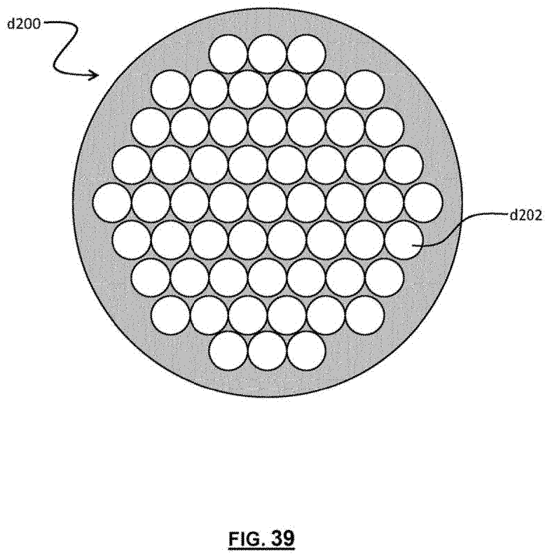

FIG. 40 illustrates a schematic longitudinal cross sectional view of a first embodiment of the smoking substitute apparatus forming part of the smoking substitute system shown in FIGS. 17 and 18. The embodiment illustrated in FIG. 40 differs from the reference arrangement illustrated in FIG. 19 in that the substitute smoking apparatus includes two bypass passages e180 in addition to the vaporizer passage e170. The bypass passages correspond to the second air passage of the claims. In other embodiments, the number of bypass passages may be greater or smaller than in the illustrated example.

The bypass air passages e180 extend from the same air inlet e176 as the vaporizer passage e170. The bypass passages e180 join with the vaporizer passage e170 at a junction e184, arranged proximal or adjacent to the mouthpiece e154 of the smoking substitute apparatus e150a and downstream of the heater e164 and wick e162. The bypass passages e180 join with the vaporizer passage e170 at the junction e184 in a direction orthogonal to the longitudinal axis of the vaporizer passage e170.

In FIG. 40, for simplicity, the bypass passage e180 is shown with a substantially circular cross-sectional profile with a constant diameter along its length. In some embodiments, the bypass passage e180 may have other cross-sectional profiles, such as oval shaped or polygonal shaped profiles. Further, in some embodiments, the cross sectional profile and the diameter (or hydraulic diameter) of the bypass passage e180 may vary along its longitudinal axis. The section of the bypass passages e180 close to the junction e184 may be shaped to direct airflow within the junction e184. For example, the bypass passage e180 may comprise a tapering to form a nozzle. The walls of the bypass passage e180 may be textured (e.g., roughened) to support generation of turbulent flow within the junction e184 which may aid mixing of the bypass airflow and main airflow.

The provision of a bypass passage e180 means that a part of the air drawn through the smoking substitute apparatus e150a when a user inhales via the mouthpiece e154 is not drawn through the vaporization chamber. This has the effect of reducing the flow rate through the vaporization chamber in correspondence with the respective flow resistances presented by the vaporizer passage e170 and the bypass passage e180. This can reduce the correlation between the flow rate through the smoking substitute apparatus e150 (i.e., the user's draw rate) and the particle size generated when the e-liquid e160 is vaporized and subsequently forms an aerosol. Therefore, the smoking substitute apparatus e150 of the present embodiment can deliver a more consistent aerosol to a user.

Furthermore, the smoking substitute apparatus e150 of the present embodiment is capable of producing an increased particle droplet sizes, d₅₀, based on typical inhalation rates undertaken by a user, compared to the reference arrangement of FIG. 19. Such larger droplet sizes may be beneficial for the delivery of vapor to a user's lungs. The preferred ratio between the dimensions of the bypass passage e180 and the dimensions of the vaporizer passage e170, and hence flow rate in the respective passages may be determined from representative user inhalation rates and from the required air flow rate through the vaporization chamber to deliver a desired droplet size. For example, an average total flow rate of 1.3 liters per minute may be split such that 0.8 liters per minute passes through the bypass air channel 180, and 0.5 liters per minute passes through the vaporizer channel e170, a bypass:vaporizer flow rate ratio of 1.6:1. Such a flow rate may provide an average droplet size, d₅₀, of 1-3 μm (more preferably 2-3 μm) with a span of note more than 20 (preferably not more than 10). Alternative flow rate ratios may be provided based on calculations and measurements of user flow rate, vaporizer flow rate, and average droplet size d₅₀. A bypass:vaporizer flow rate ratio of between 0.5:1 and 20:1, typically at an average total flow rate of 1.3 liters per minute may be advantageous depending on the configuration of the smoking substitute apparatus.

The bypass passage and vaporizer passage extend from a common device inlet e176. This has the benefit of ensuring more consistent airflow through the bypass passage e180 and vaporizer passage e170 across the lifetime of the smoking substitute apparatus e150, since any obstruction that impinges on an air inlet e176 will affect the airflow through both passages equally. The impact of inlet manufacturing variations can also be reduced for the same reason. This can therefore improve the user experience for the smoking substitute apparatus e150. Furthermore, the provision of a common device inlet e176 simplifies the construction and external appearance of the device.

In this first embodiment, where two bypass passages e180 are present, the bypass passages e180 meet at the junction e184 at symmetrical at opposing positions. As the bypass airflows combine with the main airflow, therefore, the opposing flow direction of the different airflows can aid mixing by providing a source of turbulent flow within the smoking substitute apparatus e150a. The location of the junction e184 proximal to the mouthpiece, meanwhile, reduces the distance that the airflow must travel within the device in a downstream direction of the junction e184. Therefore, the likelihood of condensation formation within the device resulting from vapor cooling by the bypass airflow can be reduced.

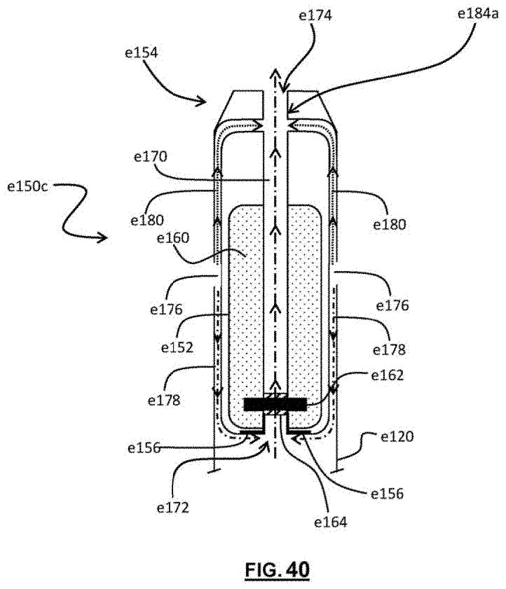

A second embodiment of the smoking substitute apparatus e150b is illustrated by FIG. 41. The second embodiment differs from the first embodiment in that the bypass passages e180 are offset from each other in a direction perpendicular to the longitudinal axis of the vaporizer passage e170 in terms of their meeting direction at the junction e184b. Arranging the bypass passages in this way leads to improved mixing between the bypass airflow and the main airflow, since it introduces a level of swirl or rotation in the airflow (e.g., the creation of a vortex), as indicated by the reference number e181a. The vorticity of the flow through the junction may, for example, be optimized according to expected user flow rates by adjusting the level of perpendicular offset between the outlets, the number of outlets, the and the angle between the outlet alignment and the wall of the vaporizer passage e170.

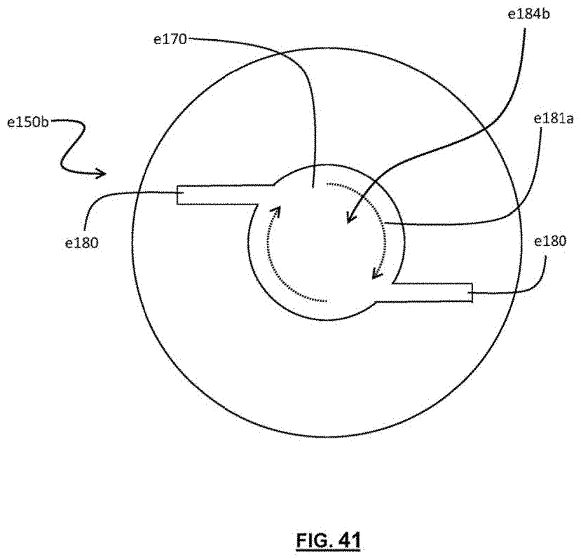

The mixing of the bypass airflow and the vaporizer airflow may be further enhanced by introducing an offset between the bypass passage outlets along the longitudinal axis of the vaporizer passage e170, in addition to, or in some cases as an alternative to, the perpendicular offset of the second embodiment, as illustrated in FIG. 42. In this arrangement, an extended mixing zone can be created, by arranging the outlets so as to create an extended vortex rotating in a single direction. Alternatively, counter-rotating vortices can be created along the vaporizer passage e170.

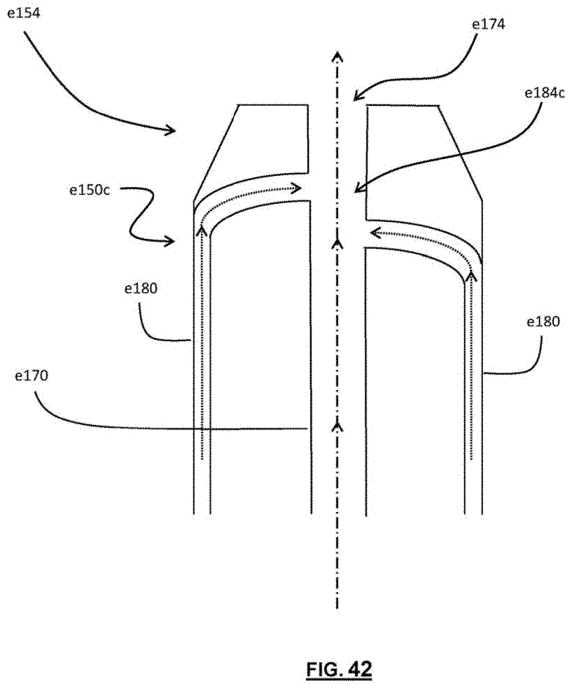

The smoking substitute apparatus may further comprise a flow conditioning apparatus e200 arranged upstream of the wick e162 and filament e164 in the vaporizer passage e170, as illustrated in FIG. 43. The flow conditioning apparatus e200 is configured to reduce turbulence in the air flow through the vaporization chamber and past the wick e162 and filament e164. This may be advantageous for improving aerosol generation, and for reducing the spread in the aerosol particle size distribution. Furthermore, the provision of a flow conditioning apparatus may generate a more consistent airflow along the vaporizer channel e170 and into the junction e184a, e184b, e184c. In FIG. 43, the turbulent air flow is indicated by the arrows e400. Once the airflow has passed through the flow conditioning apparatus e200, the turbulence is reduced, as indicated by the aligned arrows e402.

Where the flow conditioning apparatus e200 is provided in the smoking substitute apparatus e150a, e150b, e150c the provision of a bypass passage e180 may reduce the effect of the flow conditioning apparatus e200 on the overall resistance to draw of the smoking substitute apparatus e150a, which may in turn increase the range of possible design parameters for the flow conditioning apparatus e200 allowing air flow past the wick e162 and filament e164 to be more readily optimized for improved aerosol generation without compromising the overall operation of the smoking substitute apparatus e150a, e150b, e150c.

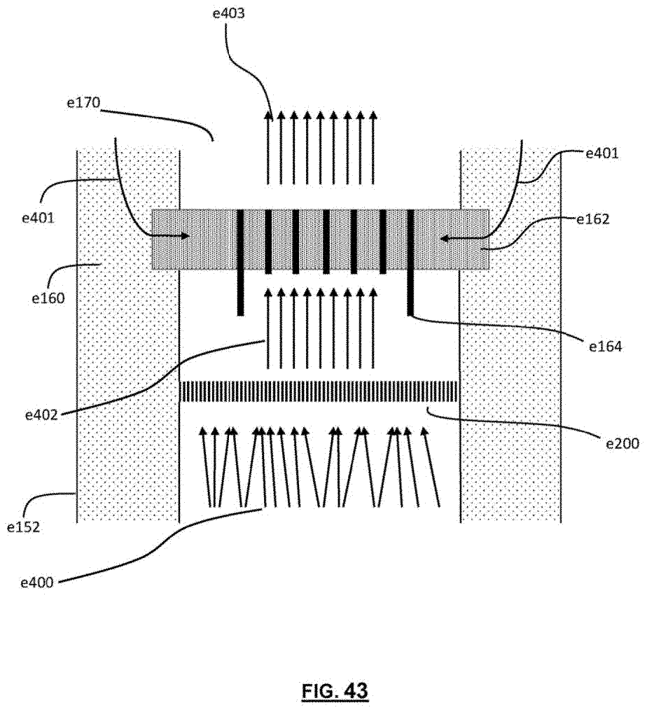

The flow conditioning apparatus e200 may, for example, comprise an air permeable mesh arranged across the vaporizer passage e170 such that air drawn through the vaporizer passage e170 is drawn through the mesh. The mesh may be constructed from an air-impermeable material comprising one or more bores e202 extending through, as illustrated exemplarily in FIG. 44.

The experimental work reported below is relevant to the embodiments disclosed above in view of the effect shown by which the air flow conditions at the wick have an influence on the particle size of the generated aerosol. The provision of a bypass airflow and/or a flow conditioning device affects the air flow conditions at the wick.

Development F

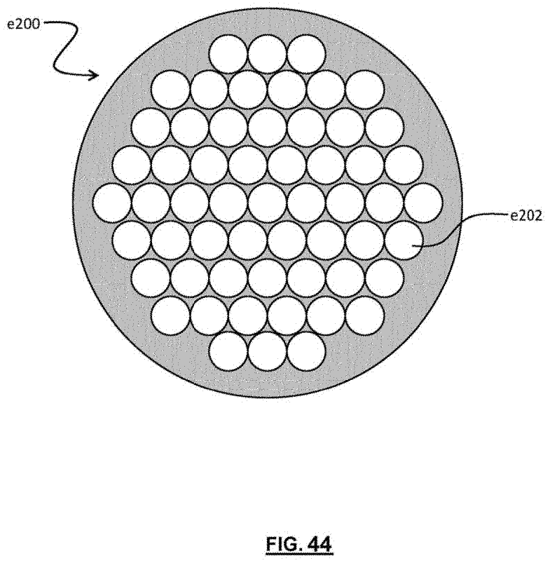

FIG. 45 illustrates a smoking substitute apparatus f250 according to an embodiment of the disclosure. Components and parts of the apparatus that are common to the reference arrangement of FIG. 19 are referenced with the same number (but with an “f” prefix where appropriate), and are not discussed further in view of this embodiment.

The smoking substitute apparatus of the present embodiment differs from that of the reference arrangement in that it includes bypass air flow passages f200 which extend from bypass air inlets f201 to bypass air outlets f202. In FIG. 45, the bypass passages f200 are defined by the casing of the consumable and are located towards the outlet end of the apparatus f250. However, in other embodiments, the bypass passages f200 may be disposed in a different part of the consumable, such as coaxially adjacent to the passage f170 (hereinafter referred to as the “main flow passage”) or through the tank f152.

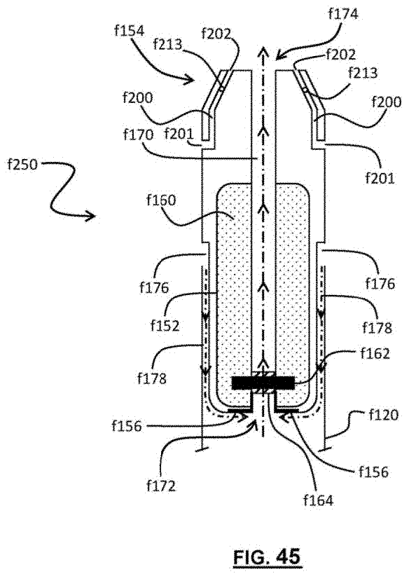

Another difference between the present embodiment and the reference arrangement is the inclusion of flow regulators f213 in the bypass flow passages f200. An exemplary flow regulator f213a is illustrated in FIGS. 46A to 46C. The flow regulator f213a is an annular member made of a resiliently deformable material, providing an aperture with an adjustable size. For example, the regulator f213a may be made from a silicon-based material having a tailored Shore hardness. The range of adjustable sizes of the aperture does not exceed the cross sectional area of the bypass passage f200. Thus, the regulator f213a provides an increased air flow resistance in the bypass passage f200 compared to a corresponding case in which the flow regulator f213a is absent.

FIG. 46A shows the flow regulator f213a disposed in a bypass flow passage f200 in a schematic arrangement, the flow regulator f213a being attached circumferentially to an inner wall of the bypass flow passage f200. When air flows through the bypass passage f200, the aperture is constricted (relaxed) or dilates according to the air pressure difference across the flow regulator f213a. Specifically, when the velocity of air flow through the bypass passage f200 is high, the pressure difference is correspondingly high, and the aperture dilates so as to decrease the resistance to air flow in the bypass passage f200. Similarly, if the velocity of air flow is lowered, the aperture constricts (relaxes) to a small size so as to provide a high resistance to air flow. Accordingly, when a user draws air from the apparatus with a high inhalation flow rate, air velocity through the bypass flow passage f200 is high, and the flow regulator f213a decreases resistance to air flow so as to allow a greater air flow rate in the bypass flow passage f200, thus reducing the proportion of air flow which passes through the main flow passage f170. Similarly, when the inhalation rate is low, the flow regulator provides a high air flow resistance in the bypass passage f200, thus allowing a large proportion of air flow to flow through the main flow passage f170. By varying its size in this way, the flow regulator f213a allows the air velocity of air flowing over the wick f162 to be maintained at a suitable value so as to promote the generation of aerosol droplets having a suitable size distribution. As a numerical example, a suitable size distribution may be characterized by a median droplet size, d₅₀, of at least 1 μm, more preferably d₅₀ is in the range 2-3 μm.

As a further numerical example of air flow rates in the apparatus, when a user inhales with a flow rate of 1 L min⁻¹, 0.6 L min⁻¹ of said airflow may pass through the main passage f170, and 0.4 L min⁻¹ may pass through the bypass passages f200. Whereas when a user inhales with a flow rate of 1.6 L min⁻¹, 0.6 L min⁻¹ (or slightly more than 0.6 L min⁻¹) may pass through the main passage f170 and 1 L min⁻¹ may pass through the bypass passages f200.

FIG. 46B shows a longitudinal cross section of the bypass flow passage f200 and the flow regulator f213a. This view illustrates one suitable mechanism for how the flow regulator f213a dilates and constricts when subjected to a pressure difference thereacross caused by air flowing through the bypass flow passage f200. As the air flows along the passage f200 (illustrated by the dashed arrow), air pushes the free, radially inward portion of the member along the direction of flow. The attached, radially outward portion of the member remains attached to the bypass passage f200 and does not move relative to the passage f200. Thus, the combination of these responses causes the member f213a to resiliently deform, as shown by the dotted lines in FIG. 46B, and therefore dilate the aperture and decrease the resistance to air flow in the passage f200. The cross section of the flow regulator is triangular so that the radially inward portion is thinner than the outward portion and hence the member f213a is more readily deformable at a typical inhalation air flow rate than a corresponding, thicker member. If the rate of air flow is lowered, the radially inward portion moves back towards its original position, so as to constrict the aperture. The member f213a in both constricted and dilated forms is illustrated in FIG. 46C.

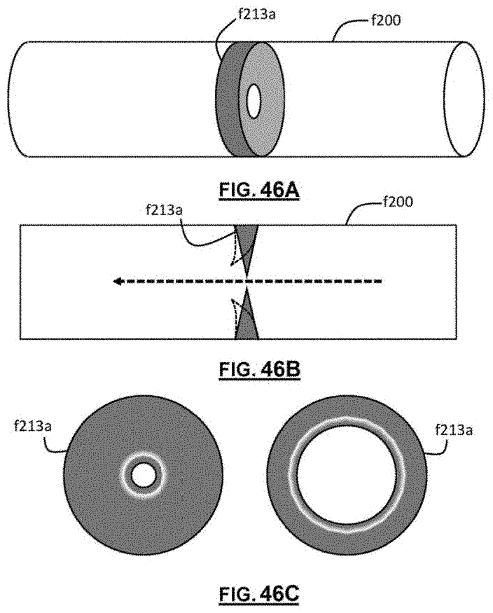

An alternative example of a flow regulator f213b is shown in FIG. 47. The flow regulator f213b is hinged about a point on a wall of the bypass flow passage f200. By rotating about the point as indicated in FIG. 47 (dotted lines), the flow regulator f200 can vary the cross sectional area of air flow through the regulator, which correspondingly varies the resistance to air flow through the bypass flow passage f200. In a similar manner to flow regulator f213a, shown in FIGS. 46A to 46C, the regulator f213b varies resistance to air flow according to the pressure difference thereacross. For example, air flowing through the bypass flow passage f200 exerts a torque on the flow regulator f213b. If sufficiently large, the torque may cause the regulator f213b to hinge about the point so as to increase the cross sectional area of flow through the regulator and decrease resistance to air flow in the passage f200. In the absence of such a torque, i.e., when the flow rate of air through the passage f200 is too low, the flow regulator remains at or returns to a high resistance position (e.g., completely blocking the passage). This may be achieved by a coiled spring attached to the flow regulator f213b which exerts a torque opposite to that caused by air flowing through the passage f200. Alternatively, the regulator may be weighted so as to return to a high resistance position under the force of gravity. Indeed, any means of biasing the regulator to a high resistance position may be used.

Alternative constructions for the flow regulator will be apparent on the basis of the present disclosure. For example, constructions based on slit diaphragms may be used, the slit in the diaphragm opening to an increasing degree with increasing air pressure across it.

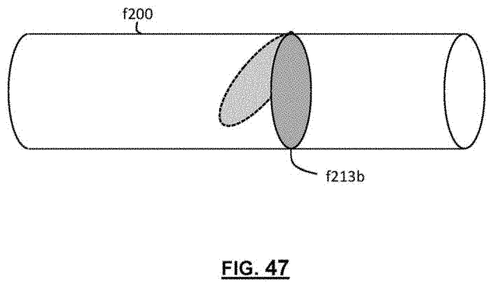

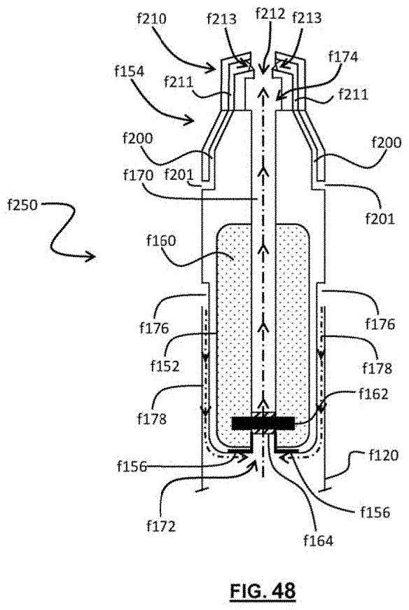

FIG. 48 illustrates the apparatus f250 of FIG. 45 with the inclusion of a mouthpiece f210. The mouthpiece f210 comprises bypass channels f211 and a main channel f212, for engagement with the bypass air flow passages f200 and the main flow passage f170 respectively. The apparatus of FIG. 48 also differs from that of FIG. 45 in that the flow regulators f213 are disposed in the bypass channels f211 of the mouthpiece f210. In this arrangement the mouthpiece is removable from the apparatus f250, though in other embodiments the mouthpiece may be fixed to the apparatus. Having the flow regulators f213 disposed in the mouthpiece f210 allows mouthpieces to be tailored with specific regulators f213 to the requirements of individual users or groups of users. For example, a user with a large inhalation flow rate may choose a mouthpiece with a flow regulator f213 which readily lowers the resistance to flow in the bypass passages f200, and a user with a small inhalation flow rate may choose a mouthpiece with a flow regulator f213 which less readily lowers the resistance to flow in the bypass passages f200. The mouthpiece of FIG. 48, including the flow regulators f213, is illustrated more clearly in enlarged form in FIG. 49.

The experimental work reported below has relevance to the embodiments disclosed above in particular in view of the control provided over the flow conditions at the wick when the flow regulator varies the proportion of air flow through the bypass flow passage compared with air flow through the main flow passage.

Development G

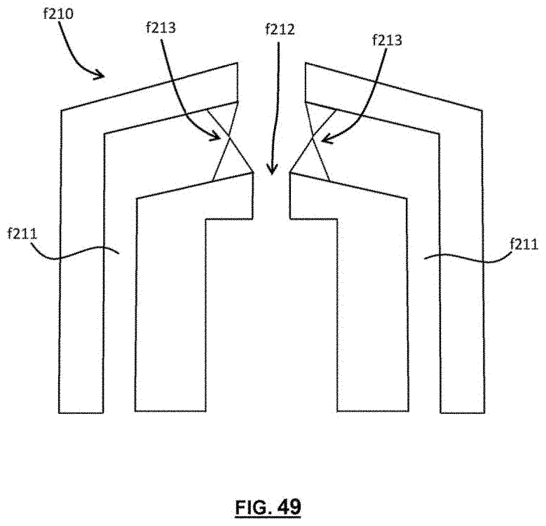

FIG. 50 illustrates a smoking substitute apparatus according to an embodiment of the disclosure. Components and parts of the apparatus that are common to the reference arrangement of FIG. 19 are referenced with the same number (but with a “g” prefix where appropriate), and are not discussed further in view of this embodiment.

The smoking substitute apparatus of the present embodiment differs from that of the reference arrangement in that it includes bypass flow channels g200 which extend from bypass air inlets g201 to the outlet g174. In FIG. 50, the bypass flow channels g200 are defined by the casing of the consumable. However, in other embodiments, the bypass channels g200 may be disposed in a different part of the consumable, such as adjacent to the passage g170 (hereinafter referred to as the “main flow channel”) or through the tank g152.

The present embodiment includes a mouthpiece g210. The mouthpiece g210 comprises bypass passages g211 and a main passage g212, for engagement with the bypass flow channels g200 and the main flow channel g170 respectively. The mouthpiece g210 may be removable from the consumable, or may be fixed to the consumable. Alternatively, in other embodiments, the mouthpiece g210 may be absent.

The effect of the bypass channels g200, and the bypass passages g211 in the mouthpiece g210, is to share an air flow, demanded at the mouthpiece g210, between the main and bypass flow channels g170, g200. Thus, when a user inhales from the mouthpiece g210 at a particular flow rate, the individual flow rates flowing through the bypass channels g200 and main channel g170 are lower than the overall inhalation flow rate.

This effect has the advantage that when a user inhales from the mouthpiece g210 with a flow rate which, in a corresponding case where the bypass channels g200 are absent, would generate an air velocity over the wick g162 that is too high to generate aerosol droplets of a suitable size distribution, the flow rate through the main flow channel g170, and thus the air velocity over the wick g162, remains low enough to generate aerosol droplets having a suitable size distribution. This increases the upper range of inhalation flow rates which can produce an air velocity over the wick g162 that is adequately low for the generation of aerosol droplets having a suitable size distribution. As a numerical example, a suitable size distribution of droplets may be characterized by a median droplet size, d₅₀, of at least 1 μm.

Bypass constrictions g213 are disposed within the bypass flow channels g200. The bypass constrictions g213 are configured to determine the resistance to flow through the bypass flow passage g200 by presenting a smaller cross sectional area than the bypass air inlets g201. In doing so, the bypass constrictions g213 determine the ratio of flow between the bypass flow channels g200 and the main flow channel g170. Therefore, the bypass constrictions g213 can be set according to the inhalation flow rate of a given user or range of users, such that nicotine delivery is consistent for users having different inhalation flow rates.

The bypass constrictions g213 may take one or a combination of the exemplary forms described below. However, the bypass constrictions are not limited to such examples, and could take any form that provides a smaller cross sectional area than that of the bypass air inlet g201.

The bypass constrictions g213 may include tapered regions of the bypass flow channels g200, i.e., the cross sectional area of the bypass flow channel g200 may taper to the smaller cross sectional area. Alternatively, the bypass constrictions g213 may include a portion of uniform cross sectional area, the cross sectional area of the section being equal to the smaller cross sectional area.

The bypass constrictions g213 may include a mesh such as a foraminous mesh which partially blocks the bypass flow channel g200, the combined cross sectional area of the gaps in the mesh being smaller than that of the cross sectional area of one of the bypass air inlets g201.

The bypass constrictions g213 may be configured to generate turbulent air flow along the bypass flow channel g200. Generating turbulent flow inhibits air from flowing through the channel g200, thus such bypass constrictions g213 are capable of providing an additional resistance to air flow in the bypass channels g200 which can be tailored to an individual user or group of users. An example of a bypass constriction which generates turbulent air flow is a constriction including one or more walls having a surface that is rougher than that of the walls of the remainder of the bypass flow channel g200. The rough one or more walls partially break up laminar air flow in the constriction, and force the air into a turbulent flow path.

The bypass constrictions g213 may be configured to adjust to change the resistance to flow along the bypass flow channel g200. When the rate of flow is such that the air velocity over the wick g162 is too high to generate aerosol droplets having a suitable size distribution (e.g., a median droplet size of at least 1 μm), the constrictions g213 may be adjusted to lower the resistance to flow in the bypass flow channel g200, such that the proportion of flow passing through the main flow channel g170 is reduced, thereby lowering the air velocity over the wick g162. Similarly, when the rate of flow is such that the air velocity over the wick g162 is too low, the bypass constrictions g213 may adjust to raise the resistance to flow in the bypass flow channel 200, thereby increasing the air velocity over the wick g162.

Such an adjustable constriction g213 may include a valve. In an open position, the valve provides low resistance to flow through the bypass channel g200 in which it is disposed, thereby minimizing air velocity over the wick g162. In a closed position, the valve partially or completely blocks the respective bypass channel g200 to provide a high (or infinite) resistance to flow, maximizing velocity over the wick g162.

Another such bypass constriction g213 may include two relatively rotatable members. The members may be rotatable between a first position and a second position, these positions differing in terms of the cumulative obstruction presented to airflow. For example, the members may be foraminous meshes. The meshes may reduce flow resistance in the bypass channel g200 in which they are disposed by rotating to align their respective foramina with respect to the axis of the channel g200, or increase flow resistance by misaligning their respective foramina with respect to the axis of the channel g200.

Additionally, the previous examples of non-adjustable constrictions g213 may be adapted to be adjustable. For example, a tapered region may be adjustable such that the tapering angle, the length of the tapered region, or another characteristic of the tapering is variable. For instance, by increasing the tapering angle such that the tapered region tapers to a narrower cross-sectional area, the resistance to flow in the respective bypass channel g200 increases. Conversely, the flow resistance may be reducible by lowering the tapering angle.

In another example, the bypass constriction g213 having a uniform smaller cross sectional area may be narrowable or widenable so as to adjust the resistance to flow.

Also, the single mesh may be configured to adjust the resistance to flow in the respective bypass channel g200 by sliding in and out of the channel g200.

A way of performing such adjustments, in particular for the constrictions having either the tapered portion, rough portion, uniform cross sectional area portion, or a combination thereof, is to form the constriction walls of a flexible material, and press a suitably shaped die into the material to constrict the bypass flow channel g200.

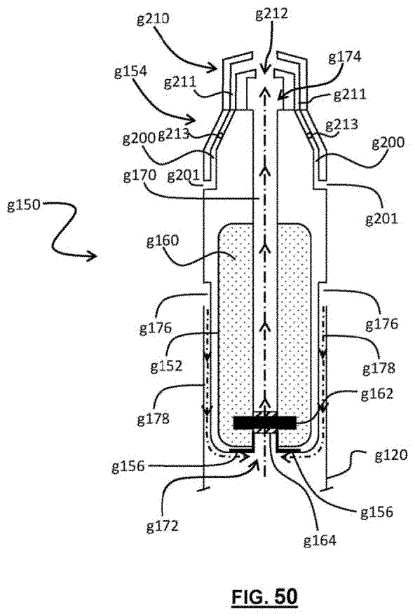

FIG. 51 illustrates a smoking substitute apparatus according to a further embodiment of the disclosure. Components and parts of the apparatus that are common to the reference arrangement of FIG. 19 are referenced with the same number (but with a “g” prefix where appropriate), and are not discussed further in view of this embodiment. Furthermore, components and parts of the apparatus that are common to the embodiment of FIG. 50 are referenced with the same number.

A main constriction g214 and bypass constrictions g213 are disposed within the main and bypass flow channels g170, g200 respectively. Each constriction g214, g213 is configured to determine the resistance to flow through the flow channel g170, g200, in which it is disposed, by presenting a smaller cross sectional area than the air inlet of the channel in which it is disposed g172, g201. In doing so, the constrictions g214, g213 determine the ratio of flow between the bypass flow channels g200 and the main flow channel g170. Therefore, the constrictions g214, g213 can be set according to the inhalation flow rate of a given user or range of users, such that nicotine delivery is consistent for users having different inhalation flow rates. Additionally, as the constrictions g214, g213 are disposed along both the main flow channel g170 and the bypass flow channel g200, the overall flow rate out of the one or more outlets can be controlled, i.e. the constrictions g214, g213 may be configured to restrict the rate of flow with which a user can inhale from the mouthpiece g210. This has the advantage that, for users with high inhalation flow rates, the air inhaled by the user is not diluted with a large proportion of bypass air flow containing no aerosol.

Any one or combination of constrictions g214, g213 may take one or a combination of the exemplary forms described below. However, the constrictions g214, g213 are not limited to such examples, and could take any form that provides a smaller cross sectional area than that of the air inlet g172, g201 in which each respective constriction g214, g213 is disposed.

Any one or combination of the constrictions g214, g213 may include tapered regions of the flow channel g170, g200 in which the constriction is disposed, i.e. the cross sectional area of the flow channel g170, g200 may taper to a cross sectional area smaller than that of the air inlet g172, g201 of the channel g170, g200 in which the constriction g214, g213 is disposed. Alternatively, any one or combination of the constrictions g214, g213 may include a portion of uniform cross sectional area, the portion having a smaller cross sectional area than that of the air inlet g172, g201 of the channel g170, g200 in which the constriction g214, g213 is disposed.

Any one or combination of the constrictions g214, g213 may include a mesh such as a foraminous mesh which partially blocks the flow channel g170, g200 in which the constriction g214, g213 is disposed, the combined cross sectional area of the gaps in the mesh being smaller than that of the cross sectional area of the air inlet g172, g201 of the channel g170, g200 in which the constriction g214, g213 is disposed.

Any one or combination of the constrictions g214, g213 may be configured to generate turbulent air flow along the flow channel g170, g200 in which the constriction g214, g213 is disposed. Generating turbulent flow inhibits air from flowing through the channel g170, g200, thus such constrictions are capable of providing an additional resistance to air flow in the channel g170, g200 which can be tailored to an individual user or group of users. An example of a constriction which generates turbulent air flow is a constriction including one or more walls having a surface that is rougher than that of the walls of the remainder of the channel g170, g200 in which the constriction g214, g213 is disposed. The rough one or more walls partially break up laminar air flow through the constriction, and force the air into a turbulent flow path.

Any one or combination of the constrictions g214, g213 may be configured to adjust to change the resistance to flow along the flow channel g170, g200 in which the constriction g214, g213 is disposed. When the rate of flow is such that the air velocity over the wick g162 is too high to generate aerosol droplets having a suitable size distribution (e.g., a median droplet size of at least 1 μm, more preferably in the range 2-3 μm), the constrictions g214, g213 may be adjusted to alter the respective resistances to flow in the flow channels g170, g200, such that the proportion of flow passing through the main flow channel g170 is reduced, thereby lowering the air velocity over the wick g162. Similarly, when the rate of flow is such that the air velocity over the wick g162 is too low, the constrictions g214, g213 may adjust to change the respective resistances to flow in the flow channels g170, g200, thereby increasing the air velocity over the wick g162.

Such an adjustable constriction g214, g213 may include a valve. In an open position, the valve provides low resistance to flow through the channel g170, g200 in which it is disposed. In a closed position, the valve partially or completely blocks the flow channel g170, g200 in which it is disposed to provide a high (or infinite) resistance to flow, maximizing velocity over the wick g162.

Another such constriction g214, g213 may include two relatively rotatable members. The members may be rotatable between a first position and a second position, these positions differing in terms of the cumulative obstruction presented to airflow. For example, the members may be foraminous meshes. The meshes may reduce air flow resistance in the flow channel g170, g200 in which they are disposed by rotating to align their respective foramina with respect to the axis of the channel g170, g200, or increase air flow resistance by misaligning their respective foramina with respect to the axis of the channel g170, g200.

Additionally, the previous examples of non-adjustable constrictions g214, g213 may be adapted to be adjustable. For example, a tapered region may be adjustable such that the tapering angle, the length of the tapered region, or another characteristic of the tapering is variable. For instance, by increasing the tapering angle such that the tapered region tapers to a narrower cross-sectional area, the resistance to flow in the respective channel g170, g200 increases. Conversely, the flow resistance may be reducible by lowering the tapering angle.

In another example, the constriction g214, g213 having a uniform cross sectional area may be narrowable or widenable so as to adjust the resistance to air flow.

Also, the single mesh may be configured to adjust the resistance to flow in the respective channel g170, g200 by sliding in and out of the channel g170, g200.

A way of performing such adjustments, in particular for the constrictions having either the tapered portion, rough portion, uniform cross sectional area portion, or a combination thereof, is to form the constriction walls of a flexible material, and press a suitably shaped die into the material to constrict the respective flow channel g170, g200.

The resistance to flow along the bypass channel caused by the bypass constriction and optionally the resistance to flow along the main flow channel caused by the main constriction determine the ratio of flow between the bypass channel and the main flow channel. In the exemplary embodiments, the ratio of flow is configured such that, when an average 1.3 Lpm is drawn through the device, the ratio splits it in such a way that 0.5 Liters per minute (Lpm) travels through the vaporization chamber (e.g., along the main flow channel) and 0.8 Lpm travels down the bypass airflow channel. That is the bypass constriction and optionally the main constriction are configured to generate resistance along the bypass channel and main flow channel such that when an average 1.3 Lpm is drawn through the device, the ratio splits the airflow in such a way that 0.5 Lpm travels along the main flow channel. The ratio may be around 5:8 or between 1:2 and 3:4. That is the ratio may split the airflow so that when 1.3 Lpm is drawn through one or more outlets of a smoking substitute device, 5 parts of the airflow is caused to be drawn through the main channel and 8 parts of the airflow is caused to be drawn through the bypass channel. With the vaporization chamber (e.g., a wick and heater) being arranged in the main flow channel, the ratio causes a reduced airflow in the vaporization chamber that generates advantageous particle sizes.

The experimental work reported below is relevant to the embodiments disclosed above in view of the configurability of the apparatus to change the resistance to flow along the main channel relative to the bypass channel. This affects the flow conditions at the wick and, as the following disclosure shows, therefore affects the particle size of the generated aerosol.

Development H

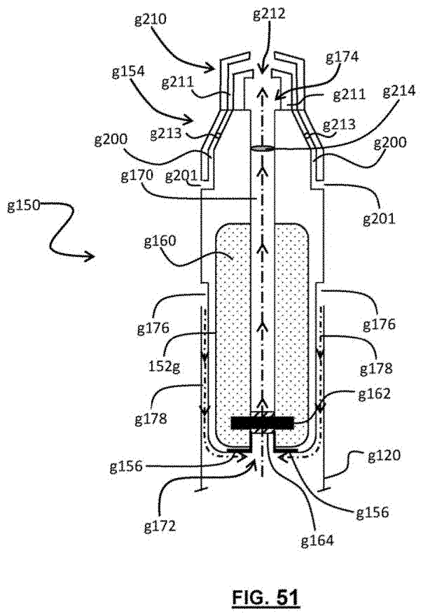

FIG. 52 illustrates a schematic longitudinal cross sectional view of a first embodiment of the smoking substitute apparatus forming part of the smoking substitute system shown in FIGS. 17 and 18. The embodiment illustrated in FIG. 52 differs from the reference arrangement illustrated in FIG. 19 in that the substitute smoking apparatus includes two bypass passages h180 in addition to the vaporizer passage h170. The bypass passages correspond to the second air passage of the claims. In other embodiments, the number of bypass passages may be greater or smaller than in the illustrated example. In the illustrated embodiment, the bypass inlets h182 are separate from the vaporizer air inlet h172. In other embodiments, an inlet may be connected to both the vaporizer air passage h170 and the bypass air passage h180. Furthermore, in the illustrated embodiment, the bypass air outlets h184 open onto the vaporizer air passage h170. In other embodiments, the bypass air outlets h184 may open from the housing of the smoking substitute apparatus h150a.

The provision of a bypass passage h180 means that a part of the air drawn through the smoking substitute apparatus h150a when a user inhales via the mouthpiece h154 is not drawn through the vaporization chamber. This has the effect of reducing the flow rate through the vaporization chamber in correspondence with the respective flow resistances presented by the vaporizer passage h170 and the bypass passage h180. This can reduce the correlation between the flow rate through the smoking substitute apparatus h150 (i.e., the user's draw rate) and the particle size generated when the e-liquid h160 is vaporized and subsequently forms an aerosol. Therefore, the smoking substitute apparatus h150 of the present embodiment can deliver a more consistent aerosol to a user.

The mouthpiece h154a of the smoking substitute apparatus h150a comprises a part of the bypass air channel h180 and a part of the vaporizer air channel h170. The mouthpiece h154a is shaped so as to create a flow constriction in the part of the bypass air channel h180 that is comprised within the mouthpiece h154a. In the illustrated embodiment, a component h190 of the mouthpiece h154a forms a part of the wall of the bypass passage h180. The component h190 includes a projection h192 which narrows the bypass passage h180 so as to restrict the airflow through it. In other embodiments (not illustrated) the component h190 may not comprise any projections into the passage h180, but may instead be shaped so as to constrict the entire passage. The level of constriction provided to the passage h180 by the component h190 may be selected so as to control the ratio between the air flow rate in the bypass passage h180 and the air flow rate in the vaporizer passage h170.

The optimal ratio between the air flow rate in the bypass passage h180 and the air flow rate in the vaporizer passage h170 may be determined from representative user inhalation rates and from the required air flow rate through the vaporization chamber to deliver a desired droplet size. For example, an average total flow rate of 1.3 liters per minute (during inhalation by the user) may be split such that 0.8 liters per minute passes through the bypass air channel h180, and 0.5 liters per minute passes through the vaporizer channel h170, a bypass:vaporizer flow rate ratio of 1.6:1. Such a flow rate may provide a droplet size, d₅₀, of 1-3 μm (more preferably 2-3 μm) with a span of not more than 20 (preferably not more than 10). Alternative flow rate ratios may be provided based on calculations and measurements of user flow rate, vaporizer flow rate, and droplet size d₅₀. A bypass:vaporizer flow rate ratio of between 0.5:1 and 20:1, typically at an average total flow rate of 1.3 liters per minute may be advantageous depending on the configuration of the smoking substitute apparatus.

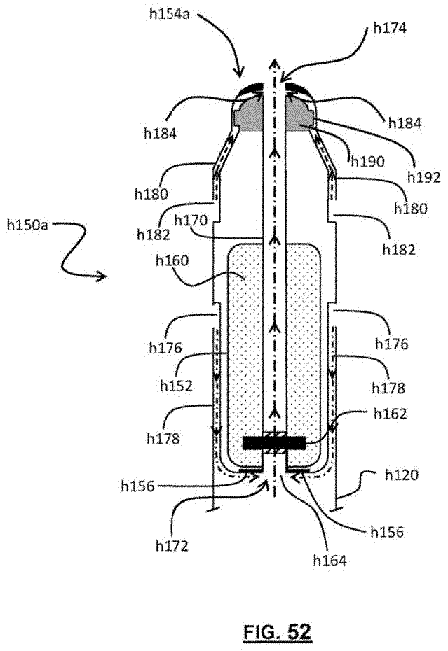

FIG. 53 illustrates a smoking substitute apparatus h150b according to the second embodiment. The smoking substitute apparatus h150b according to the second embodiment differs from the smoking substitute apparatus h150a of the first embodiment in that the mouthpiece h154b is a removably engageable component of the smoking substitute apparatus h150b. Such an arrangement may allow a user to select a mouthpiece h154b with a different level of bypass channel constriction according to their typical inhalation rate. For example, a user with a high inhalation rate may select a mouthpiece h154b with less constricted bypass channels h180. This would, in turn, allow a higher flow rate through the bypass channels h180 so as to maintain the desired flow rate through the vaporizer channel h170 and/or vaporization chamber. In this embodiment, the constricting component h190b is an integral component of the mouthpiece h154b, and the mouthpiece h154b must therefore be replaced if the user wishes to utilize a different level of constriction.

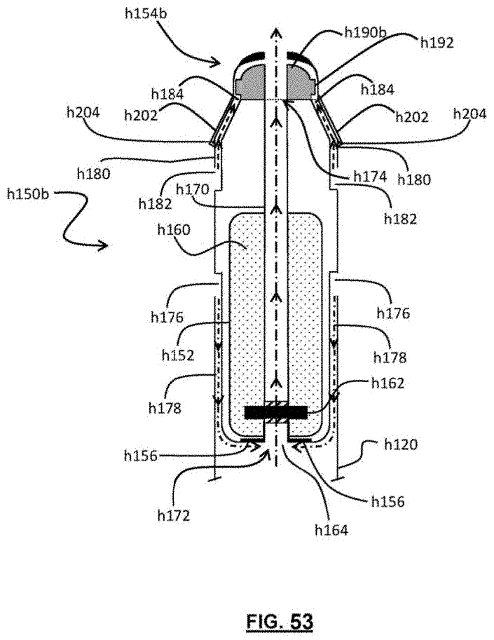

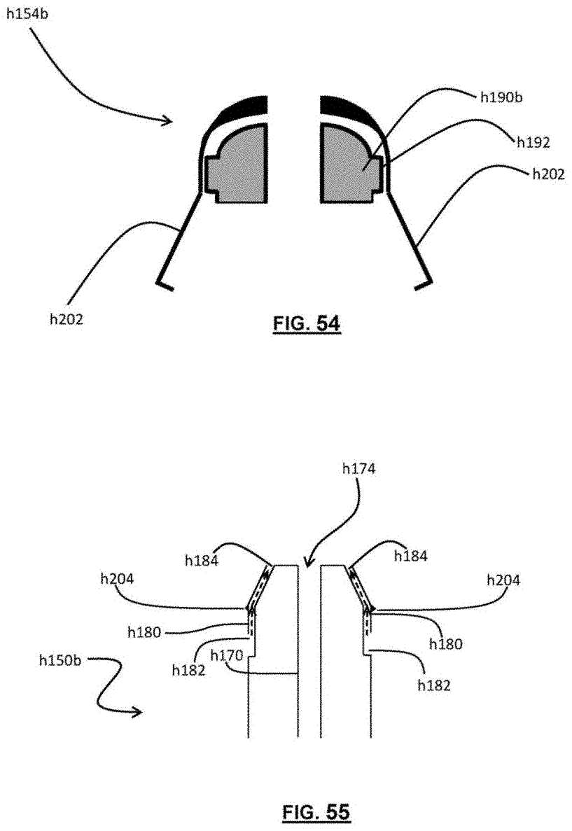

The mouthpiece h154b comprises positioning elements h202, which are configured to engage with the housing of the smoking substitute apparatus h150b. In the illustrated embodiment, the smoking substitute apparatus comprises fixing projections h204, which form part of a snap fit engagement with the positioning elements h202 of the mouthpiece h154b. These fixing projections h204 and positioning elements h202 are further illustrated in FIGS. 54 and 55, which show an enlarged view of the mouthpiece h154b (FIG. 23) and of the smoking substitute apparatus h150b with the mouthpiece removed (FIG. 55). In other embodiments, alternative means of engagement between the mouthpiece h154b and the smoking substitute apparatus h150b may be provided. For example, the mouthpiece h154b may be fitted via a screw fit engagement, or via an interference fit.

A mouthpiece h154b may be provided in a retail pack comprising one or more mouthpieces h154b. The pack may comprise mouthpieces h154b each having the same level of constriction from the respective constricting component h190b. Alternatively, the pack may comprise mouthpieces h154b each having a different level of constriction from the respective constricting component h190b to provide a selection of possible constriction levels to the user.

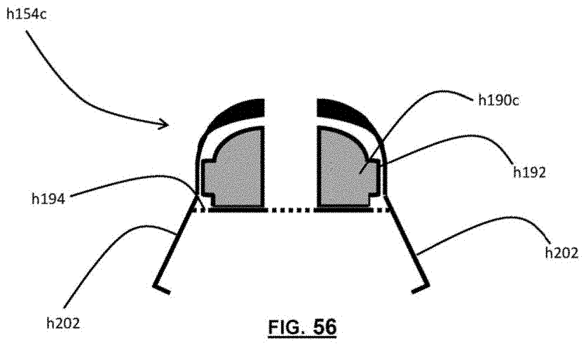

In a third embodiment, as illustrated in FIG. 56, the constricting component h190c is a replaceable component of the mouthpiece h154c. As with the mouthpiece h154b of the second embodiment, the mouthpiece according to the third embodiment may permit a user to change the level of constriction of the bypass channel h180 in order to maintain a suitable flow rate through the vaporizer channel h170. In this case, only the constricting component h190c needs to be replaced to effect this change, and therefore the number of parts which must be replaced is reduced. Further, in a mouthpiece h154c according to the third embodiment, the constricting component h190c may be removed from the mouthpiece to facilitate cleaning of the mouthpiece h154c and the constricting component h190c. The mouthpiece h154c may comprise positioning components h194 to hold the constricting component h190c in place within the mouthpiece h154c. For example, a plate h194 may be fitted in the mouthpiece via a screw or interference fit. In alternative embodiments, a clip or other fastener may be provided. Alternatively, the constricting component h190c may be held in place between the interior of the mouthpiece h154c and the end of the smoking substitute apparatus h150b when the two parts are engaged. The constricted air passages within the mouthpiece h154c may be fully defined by the constricting component h190c. Alternatively, the constricted air passages may be defined by a space between the constricting component h190c and the interior of the mouthpiece h154c.

The constricting component h190c in this third embodiment may be constructed from an elastomeric material such as silicone. Alternatively, the constricting component h190c in this third embodiment may be constructed from a rigid material such as a plastic. The constricting component h190c may comprise features to allow the level of constriction to be identified. For example, a constricting component h190c with a particular level of constriction may be formed of a material of one color, while a constricting component h190c with a different level of constriction may be formed of a material of second color.

A constricting component h190c may be provided in a retail pack comprising one or more constricting components h190c. The pack may comprise constricting components h190c each having the same level of constriction. Alternatively, the pack may comprise constricting components h190c each having a different level of constriction to provide a selection of possible constriction levels to the user.

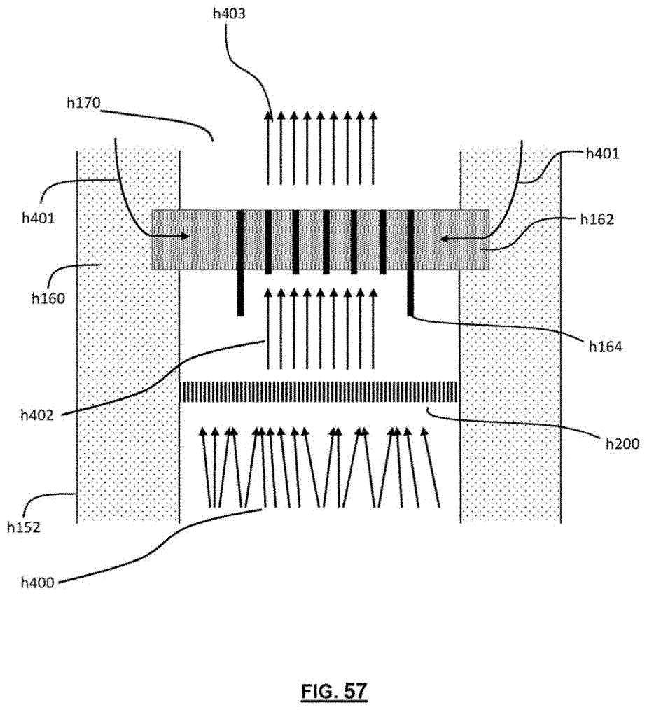

The smoking substitute apparatus may further comprise a flow conditioning apparatus h210 arranged upstream of the wick h162 and filament h164 in the vaporizer passage h170, as illustrated in FIG. 57. The flow conditioning apparatus h210 is configured to reduce turbulence in the air flow through the vaporization chamber and past the wick h162 and filament h164. This may be advantageous for improving aerosol generation, and for reducing the spread in the aerosol particle size distribution. In FIG. 57, the turbulent air flow is indicated by the arrows h400. Once the airflow has passed through the flow conditioning apparatus h210, the turbulence is reduced, as indicated by the aligned arrows h402.

Where the flow conditioning apparatus h210 is provided in the smoking substitute apparatus h150a, h150b, h150c the provision of a bypass passage h180 may reduce the effect of the flow conditioning apparatus h210 on the overall resistance to draw of the smoking substitute apparatus h150a, which may in turn increase the range of possible design parameters for the flow conditioning apparatus h210. This can, in turn, allow air flow past the wick h162 and filament h164 to be more readily optimized for improved aerosol generation without compromising the overall operation of the smoking substitute apparatus h150a, h150b, h150c. The provision of a bypass passage h180 with a constricting component h190a, h190b, h190c may allow airflow through the flow-conditioning apparatus past the wick h162 and filament h164 to be maintained in a narrower range across users with different draw rates, further ensuring consistency of operation of the smoking substitute apparatus h150a, h150b, h150c.

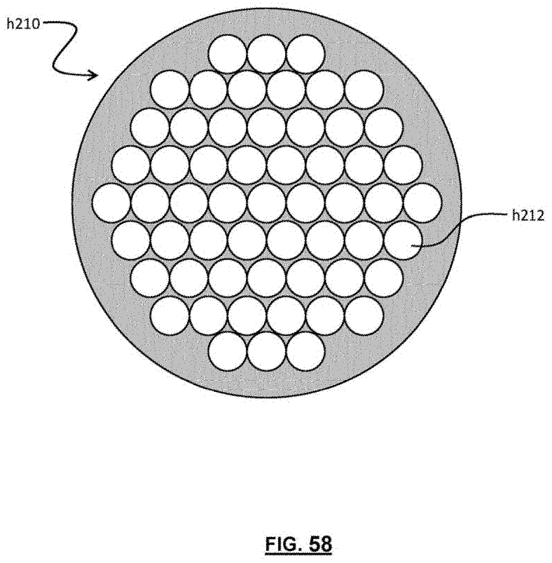

The flow conditioning apparatus h210 may, for example, comprise an air permeable mesh arranged across the vaporizer passage h170 such that air drawn through the vaporizer passage h170 is drawn through the mesh. The mesh may be constructed from an air-impermeable material comprising one or more bores h212 extending through, as illustrated exemplarily in FIG. 58. The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the disclosure in diverse forms thereof.

The experimental work reported below has relevance to the embodiments described above in particular in view of the effect of the conditions at the wick brought about by the control of the bypass air flow in the embodiments. Such control affects the velocity of air flow close to the wick and also affect the cooling rate experienced by vapor emitted from the wick.

Development I

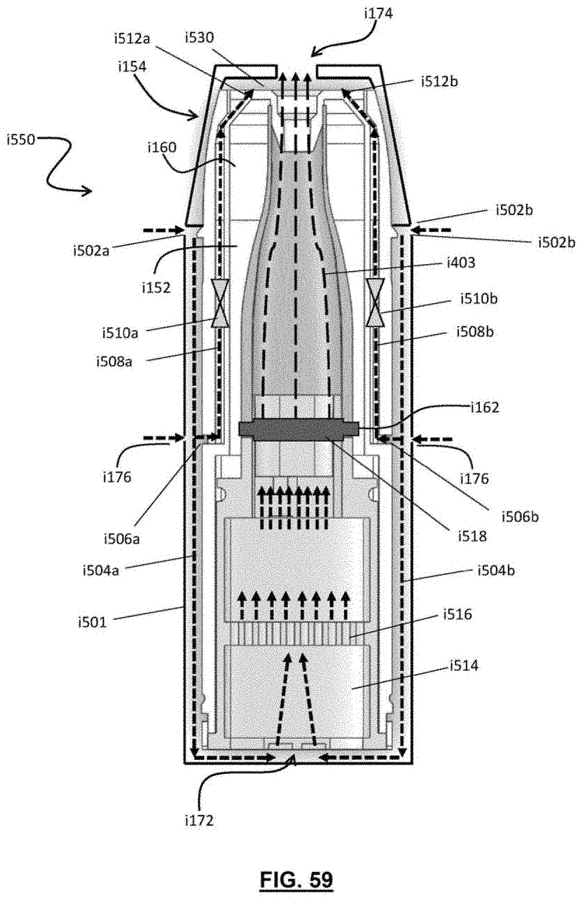

FIG. 59 shows a schematic longitudinal cross sectional view of a smoking substitute apparatus i550 according to the first embodiment. It shares a number of features with the reference arrangement shown in FIG. 19, and so like features are indicated by like reference numerals (but with an “I” prefix where appropriate). The smoking substitute apparatus has a housing i501, within which the majority of its components are contained. Holes within the housing provide the primary air inlets i176 and secondary air inlets i502a/i502b. In this example, the secondary air inlets i502a/i502b, located on either side of the smoking substitute apparatus, are closer to the mouthpiece i154 than the primary air inlets i176.

The primary air inlets i176 and secondary air inlets i502a/i502b are fluidly connected to shared airflow paths i504a/i504b. These shared airflow paths extend along an inside of the smoking substitute apparatus i550 to the vaporization chamber inlet i172. The shared airflow paths are an example of the primary airflow path referred to previously. Also fluidly connected to the primary air inlets i176 and secondary air inlets i502a/i502b are bypass air inlets i506a and i506b (one on either side of the smoking substitute apparatus). The bypass air inlets connect to bypass air ducts i508a and i508b respectively. Each of the bypass air duct contains an adjustable airflow restrictor i510a/i510b. These adjustable airflow restrictors allow a user to tune or adjust the draw resistance of the smoking substitute apparatus in a manner which does not (at least directly) affect the airflow through the vaporization chamber inlet i172. The bypass airflow ducts i508a/i508b end at bypass air duct outlets i512a/i512b which are located proximal to the smoking substitute apparatus outlet i174.

Also shown in FIG. 59 are further features of the vaporization chamber located downstream of the vaporizer chamber inlet i172. After passing through the inlet, the airflow expands into plenum i514. The plenum functions to slow the velocity of the air which is flowing towards the vaporizer. Between the plenum and vaporizer i518, which is a coil and wick arrangement, is a flow straightener i516. The flow straightener in this example is formed from an array of tubes having a cylindrical axis aligned with a flow direction towards the coil and wick arrangement. The flow straightener can also be provided as a mesh.

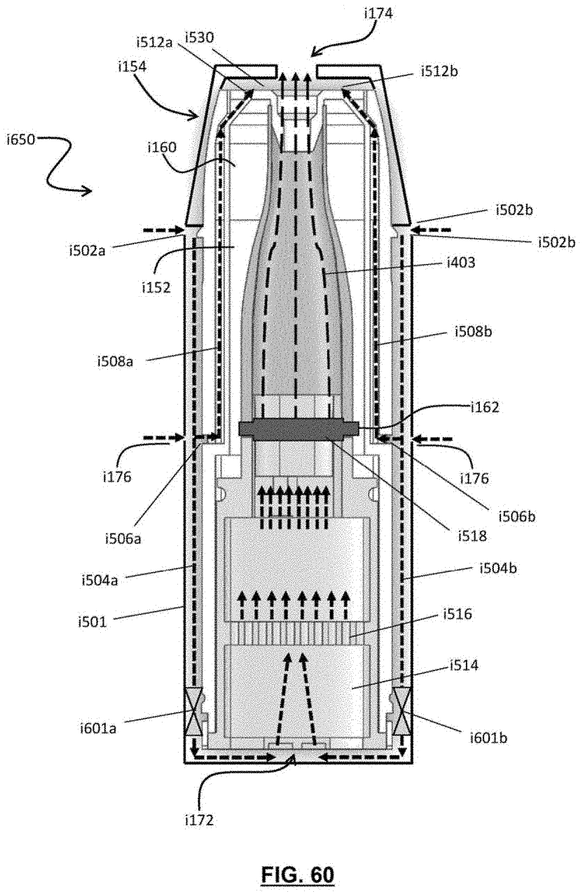

FIG. 60 shows a smoking substitute apparatus i600 according to second embodiment. It shares a number of features with the first embodiment, and so like features are indicated by like reference numerals. In contrast to FIG. 59, the adjustable flow restrictors i601a and i601b are located within the shared airflow paths i504a and i504b. This allows the user to tune or adjust the draw resistance of the smoking substitute apparatus which also influences the air flow properties over and around the vaporizer. For example, by decreasing the draw resistance, the user may be able to draw air through the vaporizer and a higher velocity which may affect the particle size distribution of the aerosol generated therefrom.

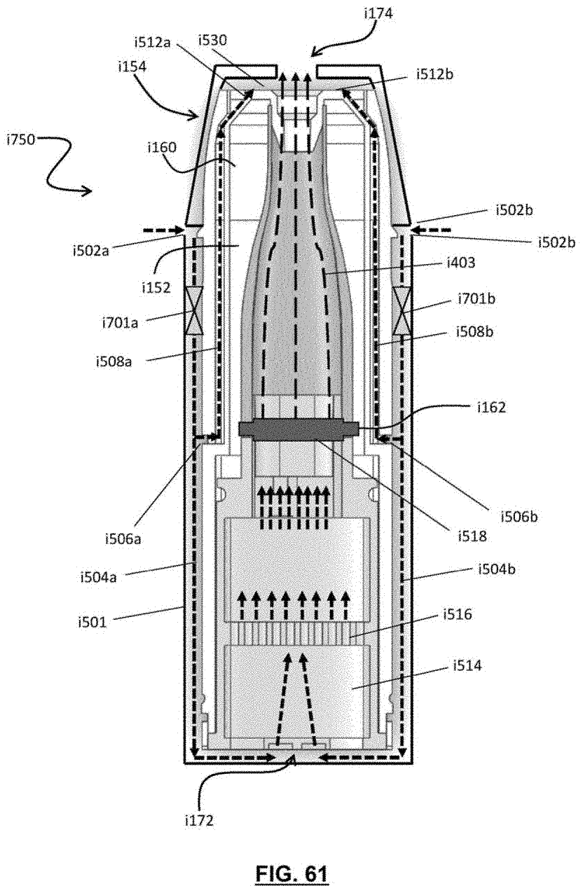

FIG. 61 shows a smoking substitute apparatus i750 according to a third embodiment. It shares a number of features with the first and second embodiments, and so like features are indicated by like reference numerals. In contrast to FIGS. 59 and 60, the smoking substitute apparatus i750 of FIG. 61 has only one air inlet i502a/i502b located in an outer housing of the smoking substitute apparatus. Therefore, the shared airflow path extends from the air inlet i502a/i502b to the bypass airflow inlet i506a/i506b, before separating. In this example then, bypass airflow inlet(s) i506a/i506b provide the second air inlet(s) into the secondary airflow path. A further difference is that the adjustable airflow restrictor i701a/i701b is located in the shared airflow path located between the air inlet i502a/i502b and the bypass airflow inlet. This allows the user to tune or adjust the draw resistance of the smoking substitute apparatus in a manner which affects the airflow through both the bypass airflow ducts i508a/i508b and the vaporizer chamber.

### EXAMPLES

There now follows a disclosure of certain examples of experimental work undertaken to determine the effects of certain conditions in the smoking substitute apparatus on the particle size of the generated aerosol. However, the present disclosure is to be understood to not be limited in its application to the specific experimentation, results, and laboratory procedures disclosed herein after. Rather, the Examples are simply provided as one of various embodiments and are meant to be exemplary, not exhaustive.

The experimental work reported below, to determine the effects of certain conditions in the smoking substitute apparatus on the particle size of the generated aerosol, is relevant in particular in view of the approach taken by the embodiments of the disclosure to control the flow conditions at the wick.

Introduction

Aerosol droplet size is a considered to be an important characteristic for smoking substitution devices. Droplets in the range of 2-5 μm are preferred in order to achieve improved nicotine delivery efficiency and to minimize the hazard of second-hand smoking. However, at the time of writing (September 2019), commercial EVP devices typically deliver aerosols with droplet size averaged around 0.5 μm, and to the knowledge of the inventors not a single commercially available device can deliver an aerosol with an average particle size exceeding 1 μm.

The present inventors speculate, without themselves wishing to be bound by theory, that there has to date been a lack of understanding in the mechanisms of e-liquid evaporation, nucleation and droplet growth in the context of aerosol generation in smoking substitute devices. The present inventors have therefore studied these issues in order to provide insight into mechanisms for the generation of aerosols with larger particles. The present inventors have carried out experimental and modelling work alongside theoretical investigations, leading to significant achievements as now reported.

This disclosure considers the roles of air velocity, air turbulence and vapor cooling rate in affecting aerosol particle size.

Experiments

In the following examples, a Malvern PANalytical Spraytec laser diffraction system was employed for the particle size measurement. In order to limit the number of variables, the same coil and wick (1.5 ohms Ni—Cr coil, 1.8 mm Y07 cotton wick), the same e-liquid (1.6% freebase nicotine, 65:35 propylene glycol (PG)/vegetable glycerin (VG) ratio, no added flavor) and the same input power (10 W) were used in all experiments. Y07 represents the grade of cotton wick, meaning that the cotton has a linear density of 0.7 grams per meter.

Particle sizes were measured in accordance with ISO 13320:2009(E), which is an international standard on laser diffraction methods for particle size analysis. This is particularly well suited to aerosols, because there is an assumption in this standard that the particles are spherical (which is a good assumption for liquid-based aerosols). The standard is stated to be suitable for particle sizes in the range 0.1 micron to 3 mm.

The results presented here concentrate on the volume-based median particle size Dv50. This is to be taken to be the same as the parameter d₅₀ used above.

### First Example: Rectangular Tube Testing

The work of a first example reported here based on the inventors' insight that aerosol particle size might be related to: 1) air velocity; 2) flow rate; and 3) Reynolds number. In a given EVP device, these three parameters are inter-linked to each other, making it difficult to draw conclusions on the roles of each individual factor. In order to decouple these factors, experiments of a first example were carried out using a set of rectangular tubes having different dimensions. These were manufactured by 3D printing. The rectangular tubes were 3D printed in an MJP 2500 3D printer. FIG. 1 illustrates the set of rectangular tubes. Each tube has the same depth and length but different width. Each tube has an integral end plate in order to provide a seal against air flow outside the tube. Each tube also has holes formed in opposing side walls in order to accommodate a wick.

FIG. 2 shows a schematic perspective longitudinal cross sectional view of an example rectangular tube 1170 with a wick 1162 and heater coil 1164 installed. The location of the wick is about half way along the length of the tube. This is intended to allow the flow of air along the tube to settle before reaching the wick.

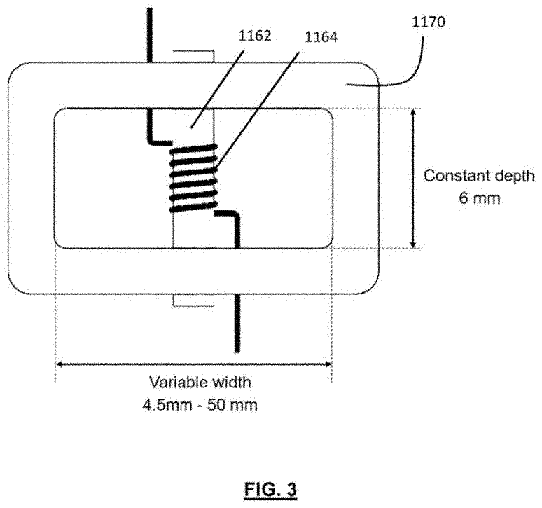

FIG. 3 shows a schematic transverse cross sectional view an example rectangular tube 1170 with a wick 1162 and heater coil 1164 installed. In this example, the internal width of the tube is 12 mm.

The rectangular tubes were manufactured to have same internal depth of 6 mm in order to accommodate the standardized coil and wick, however the tube internal width varied from 4.5 mm to 50 mm. In this disclosure, the “tube size” is referred to as the internal width of rectangular tubes.

The rectangular tubes with different dimensions were used to generate aerosols that were tested for particle size in a Malvern PANalytical Spraytec laser diffraction system. An external digital power supply was dialed to 2.6 A constant current to supply 10 W power to the heater coil in all experiments. Between two runs, the wick was saturated manually by applying one drop of e-liquid on each side of the wick.

Three groups of experiments were carried out in this study of a first example:

1.3 lpm (liters per minute, L min⁻¹ or LPM) constant flow rate on different size tubes

2.0 lpm constant flow rate on different size tubes

1 m/s constant air velocity on 3 tubes: i) 5 mm tube at 1.4 lpm flow rate; ii) 8 mm tube at 2.8 lpm flow rate; and iii) 20 mm tube at 8.6 lpm flow rate.

Table 1 shows a list of experiments of a first example. The values in “calculated air velocity” column were obtained by simply dividing the flow rate by the intersection area at the center plane of wick. Reynolds numbers (Re) were calculated through the following equation:

$$Re = \frac{ρ v L}{μ} ,$$

where: ρ is the density of air (1.225 kg/m³); v is the calculated air velocity in table 1; μ is the viscosity of air (1.48×10⁻⁵ m²/s); L is the characteristic length calculated by:

$$L = \frac{4 P}{A} ,$$

where: P is the perimeter of the flow path's intersection, and A is the area of the flow path's intersection.

TABLE 1
List of experiments in the rectangular tube study

| | | | | Calculated air |
|---|---|---|---|---|
| | Tube size | Flow rate | Reynolds | velocity |
| | [mm] | [lpm] | number | [m/s] |
| 1.3 lpm | 4.5 | 1.3 | 153 | 1.17 |
| constant flow | 6 | 1.3 | 142 | 0.71 |
| rate | 7 | 1.3 | 136 | 0.56 |
| | 8 | 1.3 | 130 | 0.47 |
| | 10 | 1.3 | 120 | 0.35 |
| | 12 | 1.3 | 111 | 0.28 |
| | 20 | 1.3 | 86 | 0.15 |
| | 50 | 1.3 | 47 | 0.06 |
| 2.0 lpm | 4.5 | 2.0 | 236 | 1.81 |
| constant flow | 5 | 2.0 | 230 | 1.48 |
| rate | 6 | 2.0 | 219 | 1.09 |
| | 8 | 2.0 | 200 | 0.72 |
| | 12 | 2.0 | 171 | 0.42 |
| | 20 | 2.0 | 132 | 0.23 |
| | 50 | 2.0 | 72 | 0.09 |
| 1.0 m/s | 5.0 | 1.4 | 155 | 1.00 |
| constant air | 8 | 2.8 | 279 | 1.00 |
| velocity | 20 | 8.6 | 566 | 1.00 |

Five repetition runs were carried out for each tube size and flow rate combination. Between adjacent runs there were at least 5 minutes wait time for the Spraytec system to be purged. In each run, real time particle size distributions were measured in the Spraytec laser diffraction system at a sampling rate of 2500 per second, the volume distribution median (Dv50) was averaged over a puff duration of 4 seconds. Measurement results were averaged and the standard deviations were calculated to indicate errors as shown in section 4 below.

### Second Example: Turbulence Tube Testing

The Reynolds numbers in Table 1 are all well below 1000, therefore, it is considered fair to assume all the experiments of a first example would be under conditions of laminar flow. Further experiments (of a second example) were carried out and reported in this section to investigate the role of turbulence.

Turbulence intensity was introduced as a quantitative parameter to assess the level of turbulence. The definition and simulation of turbulence intensity is discussed below.

Different device designs were considered in order to introduce turbulence. In the experiments of the second example reported here, jetting panels were added in the existing 12 mm rectangular tubes upstream of the wick. This approach enables direct comparison between different devices as they all have highly similar geometry, with turbulence intensity being the only variable.

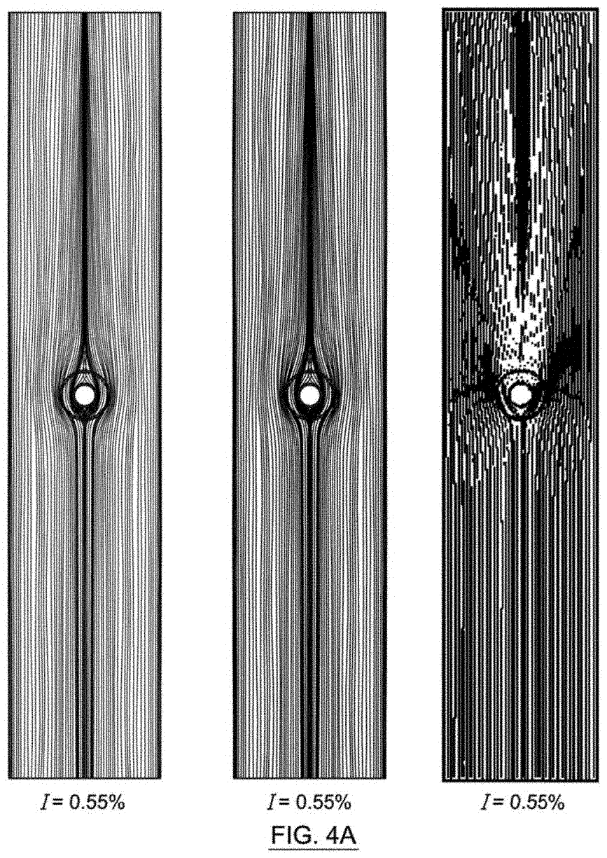

FIGS. 4A-4D show air flow streamlines in the four devices used in this turbulence study of the second example. FIG. 4A is a standard 12 mm rectangular tube with wick and coil installed as explained previously, with no jetting panel. FIG. 4B has a jetting panel located 10 mm below (upstream from) the wick. FIG. 4C has the same jetting panel 5 mm below the wick. FIG. 4D has the same jetting panel 2.5 mm below the wick. As can be seen from FIGS. 4B-4D, the jetting panel has an arrangement of apertures shaped and directed in order to promote jetting from the downstream face of the panel and therefore to promote turbulent flow. Accordingly, the jetting panel can introduce turbulence downstream, and the panel causes higher level of turbulence near the wick when it is positioned closer to the wick. As shown in FIGS. 4A-4D, the four geometries gave turbulence intensities of 0.55%, 0.77%, 1.06% and 1.34%, respectively, with FIG. 4A being the least turbulent, and FIG. 4D being the most turbulent.

For each of FIGS. 4A-4D, there are shown three modelling images. The image on the left shows the original image (color in the original), the central image shows a greyscale version of the image and the right hand image shows a black and white version of the image. As will be appreciated, each version of the image highlights slightly different features of the flow. Together, they give a reasonable picture of the flow conditions at the wick.

These four devices were operated to generate aerosols following the procedure explained above (the first example) using a flow rate of 1.3 lpm and the generated aerosols were tested for particle size in the Spraytec laser diffraction system.

### Third Example: High Temperature Testing

This experiment of a third example aimed to investigate the influence of inflow air temperature on aerosol particle size, in order to investigate the effect of vapor cooling rate on aerosol generation.

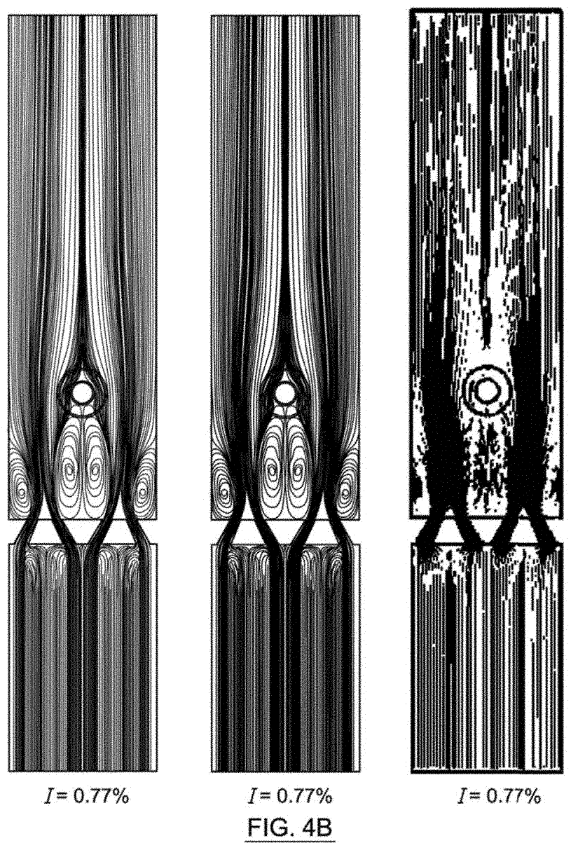

The experimental set up of the third example is shown in FIG. 5. The testing used a Carbolite Gero EHA 12300B tube furnace 3210 with a quartz tube 3220 to heat up the air. Hot air in the tube furnace was then led into a transparent housing 3158 that contains the EVP device 3150 to be tested. A thermocouple meter 3410 was used to assess the temperature of the air pulled into the EVP device. Once the EVP device was activated, the aerosol was pulled into the Spraytec laser diffraction system 3310 via a silicone connector 3320 for particle size measurement.

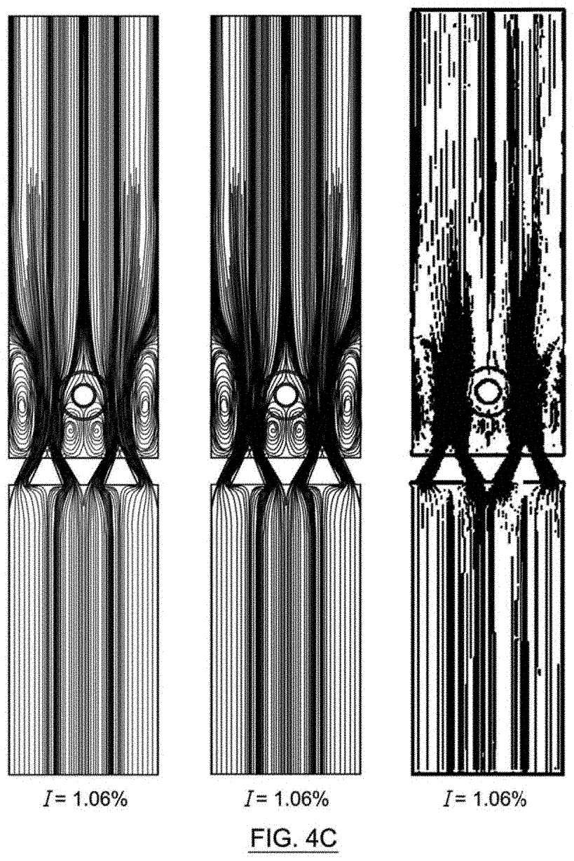

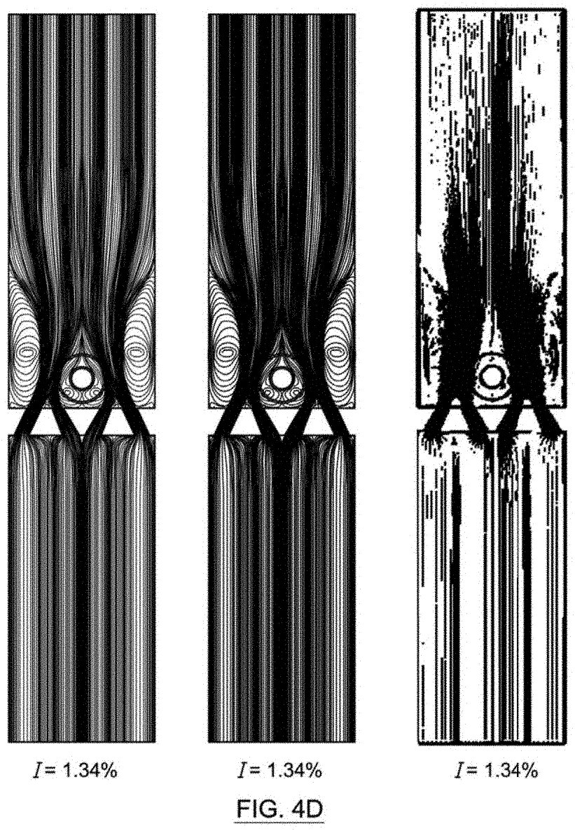

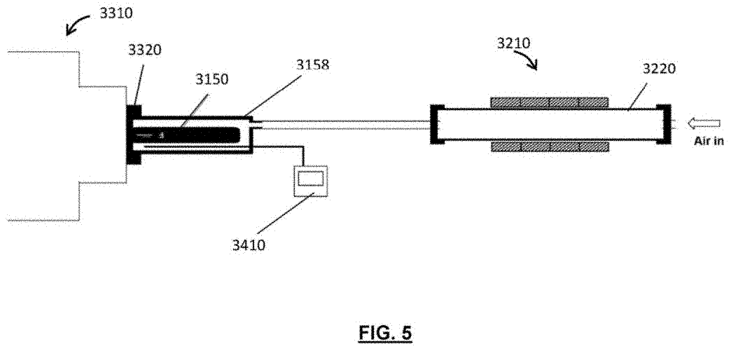

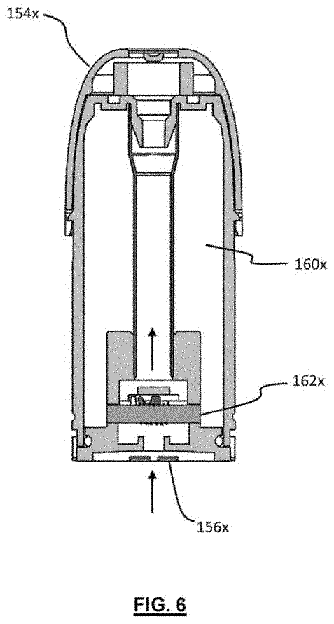

Three smoking substitute apparatuses (referred to as “pods”) were tested in the study: pod 1 is the commercially available “myblu optimised” pod (FIG. 6); pod 2 is a pod featuring an extended inflow path upstream of the wick (FIG. 7); and pod 3 is pod with the wick located in a stagnant vaporization chamber and the inlet air bypassing the vaporization chamber but entraining the vapor from an outlet of the vaporization chamber (FIGS. 8A and 8B).

Pod 1, shown in longitudinal cross sectional view (in the width plane) in FIG. 6, has a main housing that defines a tank 160x holding an e-liquid aerosol precursor. Mouthpiece 154x is formed at the upper part of the pod. Electrical contacts 156x are formed at the lower end of the pod. Wick 162x is held in a vaporization chamber. The air flow direction is shown using arrows.

Pod 2, shown in longitudinal cross sectional view (in the width plane) in FIG. 7, has a main housing that defines a tank 160y holding an e-liquid aerosol precursor. Mouthpiece 154y is formed at the upper part of the pod. Electrical contacts 156y are formed at the lower end of the pod. Wick 162y is held in a vaporization chamber. The air flow direction is shown using arrows. Pod 2 has an extended inflow path (plenum chamber 157y) with a flow conditioning element 159y, configured to promote reduced turbulence at the wick 162y.

FIG. 8A shows a schematic longitudinal cross sectional view of pod 3. FIG. 8B shows a schematic longitudinal cross sectional view of the same pod 3 in a direction orthogonal to the view taken in FIG. 8A. Pod 3 has a main housing that defines a tank 160z holding an e-liquid aerosol precursor. Mouthpiece 154z is formed at the upper part of the pod. Electrical contacts 156z are formed at the lower end of the pod. Wick 162z is held in a vaporization chamber. The air flow direction is shown using arrows. Pod 3 uses a stagnant vaporizer chamber, with the air inlets bypassing the wick and picking up the vapor/aerosol downstream of the wick.

All three pods were filled with the same e-liquid (1.6% freebase nicotine, 65:35 PG/VG ratio, no added flavor). Three experiments of the third example were carried out for each pod: 1) standard measurement in ambient temperature; 2) only the inlet air was heated to 50° C.; and 3) both the inlet air and the pods were heated to 50° C. Five repetition runs were carried out for each experiment and the Dv50 results were taken and averaged.

Modelling Work

In the following examples, modelling work was performed using COMSOL Multiphysics 5.4, engaged physics include: 1) laminar single-phase flow; 2) turbulent single-phase flow; 3) laminar two-phase flow; 4) heat transfer in fluids; and (5) particle tracing. Data analysis and data visualization were mostly completed in MATLAB R2019a.

### Fourth Example: Velocity Modelling

Air velocity in the vicinity of the wick is believed to play an important role in affecting particle size. In the first example, the air velocity was calculated by dividing the flow rate by the intersection area, which is referred to as “calculated velocity” in a fourth example. This involves a very crude simplification that assumes velocity distribution to be homogeneous across the intersection area.

In order to increase reliability of the fourth example, computational fluid dynamics (CFD) modelling was performed to obtain more accurate velocity values:

The average velocity in the vicinity of the wick (defined as a volume from the wick surface to 1 mm away from the wick surface)

The maximum velocity in the vicinity of the wick (defined as a volume from the wick surface to 1 mm away from the wick surface)

TABLE 2
Average and maximum velocity in the vicinity of wick surface obtained
from CFD modelling.

| | | | Calculated | Average | Maximum |
|---|---|---|---|---|---|
| | Tube size | Flow rate | velocity* | velocity** | Velocity** |
| | [mm] | [Ipm] | [m/s] | [m/s] | [m/s] |
| 1.3 Ipm | 4.5 | 1.3 | 1.17 | 0.99 | 1.80 |
| constant | 6 | 1.3 | 0.71 | 0.66 | 1.22 |
| flow rate | 7 | 1.3 | 0.56 | 0.54 | 1.01 |
| | 8 | 1.3 | 0.47 | 0.46 | 0.86 |
| | 10 | 1.3 | 0.35 | 0.35 | 0.66 |
| | 12 | 1.3 | 0.28 | 0.27 | 0.54 |
| | 20 | 1.3 | 0.15 | 0.15 | 0.32 |
| | 50 | 1.3 | 0.06 | 0.05 | 0.12 |
| 2.0 Ipm | 4.5 | 2.0 | 1.81 | 1.52 | 2.73 |
| constant | 5 | 2.0 | 1.48 | 1.31 | 2.39 |
| flow rate | 6 | 2.0 | 1.09 | 1.02 | 1.87 |
| | 8 | 2.0 | 0.72 | 0.71 | 1.31 |
| | 12 | 2.0 | 0.42 | 0.44 | 0.83 |
| | 20 | 2.0 | 0.23 | 0.24 | 0.49 |
| | 50 | 2.0 | 0.09 | 0.08 | 0.19 |

*Calculated by dividing flow rate with intersection area
**Obtained from CFD modelling

The CFD model uses a laminar single-phase flow setup. For each experiment, the outlet was configured to a corresponding flowrate, the inlet was configured to be pressure-controlled, the wall conditions were set as “no slip”. A 1 mm wide ring-shaped domain (wick vicinity) was created around the wick surface, and domain probes were implemented to assess the average and maximum magnitudes of velocity in this ring-shaped wick vicinity domain.

The CFD model of the fourth example outputs the average velocity and maximum velocity in the vicinity of the wick for each set of experiments carried out in the first example. The outcomes are reported in Table 2.

### Fifth Example: Turbulence Modelling

Turbulence intensity (I) is a quantitative value that represents the level of turbulence in a fluid flow system. It is defined as the ratio between the root-mean-square of velocity fluctuations, u′, and the Reynolds-averaged mean flow velocity, U:

$$1 = \frac{{u}^{′}}{U} = \frac{\sqrt{\frac{1}{3} ( {u}_{x}^{′2} + {u}_{y}^{′2} + {u}_{z}^{′2} )}}{\sqrt{{\overline{{u}_{x}}}^{2} + {\overline{{u}_{y}}}^{2} + {\overline{{u}_{z}}}^{2}}} = \frac{\sqrt{\frac{1}{3} [ {( {u}_{x} - \overline{{u}_{x}} )}^{2} + {( {u}_{y} - \overline{{u}_{y}} )}^{2} + + {( {u}_{z} - \overline{{u}_{z}} )}^{2} ]}}{\sqrt{{\overline{{u}_{x}}}^{2} + {\overline{{u}_{y}}}^{2} + {\overline{{u}_{z}}}^{2}}} ,$$

where uₓ, u_{y} and u_{z} are the x-, y- and z-components of the velocity vector, u̅ₓ̅, u̅_̅{̅y̅}̅, and u̅_̅{̅z̅}̅, represent the average velocities along three directions.

Higher turbulence intensity values represent higher levels of turbulence. As a rule of thumb, turbulence intensity below 1% represents a low-turbulence case, turbulence intensity between 1% and 5% represents a medium-turbulence case, and turbulence intensity above 5% represents a high-turbulence case.

In a fifth example, turbulence intensity was obtained from CFD simulation using turbulent single-phase setup in COMSOL Multiphysics. For each of the four experiments explained in the second example, above, the outlet was set to 1.3 lpm, the inlet was set to be pressure-controlled, and all wall conditions were set to be “no slip”.

Turbulence intensity of the fifth example was assessed within the volume up to 1 mm away from the wick surface (defined as the wick vicinity domain). For the four experiments explained in the second example, the turbulence intensities are 0.55%, 0.77%, 1.06% and 1.34%, respectively, as also shown in FIGS. 4A-4D.

### Sixth Example: Cooling Rate Modelling

The cooling rate modelling of the sixth example involves three coupling models in COMSOL Multiphysics: 1) laminar two-phase flow; 2) heat transfer in fluids, and 3) particle tracing. The model is setup in three steps:

(1) Set Up Two Phase Flow Model

Laminar mixture flow physics was selected for the sixth example. The outlet was configured in the same way as in the fourth example. However, this model of the sixth example includes two fluid phases released from two separate inlets: the first one is the vapor released from wick surface, at an initial velocity of 2.84 cm/s (calculated based on 5 mg total particulate mass over 3 seconds puff duration) with initial velocity direction normal to the wick surface; the second inlet is air influx from the base of tube, the rate of which is pressure-controlled.

(2) Set Up Two-Way Coupling with Heat Transfer Physics

The inflow and outflow settings in heat transfer physics was configured in the same way as in the two-phase flow model. The air inflow was set to 25° C., and the vapor inflow was set to 209° C. (boiling temperature of the e-liquid formulation). In the end, the heat transfer physics is configured to be two-way coupled with the laminar mixture flow physics. The above model reaches steady state after approximately 0.2 second with a step size of 0.001 second.

(3) Set Up Particle Tracing

A wave of 2000 particles were release from wick surface at t=0.3 second after the two-phase flow and heat transfer model has stabilized. The particle tracing physics has one-way coupling with the previous model, which means the fluid flow exerts dragging force on the particles, whereas the particles do not exert counterforce on the fluid flow. Therefore, the particles function as moving probes to output vapor temperature at each timestep.

The model of the sixth example outputs average vapor temperature at each time steps. A MATLAB script was then created to find the time step when the vapor cools to a target temperature (50° C. or 75° C.), based on which the vapor cooling rates were obtained (Table 3).

TABLE 3
Average vapor cooling rate obtained from Multiphysics modelling.

| | | | Cooling rate to | Cooling rate to |
|---|---|---|---|---|
| | Tube size | Flow rate | 50° C. | 75° C. |
| | [mm] | [Ipm] | [° C./ms] | [° C./ms] |
| 1.3 Ipm | 4.5 | 1.3 | 11.4 | 44.7 |
| constant | 6 | 1.3 | 5.48 | 14.9 |
| flow rate | 7 | 1.3 | 3.46 | 7.88 |
| | 8 | 1.3 | 2.24 | 5.15 |
| | 10 | 1.3 | 1.31 | 2.85 |
| | 12 | 1.3 | 0.841 | 1.81 |
| | 20 | 1.3 | 0* | 0.536 |
| | 50 | 1.3 | 0 | 0 |
| 2.0 Ipm | 4.5 | 2.0 | 19.9 | 670 |
| constant | 5 | 2.0 | 13.3 | 67 |
| flow rate | 6 | 2.0 | 8.83 | 26.8 |
| | 8 | 2.0 | 3.61 | 8.93 |
| | 12 | 2.0 | 1.45 | 3.19 |
| | 20 | 2.0 | 0.395 | 0.761 |
| | 50 | 2.0 | 0 | 0 |

*Zero cooling rate when the average vapor temperature is still above target temperature after 0.5 second

Results and Discussions

Particle size measurement results for the rectangular tube testing example above (the first example) are shown in Table 4. For every tube size and flow rate combination, five repetition runs were carried out in the Spraytec laser diffraction system. The Dv50 values from five repetition runs were averaged, and the standard deviations were calculated to indicate errors, as shown in Table 4.

In this section, the roles of different factors affecting aerosol particle size will be discussed based on experimental and modelling results.

TABLE 4
Particle size measurement results for the rectangular tube testing.

| | | | | Dv50 |
|---|---|---|---|---|
| | Tube | Flow | Dv50 | standard |
| | size | rate | average | deviation |
| | [mm] | [Ipm] | [μm] | [μm] |
| 1.3 Ipm | 4.5 | 1.3 | 0.971 | 0.125 |
| constant flow | 6 | 1.3 | 1.697 | 0.341 |
| rate | 7 | 1.3 | 2.570 | 0.237 |
| | 8 | 1.3 | 2.705 | 0.207 |
| | 10 | 1.3 | 2.783 | 0.184 |
| | 12 | 1.3 | 3.051 | 0.325 |
| | 20 | 1.3 | 3.116 | 0.354 |
| | 50 | 1.3 | 3.161 | 0.157 |
| 2.0 Ipm | 4.5 | 2.0 | 0.568 | 0.039 |
| constant flow | 5 | 2.0 | 0.967 | 0.315 |
| rate | 6 | 2.0 | 1.541 | 0.272 |
| | 8 | 2.0 | 1.646 | 0.363 |
| | 12 | 2.0 | 3.062 | 0.153 |
| | 20 | 2.0 | 3.566 | 0.260 |
| | 50 | 2.0 | 3.082 | 0.440 |
| 1.0 m/s | 5.0 | 1.4 | 1.302 | 0.187 |
| constant air | 8 | 2.8 | 1.303 | 0.468 |
| velocity | 20 | 8.6 | 1.463 | 0.413 |

### Seventh Example: Decouple the Factors Affecting Particle Size

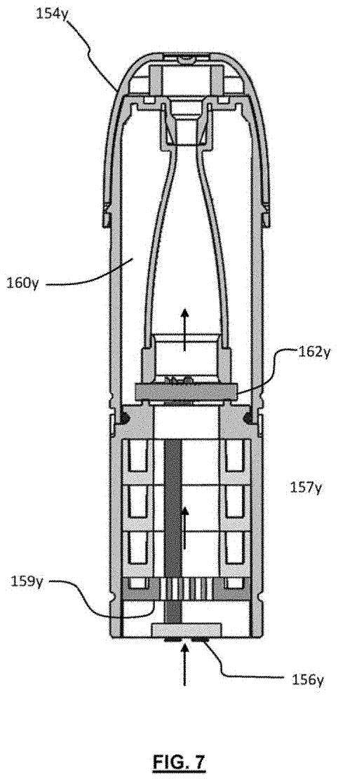

The particle size (Dv50) experimental results of a seventh example are plotted against calculated air velocity in FIG. 9. The graph shows a strong correlation between particle size and air velocity.

Different size tubes were tested at two flow rates: 1.3 lpm and 2.0 lpm. Both groups of data show the same trend that slower air velocity leads to larger particle size. The conclusion was made more convincing by the fact that these two groups of data overlap well in FIG. 9: for example, the 6 mm tube delivered an average Dv50 of 1.697 μm when tested at 1.3 lpm flow rate, and the 8 mm tube delivered a highly similar average Dv50 of 1.646 μm when tested at 2.0 lpm flow rate, as they have similar air velocity of 0.71 and 0.72 m/s, respectively.

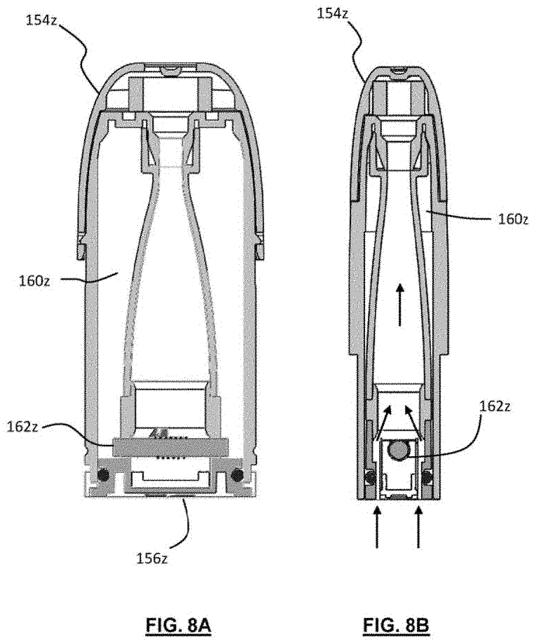

In addition, FIG. 10 shows the results of three experiments of the seventh example, with highly different setup arrangements: 1) 5 mm tube measured at 1.4 lpm flow rate with Reynolds number of 155; 2) 8 mm tube measured at 2.8 lpm flow rate with Reynolds number of 279; and 3) 20 mm tube measured at 8.6 lpm flow rate with Reynolds number of 566. It is relevant that these setup arrangements have one similarity: the air velocities are all calculated to be 1 m/s. FIG. 10 shows that, although these three sets of experiments have different tube sizes, flow rates and Reynolds numbers, they all delivered similar particle sizes, as the air velocity was kept constant. These three data points were also plotted out in FIG. 9 (1 m/s data with star marks) and they tie in nicely into particle size-air velocity trendline.

The above results of the seventh example lead to a strong conclusion that air velocity is an important factor affecting the particle size of EVP devices. Relatively large particles are generated when the air travels with slower velocity around the wick. It can also be concluded that flow rate, tube size and Reynolds number are not necessarily independently relevant to particle size, providing the air velocity is controlled in the vicinity of the wick.

### Eighth Example: Further Consideration of Velocity

In FIG. 9 the “calculated velocity” was obtained by dividing the flow rate by the intersection area, which is a crude simplification that assumes a uniform velocity field. In order to increase reliability of the work, CFD modelling has been performed to assess the average and maximum velocities in the vicinity of the wick. In an eighth example, the “vicinity” was defined as a volume from the wick surface up to 1 mm away from the wick surface.

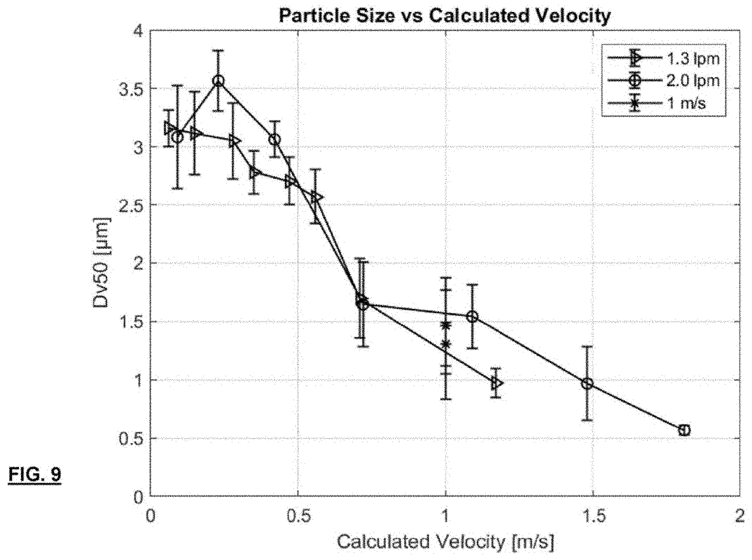

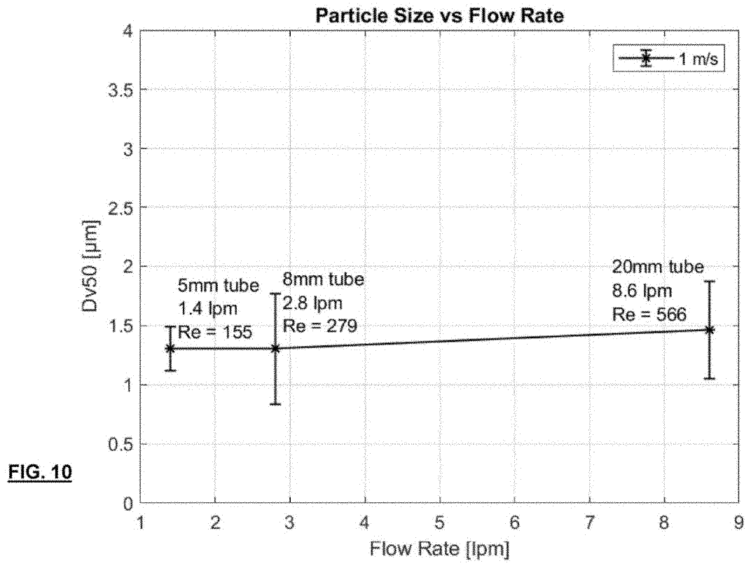

The particle size measurement data of the eighth example were plotted against the average velocity (FIG. 11) and maximum velocity (FIG. 12) in the vicinity of the wick, as obtained from CFD modelling.

The data in these two graphs indicates that in order to obtain an aerosol with Dv50 larger than 1 μm, the average velocity should be less than or equal to 1.2 m/s in the vicinity of the wick and the maximum velocity should be less than or equal to 2.0 m/s in the vicinity of the wick.

Furthermore, in order to obtain an aerosol with Dv50 of 2 μm or larger, the average velocity should be less than or equal to 0.6 m/s in the vicinity of the wick and the maximum velocity should be less than or equal to 1.2 m/s in the vicinity of the wick.

It is considered that typical commercial EVP devices deliver aerosols with Dv50 around 0.5 μm, and there is no commercially available device that can deliver aerosol with Dv50 exceeding 1 μm. It is considered that typical commercial EVP devices have average velocity of 1.5-2.0 m/s in the vicinity of the wick.

### Ninth Example: The Role of Turbulence

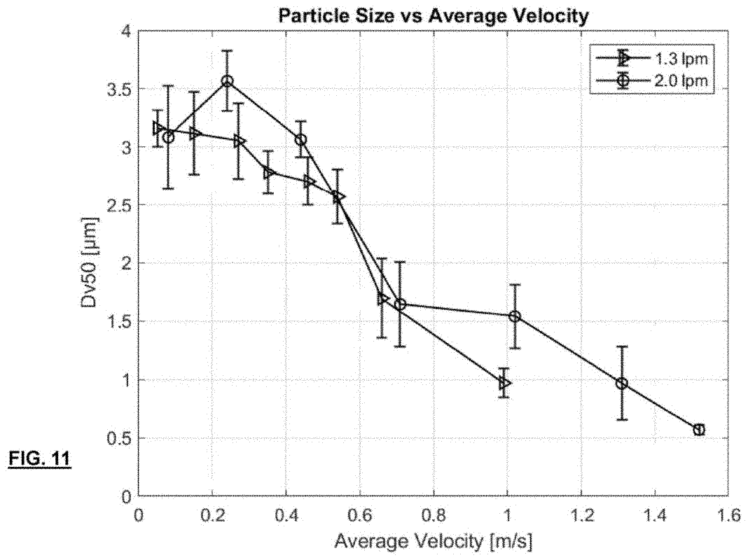

The role of turbulence has been investigated in terms of turbulence intensity in a ninth example, which is a quantitative characteristic that indicates the level of turbulence. In the ninth example, four tubes of different turbulence intensities were used to general aerosols which were measured in the Spraytec laser diffraction system. The particle size (Dv50) experimental results of the ninth example are plotted against turbulence intensity in FIG. 13.

The graph suggests a correlation between particle size and turbulence intensity, that lower turbulence intensity is beneficial for obtaining larger particle size. It is noted that when turbulence intensity is above 1% (medium-turbulence case), there are relatively large measurement fluctuations. In FIG. 13, the tube with a jetting panel 10 mm below the wick has the largest error bar, because air jets become unpredictable near the wick after traveling through a long distance.

The results of the ninth example clearly indicate that laminar air flow is favorable for the generation of aerosols with larger particles, and that the generation of large particle sizes is jeopardized by introducing turbulence. In FIG. 13, the 12 mm standard rectangular tube (without jetting panel) delivers above 3 μm particle size (Dv50). The particle size values reduced by at least a half when jetting panels were added to introduce turbulence.

### Tenth Example: Vapor Cooling Rate

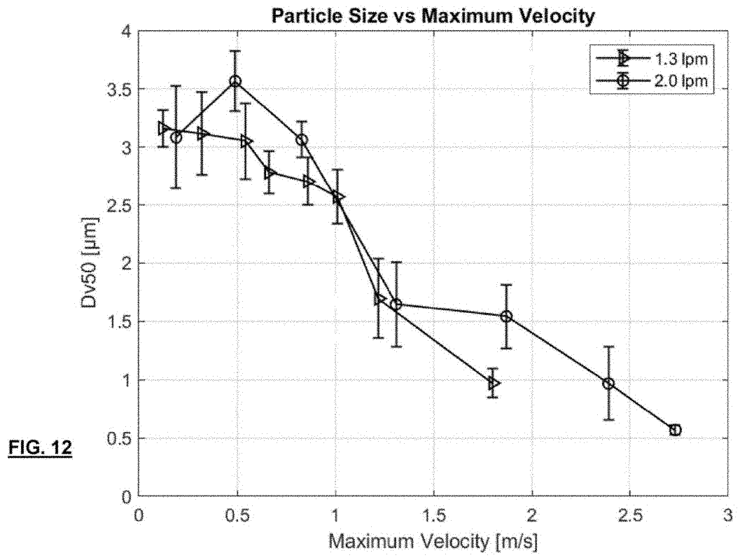

FIG. 14 shows the high temperature testing results of a tenth example. Larger particle sizes were observed from all 3 pods when the temperature of inlet air increased from room temperature (23° C.) to 50° C. When the pods were heated as well, two of the three pods saw even larger particle size measurement results, while pod 2 was unable to be measured due to significant amount of leakage.

Without wishing to be bound by theory, the results of the tenth example are in line with the inventors' insight that control over the vapor cooling rate provides an important degree of control over the particle size of the aerosol. As reported above, the use of a slow air velocity can have the result of the formation of an aerosol with large Dv50. It is considered that this is due to slower air velocity allowing a slower cooling rate of the vapor.

Another conclusion related to laminar flow can also be explained by a cooling rate theory of the tenth example: laminar flow allows slow and gradual mixing between cold air and hot vapor, which means the vapor can cool down in slower rate when the airflow is laminar, resulting in larger particle size.

The results in FIG. 14 further validate this cooling rate theory of the tenth example: when the inlet air has higher temperature, the temperature difference between hot vapor and cold air becomes smaller, which allows the vapor to cool down at a slower rate, resulting in larger particle size; when the pods were heated as well, this mechanism was exaggerated even more, leading to an even slower cooling rate and an even larger particle size.

### Eleventh Example: Further Consideration of Vapor Cooling Rate

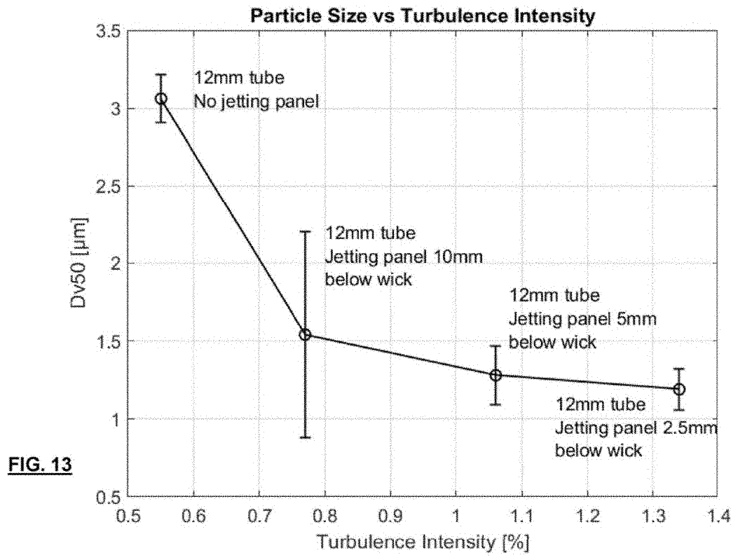

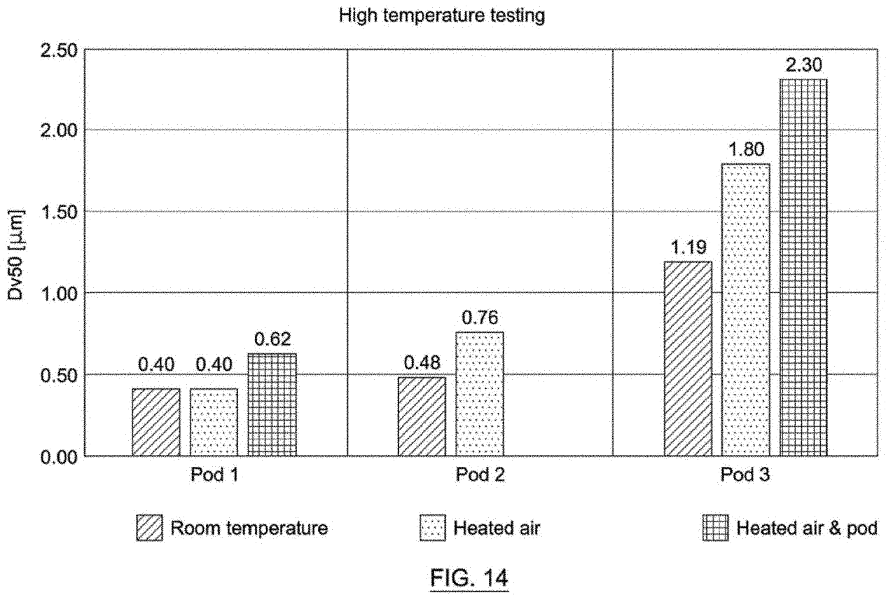

In the sixth example, the vapor cooling rates for each tube size and flow rate combination were obtained via multiphysics simulation. In FIG. 15 and FIG. 16, the particle size measurement results were plotted against vapor cooling rate to 50° C. and 75° C., respectively.

The data in these graphs indicates that in order to obtain an aerosol with Dv50 larger than 1 μm, the apparatus should be operable to require more than 16 ms for the vapor to cool to 50° C., or an equivalent (simplified to an assumed linear) cooling rate being slower than 10° C./ms. From an alternative viewpoint, in order to obtain an aerosol with Dv50 larger than 1 μm, the apparatus should be operable to require more than 4.5 ms for the vapor to cool to 75° C., or an equivalent (simplified to an assumed linear) cooling rate slower than 30° C./ms.

Furthermore, in order to obtain an aerosol with Dv50 of 2 μm or larger, the apparatus should be operable to require more than 32 ms for the vapor to cool to 50° C., or an equivalent (simplified to an assumed linear) cooling rate being slower than 5° C./ms. From an alternative viewpoint, in order to obtain an aerosol with Dv50 of 2 μm or larger, the apparatus should be operable to require more than 13 ms for the vapor to cool to 75° C., or an equivalent (simplified to an assumed linear) cooling rate slower than 10° C./ms.

Conclusions of Particle Size Experimental Work

In the above example, particle size (Dv50) of aerosols generated in a set of rectangular tubes was studied in order to decouple different factors (flow rate, air velocity, Reynolds number, tube size) affecting aerosol particle size. It is considered that air velocity is an important factor affecting particle size—slower air velocity leads to larger particle size. When air velocity was kept constant, the other factors (flow rate, Reynolds number, tube size) has low influence on particle size.

The role of turbulence was also investigated in the above examples. It is considered that laminar air flow favors generation of large particles, and introducing turbulence deteriorates (reduces) the particle size.

Modelling methods were used in some of the above examples to simulate the average air velocity, the maximum air velocity, and the turbulence intensity in the vicinity of the wick. A COMSOL model with three coupled physics has also been developed to obtain the vapor cooling rate.

All experimental and modelling results of the above examples support a cooling rate theory that slower vapor cooling rate is a significant factor in ensuring larger particle size. Slower air velocity, laminar air flow and higher inlet air temperature lead to larger particle size, because they all allow vapor to cool down at slower rates.

The features disclosed in the foregoing description, or in the following claims, or in the accompanying drawings, or in the above examples, expressed in their specific forms or in terms of a means for performing the disclosed function, or a method or process for obtaining the disclosed results, as appropriate, may, separately, or in any combination of such features, be utilized for realizing the disclosure in diverse forms thereof.

While the disclosure has been described in conjunction with the exemplary embodiments and examples described above, many equivalent modifications and variations will be apparent to those skilled in the art when given this disclosure. Accordingly, the exemplary embodiments and examples of the disclosure set forth above are considered to be illustrative and not limiting. Various changes to the described embodiments may be made without departing from the spirit and scope of the disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the disclosure that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

### ILLUSTRATIVE EMBODIMENTS

In the following numbered “clauses” are set out statements of broad combinations of novel and inventive features of the present disclosure herein disclosed.

Development A

A1. A smoking substitute apparatus (150a) comprising:
an air inlet (176);
a first passage (170) leading from the air inlet (176) to a first outlet (174);
an aerosol generator arranged in fluid communication with the first passage (170), the aerosol generator being operable to generate an aerosol from an aerosol precursor (160), to flow in use along the first passage (170) downstream of the aerosol generator for inhalation by a user drawing on the first outlet (174),
characterized in that
the apparatus (150a) further comprises a second passage (180) leading from the air inlet (176) to a second outlet (184) separate from the first outlet (174), wherein the second passage (180) bypasses the first passage (170) downstream of the aerosol generator.

A2. A smoking substitute apparatus (150a) according to clause A1, wherein:
the first passage (170) comprises a vaporization chamber in which the aerosol generator is arranged, the vaporization chamber being bypassed by the second passage (180), and wherein
the vaporization chamber has a larger cross sectional diameter than a downstream part of the first passage (170).

A3. A smoking substitute apparatus (150a) according to either of clauses A1 and A2, wherein: the aerosol generator comprises a heater operable to generate the aerosol from the aerosol precursor (160).

A4. A smoking substitute apparatus (150a) according to any preceding clause A1 to A3, wherein: the aerosol generator comprises a porous wick which, in use, wicks aerosol precursor (160) from a reservoir (152) to the first passage (170) for entrainment in air flowing downstream of the aerosol generator.

A5. A smoking substitute apparatus (150a) according to clause A4 as dependent on clause A3, wherein: the heater comprises a heating filament (164) that is wound around a portion of the porous wick (162).

A6. A smoking substitute apparatus (150a) according to any preceding clause A1 to A5, wherein: the part of the first passage (170) bypassed by the second passage (180) comprises a flow conditioning apparatus (200) arranged upstream of the aerosol generator which, when the smoking substitute apparatus (150a) is in use, reduces turbulence in flow at the aerosol generator.

A7. A smoking substitute apparatus (150a) according to clause A6, wherein: the flow conditioning apparatus (200) comprises a mesh arranged in the first passage (170) such that, in use, the flow generated by a user drawing on the first outlet (174) passes through the mesh.

A8. A smoking substitute apparatus (150a) according to any preceding clause A1 to A7, wherein: the first passage (170) and the second passage (180) are configured such that, in use, the flow rate in the first passage (170) is more than 1/20 of the flow rate in the second passage (180).

A9. A smoking substitute apparatus (150a) according to clause A8, wherein: the first passage (170) and the second passage (180) are configured such that, in use, the flow rate in the first passage (170) is less than twice of the flow rate in the second passage (180).

A10. A smoking substitute apparatus (150a) according to any preceding clause A1 to A9, wherein: in use, the d₅₀ particle size of the aerosol particles generated by the aerosol generator is greater than 1 μm.

A11. A smoking substitute apparatus (150a) according to any preceding clause A1 to A10, wherein: in use, the d₅₀ particle size of the aerosol particles generated by the aerosol generator is less than 10 μm.

A12. A smoking substitute apparatus (150a) according to any preceding clause A1 to A11, wherein: in use, the span of particle size distribution, defined as (d90−d10)/d50, is less than 20.

A13. A smoking substitute apparatus (150a) according to any one of clause A1 to clause A12, wherein the smoking substitute apparatus (150a) is comprised by or within a cartridge configured for engagement with a base unit (120), the cartridge and base unit together forming a smoking substitute system (110).

A14. A smoking substitute system (110) comprising: a base unit (120), and a smoking substitute apparatus (150a) according to clause A13, wherein the smoking substitute apparatus (150a) is removably engageable with the base unit (120).

A15. A method of using a smoking substitute apparatus according to any one of clauses A1 to A12 to generate an aerosol.

Development B

B1. A smoking substitute apparatus comprising:
an air inlet and an outlet;
an aerosol generator; and
an enclosure defining a vaporization chamber, wherein the enclosure at least partially encloses the aerosol generator;
wherein the aerosol generator comprises a heater, the aerosol generator being operable to generate an aerosol by vaporizing an aerosol precursor, the aerosol being for entrainment in an air flow flowing in use from the air inlet to the outlet when a user draws air through the apparatus,
wherein, in use, at least a part of the air flow from the air inlet to the outlet bypasses the vaporization chamber, and
wherein at least part of the enclosure adjacent the heater is formed from plastics material, there being provided a heat shield between said part of the enclosure and the heater in the vaporization chamber.

B2. A smoking substitute apparatus according to clause B1, further comprising a housing disposed externally of the enclosure, the housing being formed at least in part from a plastics material.

B3. A smoking substitute apparatus according to clause B1 or clause B2, wherein the closest distance between said part of the enclosure and the heater, is at most 2 mm.

B4. A smoking substitute apparatus according to any one of clauses B1 to B3 wherein the heat shield is formed of a material having a thermal degradation temperature at least 100° C. higher than that of the plastics material forming the enclosure.

B5. A smoking substitute apparatus according to any one of clauses B1 to B4 wherein the heat shield is formed of a material selected from metals, thermosetting polymers and ceramics and composites thereof.

B6. A smoking substitute apparatus according to any one of clauses B1 to B5 wherein the heat shield presents to the heater a heat-absorbing surface having an area of at least twice as large as a plan view projection of the heater onto the heat shield.

B7. A smoking substitute apparatus according to clause B6 wherein the area of the heat-absorbing surface of the heat shield is at least 20 mm².

B8. A smoking substitute apparatus according to any one of clauses B1 to B7 wherein the vaporization chamber has an elongate shape, wider in a first direction orthogonal to a longitudinal axis of the apparatus compared with a second direction orthogonal to the first direction and to the longitudinal axis of the apparatus, the heater extending along the first direction and the heat shield extending between the heater and the enclosure substantially parallel to the first direction.

B9. A smoking substitute apparatus according to any one of clauses B1 to B8 wherein there are provided first and second heat shield plates disposed on opposing sides of the heater.

B10. A smoking substitute apparatus according to any one of clauses B1 to B9 wherein, in use, substantially all of the air flow from the air inlet to the outlet bypasses the vaporization chamber, the vaporization chamber having a vaporization chamber outlet in communication with a passage along which air flows from the air inlet to the outlet.

B11. A smoking substitute apparatus according to clause B10 wherein the vaporization chamber is substantially sealed against air flow except for the vaporization chamber outlet.

B12. A smoking substitute apparatus according to any one of clauses B1 to B9 wherein a first passage leads from the air inlet to the outlet, the aerosol generator being arranged in fluid communication with the first passage, the apparatus further comprises a second passage leading from the air inlet to the outlet, wherein the second passage bypasses the first passage downstream of the aerosol generator.

B13. A smoking substitute apparatus (150a) according to any one of clauses B1 to B12, wherein the smoking substitute apparatus (150a) is comprised by or within a cartridge configured for engagement with a base unit (120), the cartridge and base unit together forming a smoking substitute system (110).

B14. A smoking substitute system (110) comprising: a base unit (120), and a smoking substitute apparatus (150a) according to clause B13, wherein the smoking substitute apparatus (150a) is removably engageable with the base unit (120).

B15. A method of using a smoking substitute apparatus according to any one of clauses B1 to B13 to generate an aerosol.

Development C

C1. A smoking substitute apparatus comprising:
a housing having a first end and a second end, and at least one sidewall:
an air inlet provided at said sidewall of the housing;
an outlet provided at the second end of the housing;
a main flow channel and a bypass flow channel extending in different directions from the air inlet, the main flow channel and the bypass flow channel being in communication with the outlet;
an aerosol generator positioned in the main flow channel for generating aerosol from an aerosol precursor;
wherein in use, air flowing through the air inlet is split into a main air flow and a bypass air flow, the main and bypass air flows flowing along the main flow channel and the bypass flow channel respectively, wherein immediately downstream of the air inlet, the main air flow travels towards one of the first and second ends of the housing, and the bypass air flow travels towards the other of the first and second ends of the housing.

C2. A smoking substitute apparatus according to clause C1, wherein in use, immediately downstream of the air inlet, the main air flow travels towards the first end of the housing, and the bypass air flow travels towards the second end of the housing.

C3. The smoking substitute apparatus according to any one of the previous clauses C1 to C2, wherein the aerosol generator includes a heater.

C4. The smoking substitute apparatus according to any one of the previous clauses C1 to C3, wherein the aerosol precursor is a liquid.

C5. The smoking substitute apparatus according to any one of the previous clauses C1 to C4, wherein the air inlet is disposed substantially at a midpoint of the sidewall.

C6. The smoking substitute apparatus according to any one of the previous clauses C1 to C5, wherein the air inlet is defined by a T-junction between an opening in the sidewall of the housing and the main and bypass flow channels.

C7. The smoking substitute apparatus according to any one of the previous clauses C1 to C6, wherein, downstream of the aerosol generator, the main air flow is in the opposite direction to the main air flow upstream of the aerosol generator and proximate the air inlet.

C8. The smoking substitute apparatus according to any one of the previous clauses C1 to C7, wherein the smoking substitute apparatus is configured to generate an aerosol having a median droplet size, d50, of at least 1 μm.

C9. A smoking substitute device configured to engage with the smoking substitute apparatus according to any one of the previous clauses C1 to C8, wherein the device comprises a controller and a power source configured to energize the aerosol generator.

C10. A smoking substitute system for generating an aerosol, comprising: the smoking substitute apparatus according to any one of clauses C1 to C8; and the smoking substitute device according to clause C9.

C11. A method of generating an aerosol using the smoking substitute apparatus of any one of clauses C1 to C8, wherein the droplets of the aerosol have a median diameter, d50, of at least 1 μm.

Development D

D1. A smoking substitute apparatus (150a, 150b, 150c, 150d) comprising:
one or more air inlets (172, 176, 182);
one or more outlets (174, 184)
a first passage (170) leading from at least one of the air inlets (172, 176) to a first outlet (174) among the outlets;
an aerosol generator arranged in a vaporization chamber in the first passage (170), the aerosol generator comprising a heater (164) and being operable to generate an aerosol from an aerosol precursor (160), to flow in use along the first passage (170) downstream of the aerosol generator for inhalation by a user drawing on the first outlet (174),
a second passage (180) leading from at least one of the air inlets (176, 182) to at least one of the outlets (174, 184) wherein the second passage (180) bypasses the vaporization chamber of the first passage (170), and wherein the second passage comprises an auxiliary heater for heating air in the second passage (180).

D2. A smoking substitute apparatus (150a, 150b, 150c, 150d) according to clause D1, wherein: the auxiliary heater (190) for heating the air in the second passage (180) comprises an electrically heatable mesh.

D3. A smoking substitute apparatus (150a, 150b, 150c, 150d) according to any one of clauses D1 or D2, wherein: the auxiliary heater (190) for heating the air in the second passage (180) comprises an electrically heatable heating coil.

D4. A smoking substitute apparatus (150a, 150b, 150c, 150d) according to any preceding clause D1 to D3, wherein: the auxiliary heater (190) for heating the air in the second passage (180) is heatable independently from the heater (164) in the first passage (170).

D5. A smoking substitute apparatus (150a, 150b, 150c, 150d) according to any preceding clause D1 to D4, wherein: the auxiliary heater (190) for heating the air in the second passage (180) is heatable in combination with the heater (164) in the first passage (170).

D6. A smoking substitute apparatus (150b, 150d) according to any preceding clause D1 to D5, wherein: the smoking substitute apparatus (150b, 150d) comprises an air inlet (182) to the second passage (180) which is not spatially coterminous with an air inlet (172) to the first passage (170).

D7. A smoking substitute apparatus (150a, 150b) according to any preceding clause D1 to D6, wherein: the smoking substitute apparatus (150a, 150b) comprises an air outlet (184) from the second passage (180) which is not spatially coterminous with an air outlet (174) from the first passage (170).

D8. A smoking substitute apparatus (150a, 150b, 150c, 150d) according to any preceding clause D1 to D7, wherein: the part of the first passage (170) bypassed by the second passage (180) comprises a flow conditioning apparatus (200) arranged upstream of the aerosol generator which, when the smoking substitute apparatus (150a) is in use, reduces turbulence in flow at the aerosol generator.

D9. A smoking substitute apparatus (150a, 150b, 150c, 150d) according to any preceding clause D1 to D8, wherein: the first passage (170) and the second passage (180) are configured such that, in use, the flow rate in the first passage (170) is more than 1/20 of the flow rate in the second passage (180).

D10. A smoking substitute apparatus (150a, 150b, 150c, 150d) according to clause D9, wherein: the first passage (170) and the second passage (180) are configured such that, in use, the flow rate in the first passage (170) is less than twice of the flow rate in the second passage (180).

D11. A smoking substitute apparatus (150a, 150b, 150c, 150d) according to any preceding clause D1 to D10, wherein: in use, the d50 particle size of the aerosol particles generated by the aerosol generator is greater than 1 μm and less than 10 μm.

D12. A smoking substitute apparatus (150a, 150b, 150c, 150d) according to any preceding clause D1 to D11, wherein: in use, the span of particle size distribution, defined as (d90−d10)/d50, is less than 20.

D13. A smoking substitute apparatus (150a, 150b, 150c, 150d) according to any one of clause D1 to clause D12, wherein the smoking substitute apparatus (150a, 150b, 150c, 150d) is comprised by or within a cartridge configured for engagement with a base unit (120), the cartridge and base unit (120) together forming a smoking substitute system (110).

D14. A smoking substitute system (110) comprising: a base unit (120), and a smoking substitute apparatus (150a, 150b, 150c, 150d) according to clause D13, wherein the smoking substitute apparatus (150a, 150b, 150c, 150d) is removably engageable with the base unit.

D15. A method of using a smoking substitute apparatus according to any one of clauses D1 to D13 to generate an aerosol.

Development E

E1. A smoking substitute apparatus (150a, 150b, 150c) comprising:
an air inlet (176);
a vaporizer passage (170) leading from the air inlet (176) to an outlet (174);
an aerosol generator arranged in a vaporization chamber in the vaporizer passage (170), the aerosol generator being operable to generate an aerosol from an aerosol precursor (160), to flow in use along the vaporizer passage (170) downstream of the aerosol generator for inhalation by a user drawing on the apparatus, and
a bypass passage leading from the air inlet (176) to a bypass passage outlet at a junction (184a, 184b, 184c) arranged adjacent to the outlet (174), wherein the bypass passage (180) bypasses the vaporization chamber of the vaporizer passage (170), and wherein the bypass passage (180) meets the vaporizer passage (170) at the junction (184a, 184b, 184c) in a direction substantially perpendicular to a longitudinal axis of the vaporizer passage (170) at the junction (184a, 184b, 184c).

E2. A smoking substitute apparatus (150b, 150c) according to clause E1, comprising: a plurality of bypass passages (180), each extending from the air inlet (176) to the junction (184b, 184c), wherein a longitudinal axis of at least one of the bypass passage outlets at the junction (184b, 184c) is offset from the longitudinal axis of the vaporizer passage (170) at the junction.

E3. A smoking substitute device according to any preceding clause E1 to E2, comprising: a plurality of bypass passages (180), each extending from the air inlet (176) to the junction (184b, 184c), wherein a first bypass passage outlet at the junction (184b, 184c) is offset from a second bypass passage outlet at the junction (184b, 184c) along a direction substantially parallel to the longitudinal axis of the vaporizer passage (170) at the junction.

E4. A smoking substitute apparatus (150b, 150c) according to clause E2 or clause E3, wherein: the bypass passage outlets at the junction (184b, 184c) are arranged such that, in use, air drawn through the bypass passages (180) generates a vortex at or near the junction (184b, 184c) in air drawn through the vaporizer passage (170).

E5. A smoking substitute apparatus (150c) according to clause E3, wherein: the bypass passage outlets at the junction (184c) are arranged such that, in use, air drawn through the bypass passages (180) generates a plurality of counter-rotating vortices proximate to the junction (184c) in air drawn through the vaporizer passage (170).

E6. A smoking substitute apparatus (150a) according to any preceding clause E1 to E5, comprising: a plurality of bypass passages (180), each extending from the air inlet (176) to the junction (184a), wherein at least one pair of the bypass passage outlets are arranged at symmetrically opposed positions across the vaporizer passage (170).

E7. A smoking substitute apparatus (150a, 150b, 150c) according to any preceding clause E1 to E6, wherein: the aerosol generator comprises a heater operable to generate the aerosol from the aerosol precursor (160).

E8. A smoking substitute apparatus (150a, 150b, 150c) according to any preceding clause E1 to E7, wherein: the aerosol generator comprises a porous wick which, in use, wicks aerosol precursor (160) from a reservoir (152) to the vaporizer passage (170) for entrainment in air flowing downstream of the aerosol generator.

E9. A smoking substitute apparatus (150a, 150b, 150c) according to any preceding clause E1 to E8, wherein: the part of the vaporizer passage (170) bypassed by the bypass passage (180) comprises a flow conditioning apparatus (200) arranged upstream of the aerosol generator which, when the smoking substitute apparatus (150a) is in use, reduces turbulence in flow at the aerosol generator.

E10. A smoking substitute apparatus (150a, 150b, 150c) according to any preceding clause E1 to E9, wherein: the vaporizer passage (170) and the bypass passage (180) are configured such that, in use, the flow rate in the vaporizer passage (170) is more than 1/20 of the flow rate in the bypass passage (180) and less than twice of the flow rate in the bypass passage (180).

E11. A smoking substitute apparatus (150a, 150b, 150c) according to any preceding clause E1 to E10, wherein: in use, the d50 particle size of the aerosol particles generated by the aerosol generator is greater than 1 μm and less than 10 μm.

E12. A smoking substitute apparatus (150a) according to any preceding clause E1 to E11, wherein: in use, the span of particle size distribution, defined as (d90−d10)/d50, is less than 20.

E13. A smoking substitute apparatus (150a, 150b, 150c) according to any one of clauses E1 to E12, wherein the smoking substitute apparatus (150a, 150b, 150c) is comprised by or within a cartridge configured for engagement with a base unit (120), the cartridge and base unit together forming a smoking substitute system (110).

E14. A smoking substitute system (110) comprising:
a base unit (120), and
a smoking substitute apparatus (150a, 150b, 150c) according to clause E13, wherein the smoking substitute apparatus (150a, 150b, 150c) is removably engageable with the base unit.

E15. A method of using a smoking substitute apparatus according to any one of clauses E1 to E13 to generate an aerosol.

Development F

F1. A smoking substitute apparatus comprising:
a main flow passage formed between a main air inlet and a main outlet;
at least one bypass flow passage formed between a bypass air inlet and a bypass outlet, wherein air flows in use along the main flow passage and along the bypass flow passage for inhalation by a user drawing on the apparatus;
an aerosol generator operable to generate an aerosol from an aerosol precursor, the aerosol generator being in communication with the main flow passage;
wherein there is provided at least one flow regulator for varying a proportion of air flow through the bypass flow passage compared with air flow through the main flow passage, the flow regulator presenting a variable flow resistance to air flow in the bypass flow passage, said flow resistance depending on an air pressure difference across the flow regulator in view of a rate of flow demanded through the apparatus by the user.

F2. The smoking substitute apparatus of clause F1, wherein the main air inlet and the bypass air inlet are separate.

F3. The smoking substitute apparatus of clause F1, wherein the main outlet and the bypass outlet are constituted by a common outlet.

F4. The smoking substitute apparatus of any one of the previous clauses F1 to F3, wherein the flow regulator includes a resiliently deformable member configured to vary the flow resistance to air flow in the bypass passage by resiliently deforming to a variable extent depending on the air pressure difference across the flow regulator.

F5. The smoking substitute apparatus of clause F4, wherein the resiliently deformable member is an annular member attached circumferentially to an inner wall of the bypass flow passage.

F6. The smoking substitute apparatus of any one of the previous clauses F1 to F5, wherein the flow regulator includes a member hinged with respect to a wall of the bypass flow passage, the member being configured to vary the flow resistance to air flow in the bypass passage by hinging about the wall of the bypass flow passage so as to vary the cross sectional area of flow through the regulator.

F7. The smoking substitute apparatus of any one of the previous clauses F1 to F6, further including a mouthpiece having a bypass channel for engaging with the bypass passage so as to be in fluid communication therewith, and a main channel for engaging with the main passage so as to be in fluid communication therewith, wherein at least one flow regulator is situated in the bypass channel of the mouthpiece.

F8. The smoking substitute apparatus of any one of the previous clauses F1 to F7, wherein the aerosol generator includes a heater.

F9. The smoking substitute apparatus of any one of the previous clauses F1 to F8, wherein the aerosol precursor is a liquid.

F10. The smoking substitute apparatus of any one of the previous clauses F1 to F9, wherein the apparatus is configured to generate an aerosol having a median droplet size, d50, of at least 1 μm.

F11. A smoking substitute device configured to engage with the smoking substitute apparatus of any one of the previous clauses F1 to F10; wherein the device comprises a controller and a power source configured to energize the aerosol generator.

F12. A smoking substitute system for generating an aerosol, comprising: the smoking substitute apparatus according to any one of the previous clauses F1 to F10; and the smoking substitute device of clause F11.

F13. A method of generating an aerosol using the smoking substitute apparatus of any one of clauses F1 to F10, wherein droplets of the aerosol have a median diameter, d50, of at least 1 μm.

Development G

G1. A smoking substitute apparatus comprising:
a housing;
one or more outlets formed at the housing;
a main air inlet and a bypass air inlet respectively formed at the housing; wherein the one or more outlets are configured to be in fluid communication with the main air inlet and the bypass air inlet provided at the housing through a respective main flow channel and a bypass flow channel;
an aerosol generator positioned along the main flow channel for generating an aerosol from an aerosol precursor;
a bypass constriction region provided along the bypass flow channel, the bypass constriction presenting a smaller cross sectional area to flow than that of the bypass air inlet and that of the one or more outlets, the cross sectional area of the bypass constriction region determining the resistance to flow along the bypass flow channel and thereby the ratio of flow between the bypass flow in the bypass flow channel and the main flow in the main flow channel; and
optionally, the smoking substitute apparatus further comprising a main constriction region provided along the main flow channel, the main constriction presenting a smaller cross sectional area to flow than that of the main air inlet, wherein the cross sectional area of the main constriction region determines the resistance to flow along the main flow channel, and thereby the main and bypass constriction regions determine the ratio of flow between the bypass flow in the bypass flow channel and the main flow in the main flow channel, to provide the total rate of flow at the one or more outlets.

G2. The smoking substitute apparatus according to clause G1, wherein at least one of the main constriction region, if present, and the bypass constriction region includes a tapered region of the flow channel in which it is disposed, the cross sectional area of the channel tapering to a smaller cross sectional area than that of the respective air inlet of the flow channel.

G3. The smoking substitute apparatus according to clause G1 or clause G2, wherein at least one of the main constriction region, if present, and the bypass constriction region includes a section of uniform cross sectional area, the section having a cross sectional area smaller than that of the respective air inlet of the flow channel.

G4. The smoking substitute apparatus according to any one of clauses G1 to G3, wherein at least one of the main constriction region, if present, and the bypass constriction region includes a mesh which partially blocks the flow channel in which it is disposed.

G5. The smoking substitute apparatus according to any one of clauses G1 to G4, wherein at least one of the main constriction region, if present, and the bypass constriction region is configured to generate turbulent air flow along the flow channel in which it is disposed.

G6. The smoking substitute apparatus according to clause G5, wherein the at least one of the main constriction region, if present, and the bypass constriction region includes one or more walls having a surface that is rougher than that of the walls of the remainder of the flow channel in which it is disposed.

G7. The smoking substitute apparatus according to any one of clauses G1 to G6, wherein at least one of the main constriction region, if present, and the bypass constriction region is adjustable to change the resistance to flow along the flow channel in which it is disposed.

G8. The smoking substitute apparatus according to clause G7, wherein the at least one of the main constriction region, if present, and the bypass constriction region includes a valve.

G9. The smoking substitute apparatus according to any one of clauses G7 and G8, wherein the at least one of the main constriction region, if present, and the bypass constriction region includes two relatively rotatable members, rotatable between a first relative position and a second relative position, these positions differing in terms of the cumulative obstruction presented to air flow.

G10. The smoking substitute apparatus according to any one of clauses G1 to G9, wherein the smoking substitute apparatus is configured to generate an aerosol having a median droplet size, d50, of at least 1 μm.

G11. The smoking substitute apparatus according to any one of clauses G1 to G10, wherein the aerosol generator includes a heater.

G12. The smoking substitute apparatus according to any one of the previous clauses G1 to G11, wherein the aerosol precursor is a liquid.

G13. A smoking substitute device configured to engage with the smoking substitute apparatus according to any one of clauses G1 to G12; wherein the device comprises a controller and a power source configured to energize the aerosol generator.

G14. A smoking substitute system for generating an aerosol, comprising: the smoking substitute apparatus according to any one of the previous clauses G1 to G12; and the smoking substitute device of clause G13.

G15. A method of generating aerosol using the smoking substitute apparatus of any one of clauses G1 to G12, wherein droplets of the aerosol have a median diameter, d50, of at least 1 μm.

Development H

H1. A smoking substitute apparatus (150a, 150b, 150c, 150d) comprising:
one or more air inlets (172, 176, 182);
one or more outlets (174, 184) arranged at a mouthpiece (154);
a first passage (170) leading from at least one of the air inlets (172, 176) to at least one of the outlets (174) at the mouthpiece (154);
an aerosol generator arranged in a vaporization chamber in the first passage (170), the aerosol generator being operable to generate an aerosol from an aerosol precursor (160), to flow in use along the first passage (170) downstream of the aerosol generator for inhalation by a user drawing on the mouthpiece (154); and
a second passage (180) leading from at least one of the air inlets (176, 182) to at least one of the outlets (174, 184) at the mouthpiece (154) wherein the second passage (180) bypasses the vaporization chamber of the first passage (170);
wherein the mouthpiece (154) comprises a flow constrictor (190a, 190b, 190c) for constricting the flow of air in the second passage (180).

H2. A smoking substitute apparatus (150b) according to clause H1, wherein the mouthpiece (154b) is a releasably engageable part of the smoking substitute apparatus (150).

H3. A smoking substitute apparatus (150c) according to either of clauses H1 or H2, wherein: the flow constrictor (190c) is a releasably engageable component of the mouthpiece (154c).

H4. A smoking substitute apparatus (150a, 150b, 150c) according to any preceding clause H1 to H3, wherein: the aerosol generator comprises a heater operable to generate the aerosol from the aerosol precursor (160).

H5. A smoking substitute apparatus (150a, 150b, 150c) according to any preceding clause H1 to H4, wherein: the aerosol generator comprises a porous wick which, in use, wicks aerosol precursor (160) from a reservoir (152) to the first passage (170) for entrainment in air flowing downstream of the aerosol generator.

H6. A smoking substitute apparatus (150a, 150b, 150c) according to clause H5 as dependent on claim 4, wherein: the heater comprises a heating filament (164) that is wound around a portion of the porous wick (162).

H7. A smoking substitute apparatus (150a, 150b, 150c) according to any preceding clause H1 to H6, wherein: the first passage (170), the second passage (180) and flow constrictor (190a, 190b, 190c) are configured such that, in use, the flow rate in the first passage (170) is more than 1/20 of the flow rate in the second passage (180).

H8. A smoking substitute apparatus (150a, 150b, 150c) according to clause H7, wherein: the first passage (170), the second passage (180) and flow constrictor (190a, 190b, 190c) are configured such that, in use, the flow rate in the first passage (170) is less than twice of the flow rate in the second passage (180).

H9. A smoking substitute apparatus (150a) according to any preceding clause H1 to H8, wherein: in use, the d50 particle size of the aerosol particles generated by the aerosol generator is greater than 1 μm and less than 10 μm

H10. A smoking substitute apparatus (150a) according to any preceding clause H1 to H9, wherein: in use, the span of particle size distribution, defined as (d90−d10)/d50, is less than 20.

H11. A smoking substitute apparatus (150a, 150b, 150c) according to any one of clause H1 to clause H10, wherein the smoking substitute apparatus is comprised by or within a cartridge configured for engagement with a base unit, the cartridge and base unit together forming a smoking substitute system.

H12. A smoking substitute system comprising:
a base unit (120), and
a smoking substitute apparatus (150a, 150b, 150c) according to clause H11, wherein the smoking substitute apparatus is removably engageable with the base unit.

H13. A kit of parts for a smoking substitute apparatus, comprising
a smoking substitute apparatus (150b) according to clause H2; and
a plurality of mouthpieces (154b) configured for engagement with the smoking substitute apparatus (150b) according to clause H2; wherein
each of the plurality of mouthpieces comprises a flow constrictor (190b) which is configured to constrict the flow of air in the second passage (180) by a respectively different level of constriction.

H14. A kit of parts for a smoking substitute apparatus, comprising
a smoking substitute apparatus (150c) according to clause H3; and
a plurality of flow constrictors (190c) configured for engagement with the mouthpiece (154c) of the smoking substitute apparatus (150c) according to clause H3; wherein
each of the plurality of flow constrictors (190c) is configured to constrict the flow of air in the second passage (180) by a respectively different level of constriction.

H15. A method of using a smoking substitute apparatus according to any one of clauses H1 to H11 to generate an aerosol, comprising the step of selecting a flow constrictor providing a desired level of constriction from a plurality of flow constrictors providing different levels of constriction.

Development I

I1. A smoking substitute apparatus comprising:
a vaporizer chamber, including a vaporizer configured to vaporize a vaporizable liquid;
a primary airflow path, which passes from a first air inlet of the smoking substitute apparatus through the vaporizer chamber, to an outlet of the smoking substitute apparatus;
a secondary airflow path, which passes from a second air inlet of the smoking substitute apparatus to the outlet of the smoking substitute apparatus, said secondary airflow path bypassing the vaporizer chamber and passing through one or more bypass air ducts;
wherein one or more adjustable airflow restrictors are disposed along either or both of the primary airflow path and the secondary airflow path, said adjustable airflow restrictors being adjustable by a user of the device to vary a draw resistance of the smoking substitute apparatus.

I2. The smoking substitute apparatus of clause I1, wherein the or each airflow restrictor is a constriction along the respective airflow path, the constriction having an adjustable cross-section.

I3. The smoking substitute apparatus of either clause I1 or clause I2, wherein the or each airflow restrictor is adjustable through the application of a force, by the user, to a portion of an outer housing of the smoking substitute apparatus.

I4. The smoking substitute apparatus of either clause I1 or clause I2, wherein the or each airflow restrictor is adjustable through adjustment of a dial attached to a portion of an outer housing of the smoking substitute apparatus.

I5. The smoking substitute apparatus of any preceding clause I1 to I4, wherein the or each airflow restrictor disposed along the secondary airflow path is located within the or each bypass air duct.

I6. The smoking substitute apparatus of any preceding clause I1 to I5, wherein the or each airflow restrictor disposed along the primary airflow path is located between the first air inlet and a vaporizer chamber inlet of the vaporizer chamber.

I7. The smoking substitute apparatus of any preceding clause I1 to I6, including two first air inlets, located on respectively opposite sides of the smoking substitute apparatus.

I8. The smoking substitute apparatus of any preceding clause I1 to I7, including two second air inlets, located on respectively opposite sides of the smoking substitute apparatus.

I9. The smoking substitute apparatus of any preceding clause I1 to I8, wherein the first air inlet provides air to both the primary and secondary airflow path.

I10. The smoking substitute apparatus of any preceding clause I1 to I9, wherein the primary airflow path and secondary airflow path converge in the outlet of the smoking substitute apparatus.

I11. The smoking substitute apparatus of any preceding clause I1 to I10, wherein the primary airflow path and the secondary airflow path partially overlap, and wherein the or each airflow restrictor is disposed along the overlapping portion of the airflow paths.

I12. The smoking substitute apparatus of any preceding clause I1 to I11, the vaporizer chamber including a flow straightener located between a vaporizer chamber inlet and the vaporizer, configured to induce a laminar airflow over the vaporizer.

I13. The smoking substitute apparatus of any preceding clause I1 to I12, the vaporizer chamber including a plenum, located between a vaporizer chamber inlet and the vaporizer, configured to reduce an airflow velocity over the vaporizer.

I14. The smoking substitute apparatus of any preceding clause I1 to I13, including a vaporizer chamber outlet, located between the vaporizer and the outlet of the smoking substitute apparatus, wherein the vaporizer chamber outlet is a tapered chimney.

I15. A smoking substitute system, including the smoking substitute apparatus of any preceding clause I1 to I14 and a main body, the main body including a power source for the vaporizer.

What is claimed is:

1. A smoking substitute apparatus comprising:
an air inlet;
a first passage leading from the air inlet to a first outlet
an aerosol generator arranged in fluid communication with the first passage, the aerosol generator being operable to generate an aerosol from an aerosol precursor, to flow in use along the first passage downstream of the aerosol generator for inhalation by a user drawing on the first outlet,
the apparatus further comprises a second passage leading from the air inlet to a second outlet,
characterized in that
the second outlet is separate from the first outlet, wherein, downstream of the aerosol generator, the second passage and the second outlet bypass the first passage and the first outlet.

2. A smoking substitute apparatus according to claim 1, wherein:
the first passage comprises a vaporization chamber in which the aerosol generator is arranged, the vaporization chamber being bypassed by the second passage, and wherein
the vaporization chamber has a larger cross sectional diameter than a downstream part of the first passage.

3. A smoking substitute apparatus according to either of claims 1 and 2, wherein:
the aerosol generator comprises a heater operable to generate the aerosol from the aerosol precursor.

4. A smoking substitute apparatus according to any preceding claim, wherein:
the aerosol generator comprises a porous wick which, in use, wicks aerosol precursor from a reservoir to the first passage for entrainment in air flowing downstream of the aerosol generator.

5. A smoking substitute apparatus according to claim 4 as dependent on claim 3, wherein:
the heater comprises a heating filament that is wound around a portion of the porous wick.

6. A smoking substitute apparatus according to any preceding claim, wherein:
the part of the first passage bypassed by the second passage comprises a flow conditioning apparatus arranged upstream of the aerosol generator, wherein the flow conditioning apparatus is configured to reduce the turbulence in flow at the aerosol generator.

7. A smoking substitute apparatus according to claim 6, wherein:
the flow conditioning apparatus comprises a mesh arranged in the first passage such that, in use, the flow generated by a user drawing on the first outlet passes through the mesh.

8. A smoking substitute apparatus according to claim 6, wherein:
the flow conditioning apparatus comprises a mesh arranged in the first passage, wherein the mesh comprises an air-permeable material or comprises an air-impermeable material with one or more bores extending through.

9. A smoking substitute apparatus according to any preceding claim, wherein:
the first passage and the second passage are configured such that, in use, the flow rate in the first passage is more than 1/20 of the flow rate in the second passage.

10. A smoking substitute apparatus according to claim 9, wherein:
the first passage and the second passage are configured such that, in use, the flow rate in the first passage is less than twice of the flow rate in the second passage.

11. A smoking substitute apparatus according to any preceding claim, wherein:
in use, the d50 particle size of the aerosol particles generated by the aerosol generator is greater than 1 μm.

12. A smoking substitute apparatus according to any preceding claim, wherein:
in use, the d50 particle size of the aerosol particles generated by the aerosol generator is less than 10 μm.

13. A smoking substitute apparatus according to any one of claim 1 to claim 12, wherein the smoking substitute apparatus is comprised by or within a cartridge configured for engagement with a base unit, the cartridge and base unit together forming a smoking substitute system.

14. A smoking substitute system comprising:
a base unit, and
a smoking substitute apparatus according to claim 13, wherein the smoking substitute apparatus is removably engageable with the base unit.

15. A method of using a smoking substitute apparatus according to any one of claims 1 to 12 to generate an aerosol.
